SEQUENCE LISTING (399 sequences)

SEQ ID NO: 1 - DNA, 26 bp, synthetic construct
  source 1..26: mol_type = other DNA
gaatgccctgacagctctcgcttata

SEQ ID NO: 2 - DNA, 41 bp, synthetic construct
  source 1..41: mol_type = other DNA
ctcagagaagtctgaaatattcgatgatcaattctcagttg

SEQ ID NO: 3 - DNA, 34 bp, synthetic construct
  source 1..34: mol_type = other DNA
ccaaatcgmttctcacctaaatctccagacaaag

SEQ ID NO: 4 - DNA, 26 bp, synthetic construct
  source 1..26: mol_type = other DNA
cacctgactctccagacaaagctcat

SEQ ID NO: 5 - DNA, 22 bp, synthetic construct
  source 1..22: mol_type = other DNA
cctgaatgccccaacagctctc

SEQ ID NO: 6 - DNA, 24 bp, synthetic construct
  source 1..24: mol_type = other DNA
gattctcagggcgccagttctcta

SEQ ID NO: 7 - DNA, 31 bp, synthetic construct
  source 1..31: mol_type = other DNA
cctaattgattctcagctcaccacgtccata

SEQ ID NO: 8 - DNA, 19 bp, synthetic construct
  source 1..19: mol_type = other DNA
tcagggcgccagttccatg

SEQ ID NO: 9 - DNA, 29 bp, synthetic construct
  source 1..29: mol_type = other DNA
tcctagattctcaggtctccagttcccta

SEQ ID NO: 10 - DNA, 28 bp, synthetic construct
  source 1..28: mol_type = other DNA
gaggaaacttccctgatcgattctcagc

SEQ ID NO: 11 - DNA, 27 bp, synthetic construct
  source 1..27: mol_type = other DNA
caacttccctgatcgattctcaggtca

SEQ ID NO: 12 - DNA, 30 bp, synthetic construct
  source 1..30: mol_type = other DNA
aggaaacttccctgatcaattctcaggtca

SEQ ID NO: 13 - DNA, 29 bp, synthetic construct
  source 1..29: mol_type = other DNA
ggaaacttccctcctagattttcaggtcg

SEQ ID NO: 14 - DNA, 27 bp, synthetic construct
  source 1..27: mol_type = other DNA
ccccaatggctacaatgtctccagatt

SEQ ID NO: 15 - DNA, 23 bp, synthetic construct
  source 1..23: mol_type = other DNA
ggagaggtccctgatggctacaa

SEQ ID NO: 16 - DNA, 28 bp, synthetic construct
  source 1..28: mol_type = other DNA
tccctgatggttatagtgtctccagagc

SEQ ID NO: 17 - DNA, 27 bp, synthetic construct
  source 1..27: mol_type = other DNA
ggagaagtccccaatggctacaatgtc

SEQ ID NO: 18 - DNA, 26 bp, synthetic construct
  source 1..26: mol_type = other DNA
aaaggagaagtcccgaatggctacaa

SEQ ID NO: 19 - DNA, 29 bp, synthetic construct
  source 1..29: mol_type = other DNA
gttcccaatggctacaatgtctccagatc

SEQ ID NO: 20 - DNA, 32 bp, synthetic construct
  source 1..32: mol_type = other DNA
gaagtccccaatggctacaatgtctctagatt

SEQ ID NO: 21 - DNA, 26 bp, synthetic construct
  source 1..26: mol_type = other DNA
gagaagtccccgatggctacaatgta

SEQ ID NO: 22 - DNA, 24 bp, synthetic construct
  source 1..24: mol_type = other DNA
gtgatcggttctctgcacagaggt

SEQ ID NO: 23 - DNA, 23 bp, synthetic construct
  source 1..23: mol_type = other DNA
cgcttctctgcagagaggactgg

SEQ ID NO: 24 - DNA, 22 bp, synthetic construct
  source 1..22: mol_type = other DNA
ggttctttgcagtcaggcctga

SEQ ID NO: 25 - DNA, 22 bp, synthetic construct
  source 1..22: mol_type = other DNA
cagtggtcggttctctgcagag

SEQ ID NO: 26 - DNA, 29 bp, synthetic construct
  source 1..29: mol_type = other DNA
gctcagtgatcaattctccacagagaggt

SEQ ID NO: 27 - DNA, 22 bp, synthetic construct
  source 1..22: mol_type = other DNA
ttctctgcagagaggcctgagg

SEQ ID NO: 28 - DNA, 25 bp, synthetic construct
  source 1..25: mol_type = other DNA
cccagtgatcgcttctttgcagaaa

SEQ ID NO: 29 - DNA, 23 bp, synthetic construct
  source 1..23: mol_type = other DNA
ctgcagagaggcctaagggatct

SEQ ID NO: 30 - DNA, 33 bp, synthetic construct
  source 1..33: mol_type = other DNA
gaagggtacaatgtctctggaaacaaactcaag

SEQ ID NO: 31 - DNA, 31 bp, synthetic construct
  source 1..31: mol_type = other DNA
ggggtactgtgtttcttgaaacaagcttgag

SEQ ID NO: 32 - DNA, 30 bp, synthetic construct
  source 1..30: mol_type = other DNA
cagttccctgacttgcactctgaactaaac

SEQ ID NO: 33 - DNA, 32 bp, synthetic construct
  source 1..32: mol_type = other DNA
actaacaaaggagaagtctcagatggctacag

SEQ ID NO: 34 - DNA, 27 bp, synthetic construct
  source 1..27: mol_type = other DNA
agataaaggagaagtccccgatggcta

SEQ ID NO: 35 - DNA, 35 bp, synthetic construct
  source 1..35: mol_type = other DNA
gatactgacaaaggagaagtctcagatggctatag

SEQ ID NO: 36 - DNA, 29 bp, synthetic construct
  source 1..29: mol_type = other DNA
ctaaggatcgattttctgcagagaggctc

SEQ ID NO: 37 - DNA, 27 bp, synthetic construct
  source 1..27: mol_type = other DNA
ttgattctcagcacagatgcctgatgt

SEQ ID NO: 38 - DNA, 24 bp, synthetic construct
  source 1..24: mol_type = other DNA
attctcagctgagaggcctgatgg

SEQ ID NO: 39 - DNA, 30 bp, synthetic construct
  source 1..30: mol_type = other DNA
ggatcgattctcagctaagatgcctaatgc

SEQ ID NO: 40 - DNA, 28 bp, synthetic construct
  source 1..28: mol_type = other DNA
ctcagcagagatgcctgatgcaacttta

SEQ ID NO: 41 - DNA, 29 bp, synthetic construct
  source 1..29: mol_type = other DNA
ctgatcgattctcagctcaacagttcagt

SEQ ID NO: 42 - DNA, 26 bp, synthetic construct
  source 1..26: mol_type = other DNA
tagctgaaaggactggagggacgtat

SEQ ID NO: 43 - DNA, 24 bp, synthetic construct
  source 1..24: mol_type = other DNA
ccaggaggccgaacacttctttct

SEQ ID NO: 44 - DNA, 25 bp, synthetic construct
  source 1..25: mol_type = other DNA
gctaagtgcctcccaaattcaccct

SEQ ID NO: 45 - DNA, 26 bp, synthetic construct
  source 1..26: mol_type = other DNA
cacagctgaaagacctaacggaacgt

SEQ ID NO: 46 - DNA, 24 bp, synthetic construct
  source 1..24: mol_type = other DNA
ctgctgaatttcccaaagagggcc

SEQ ID NO: 47 - DNA, 20 bp, synthetic construct
  source 1..20: mol_type = other DNA
agggtacagcgtctctcggg

SEQ ID NO: 48 - DNA, 23 bp, synthetic construct
  source 1..23: mol_type = other DNA
gcctgaccttgtccactctgaca

SEQ ID NO: 49 - DNA, 27 bp, synthetic construct
  source 1..27: mol_type = other DNA
atgagcgatttttagcccaatgctcca

SEQ ID NO: 50 - DNA, 24 bp, synthetic construct
  source 1..24: mol_type = other DNA
tgaaggctacgtgtctgccaagag

SEQ ID NO: 51 - DNA, 24 bp, synthetic construct
  source 1..24: mol_type = other DNA
ctcatctcaatgccccaagaacgc

SEQ ID NO: 52 - DNA, 31 bp, synthetic construct
  source 1..31: mol_type = other DNA
agatctctgatggatacagtgtctctcgaca

SEQ ID NO: 53 - DNA, 34 bp, synthetic construct
  source 1..34: mol_type = other DNA
agatctttcctctgagtcaacagtctccagaata

SEQ ID NO: 54 - DNA, 35 bp, synthetic construct
  source 1..35: mol_type = other DNA
cactgaaaaaggagatatctctgaggggtatcatg

SEQ ID NO: 55 - DNA, 31 bp, synthetic construct
  source 1..31: mol_type = other DNA
gttcctgaagggtacaaagtctctcgaaaag

SEQ ID NO: 56 - DNA, 27 bp, synthetic construct
  source 1..27: mol_type = other DNA
ctgaggggtacagtgtctctagagaga

SEQ ID NO: 57 - DNA, 25 bp, synthetic construct
  source 1..25: mol_type = other DNA
agccgcccaaacctaacattctcaa

SEQ ID NO: 58 - DNA, 18 bp, synthetic construct
  source 1..18: mol_type = other DNA
cccaggaccggcagttca

SEQ ID NO: 59 - DNA, 40 bp, synthetic construct
  source 1..40: mol_type = other DNA
ttgattagagacatatccctattgaaaatatttcctggca

SEQ ID NO: 60 - DNA, 31 bp, synthetic construct
  source 1..31: mol_type = other DNA
agatgccctgagtcagcatagtcattctaac

SEQ ID NO: 61 - DNA, 26 bp, synthetic construct
  source 1..26: mol_type = other DNA
ggaggggaaggccccacagcgtcttc

SEQ ID NO: 62 - DNA, 27 bp, synthetic construct
  source 1..27: mol_type = other DNA
tgaagtcatacagttcctggtgtccat

SEQ ID NO: 63 - DNA, 28 bp, synthetic construct
  source 1..28: mol_type = other DNA
ccaaatcaggctttggagcacctgatct

SEQ ID NO: 64 - DNA, 33 bp, synthetic construct
  source 1..33: mol_type = other DNA
ccaaacaaaggcttagaatatttattacatgtc

SEQ ID NO: 65 - DNA, 27 bp, synthetic construct
  source 1..27: mol_type = other DNA
ccaggtccctgaggcactccaccagct

SEQ ID NO: 66 - DNA, 28 bp, synthetic construct
  source 1..28: mol_type = other DNA
ctgaatctaaattatgagccatctgaca

SEQ ID NO: 67 - DNA, 32 bp, synthetic construct
  source 1..32: mol_type = other DNA
tcattccttagtcgctctgatagttatggtta

SEQ ID NO: 68 - DNA, 31 bp, synthetic construct
  source 1..31: mol_type = other DNA
cattccttagtcggtctaaagggtacagtta

SEQ ID NO: 69 - DNA, 31 bp, synthetic construct
  source 1..31: mol_type = other DNA
acaacatgacctatgaacggttctcttcatc

SEQ ID NO: 70 - DNA, 31 bp, synthetic construct
  source 1..31: mol_type = other DNA
ctgaatttaacaagagccaaacctccttcca

SEQ ID NO: 71 - DNA, 25 bp, synthetic construct
  source 1..25: mol_type = other DNA
ccgacagaaagtccagcactctgag

SEQ ID NO: 72 - DNA, 36 bp, synthetic construct
  source 1..36: mol_type = other DNA
cactgttctattgaataaaaaggataaacatctgtc

SEQ ID NO: 73 - DNA, 32 bp, synthetic construct
  source 1..32: mol_type = other DNA
gtcacctttgataccacccttaaacagagttt

SEQ ID NO: 74 - DNA, 35 bp, synthetic construct
  source 1..35: mol_type = other DNA
agactaaatgctacattactgaagaatggaagcag

SEQ ID NO: 75 - DNA, 36 bp, synthetic construct
  source 1..36: mol_type = other DNA
tgaggctgaatttataaagagtaaattctcctttaa

SEQ ID NO: 76 - DNA, 32 bp, synthetic construct
  source 1..32: mol_type = other DNA
gctgaatttaagaagagtgaaacctccttcca

SEQ ID NO: 77 - DNA, 33 bp, synthetic construct
  source 1..33: mol_type = other DNA
ggctgaatttaagaggagtcaatcttccttcaa

SEQ ID NO: 78 - DNA, 30 bp, synthetic construct
  source 1..30: mol_type = other DNA
gacacttatcacttccccaatcaatacccc

SEQ ID NO: 79 - DNA, 33 bp, synthetic construct
  source 1..33: mol_type = other DNA
ggctgaatttaacaagagtcaaacttccttcca

SEQ ID NO: 80 - DNA, 32 bp, synthetic construct
  source 1..32: mol_type = other DNA
gctgaatttaagaagagcgaaacctccttcta

SEQ ID NO: 81 - DNA, 31 bp, synthetic construct
  source 1..31: mol_type = other DNA
ccatgtaccgtaaagaaaccacttctttcca

SEQ ID NO: 82 - DNA, 31 bp, synthetic construct
  source 1..31: mol_type = other DNA
ccacataccgtaaagaaaccacttctttcca

SEQ ID NO: 83 - DNA, 25 bp, synthetic construct
  source 1..25: mol_type = other DNA
tggatgcagacacaaagcaaagctc

SEQ ID NO: 84 - DNA, 36 bp, synthetic construct
  source 1..36: mol_type = other DNA
taaagaactgcttggaaaagaaaaattttatagtgt

SEQ ID NO: 85 - DNA, 30 bp, synthetic construct
  source 1..30: mol_type = other DNA
acagctcaatagagccagccagtatatttc

SEQ ID NO: 86 - DNA, 29 bp, synthetic construct
  source 1..29: mol_type = other DNA
cagctcaataaagccagccagtatgtttc

SEQ ID NO: 87 - DNA, 32 bp, synthetic construct
  source 1..32: mol_type = other DNA
gcacaggtcgataaatccagcaagtatatctc

SEQ ID NO: 88 - DNA, 34 bp, synthetic construct
  source 1..34: mol_type = other DNA
ctgttacattgaacaagacagccaaacatttctc

SEQ ID NO: 89 - DNA, 36 bp, synthetic construct
  source 1..36: mol_type = other DNA
caccgttttattgaataagacagtgaaacatctctc

SEQ ID NO: 90 - DNA, 24 bp, synthetic construct
  source 1..24: mol_type = other DNA
ccagaaggcaagaaaatccgccaa

SEQ ID NO: 91 - DNA, 33 bp, synthetic construct
  source 1..33: mol_type = other DNA
agaagcgcttggaaaagagaagttttatagtgt

SEQ ID NO: 92 - DNA, 30 bp, synthetic construct
  source 1..30: mol_type = other DNA
tgaccttaacaaaggcgagacatctttcca

SEQ ID NO: 93 - DNA, 28 bp, synthetic construct
  source 1..28: mol_type = other DNA
cgcttgacacttccaagaaaagcagttc

SEQ ID NO: 94 - DNA, 30 bp, synthetic construct
  source 1..30: mol_type = other DNA
cagtcctatcaagagtgacagttccttcca

SEQ ID NO: 95 - DNA, 30 bp, synthetic construct
  source 1..30: mol_type = other DNA
gaacttccagaaatccaccagttccttcaa

SEQ ID NO: 96 - DNA, 33 bp, synthetic construct
  source 1..33: mol_type = other DNA
gctaaaagccacattaacaaagaaggaaagctt

SEQ ID NO: 97 - DNA, 30 bp, synthetic construct
  source 1..30: mol_type = other DNA
ctcgctggataaatcatcaggacgtagtac

SEQ ID NO: 98 - DNA, 22 bp, synthetic construct
  source 1..22: mol_type = other DNA
tcgctacggaacgctacagctt

SEQ ID NO: 99 - DNA, 30 bp, synthetic construct
  source 1..30: mol_type = other DNA
ctccttcaataaaagtgccaagcagttctc

SEQ ID NO: 100 - DNA, 30 bp, synthetic construct
  source 1..30: mol_type = other DNA
cactcttaataccaaggagggttacagcta

SEQ ID NO: 101 - DNA, 30 bp, synthetic construct
  source 1..30: mol_type = other DNA
tcagtttggagaagcaaaaaagaacagctc

SEQ ID NO: 102 - DNA, 30 bp, synthetic construct
  source 1..30: mol_type = other DNA
gatcatcacagaagacagaaagtccagcac

SEQ ID NO: 103 - DNA, 29 bp, synthetic construct
  source 1..29: mol_type = other DNA
gcaatcgctgaagacagaaagtccagtac

SEQ ID NO: 104 - DNA, 29 bp, synthetic construct
  source 1..29: mol_type = other DNA
cagtttggtgatgcaagaaaggacagttc

SEQ ID NO: 105 - DNA, 28 bp, synthetic construct
  source 1..28: mol_type = other DNA
cagtcaaagctgaggaactttatggcca

SEQ ID NO: 106 - DNA, 30 bp, synthetic construct
  source 1..30: mol_type = other DNA
cttcttaaacaaaagtgccaagcacctctc

SEQ ID NO: 107 - DNA, 34 bp, synthetic construct
  source 1..34: mol_type = other DNA
ctgcttcatttaatgaaaaaaagcagcaaagctc

SEQ ID NO: 108 - DNA, 36 bp, synthetic construct
  source 1..36: mol_type = other DNA
ttctgtgagcttccagaaaacaactaaaactattca

SEQ ID NO: 109 - DNA, 36 bp, synthetic construct
  source 1..36: mol_type = other DNA
cactgtactgttgaataaaaatgctaaacatgtctc

SEQ ID NO: 110 - DNA, 36 bp, synthetic construct
  source 1..36: mol_type = other DNA
gcctgtgaactttgaaaaaaagaaaaagttcatcaa

SEQ ID NO: 111 - DNA, 31 bp, synthetic construct
  source 1..31: mol_type = other DNA
ccaagttggatgagaaaaagcagcaaagttc

SEQ ID NO: 112 - DNA, 30 bp, synthetic construct
  source 1..30: mol_type = other DNA
tcagtttggtataaccagaaaggacagctt

SEQ ID NO: 113 - DNA, 36 bp, synthetic construct
  source 1..36: mol_type = other DNA
aagtagcatattagataagaaagaactttccagcat

SEQ ID NO: 114 - DNA, 30 bp, synthetic construct
  source 1..30: mol_type = other DNA
caggcttaaaaaaggagaccagcacatttc

SEQ ID NO: 115 - DNA, 27 bp, synthetic construct
  source 1..27: mol_type = other DNA
cttccagaaagcagccaaatccttcag

SEQ ID NO: 116 - DNA, 24 bp, synthetic construct
  source 1..24: mol_type = other DNA
tgataccaaagcccgtctcagcac

SEQ ID NO: 117 - DNA, 31 bp, synthetic construct
  source 1..31: mol_type = other DNA
gaggcggaaatattaaagacaaaaactcccc

SEQ ID NO: 118 - DNA, 31 bp, synthetic construct
  source 1..31: mol_type = other DNA
ccacaataaacatacaggaaaagcacagctc

SEQ ID NO: 119 - DNA, 22 bp, synthetic construct
  source 1..22: mol_type = other DNA
agaaagcagcgaaatccgtcgc

SEQ ID NO: 120 - DNA, 30 bp, synthetic construct
  source 1..30: mol_type = other DNA
tgacattgatattgcaaagaacctggctgt

SEQ ID NO: 121 - DNA, 30 bp, synthetic construct
  source 1..30: mol_type = other DNA
gaaacacattctgacccagaaagcctttca

SEQ ID NO: 122 - DNA, 83 bp, synthetic construct
  source 1..83: mol_type = other DNA
aatgatacggcgaccaccgagatctacactctttccctacacgacgctcttccgatctggggagaagtggaaactctggttcc

SEQ ID NO: 123 - DNA, 90 bp, synthetic construct
  source 1..90: mol_type = other DNA
aatgatacggcgaccaccgagatctacactctttccctacacgacgctcttccgatctgactcaccagatataatgaatacatgggtccc

SEQ ID NO: 124 - DNA, 79 bp, synthetic construct
  source 1..79: mol_type = other DNA
aatgatacggcgaccaccgagatctacactctttccctacacgacgctcttccgatctccggatgctgagtctggtccc

SEQ ID NO: 125 - DNA, 78 bp, synthetic construct
  source 1..78: mol_type = other DNA
aatgatacggcgaccaccgagatctacactctttccctacacgacgctcttccgatcttgtacagccagcctggtccc

SEQ ID NO: 126 - DNA, 84 bp, synthetic construct
  source 1..84: mol_type = other DNA
aatgatacggcgaccaccgagatctacactctttccctacacgacgctcttccgatctcttggttgcacttggagtcttgttcc

SEQ ID NO: 127 - DNA, 84 bp, synthetic construct
  source 1..84: mol_type = other DNA
aatgatacggcgaccaccgagatctacactctttccctacacgacgctcttccgatctcacggatgaacaataaggctggttcc

SEQ ID NO: 128 - DNA, 83 bp, synthetic construct
  source 1..83: mol_type = other DNA
aatgatacggcgaccaccgagatctacactctttccctacacgacgctcttccgatctttggtatgaccaccacttggttccc

SEQ ID NO: 129 - DNA, 79 bp, synthetic construct
  source 1..79: mol_type = other DNA
aatgatacggcgaccaccgagatctacactctttccctacacgacgctcttccgatctactgaccagaagtcgggtgcc

SEQ ID NO: 130 - DNA, 94 bp, synthetic construct
  source 1..94: mol_type = other DNA
aatgatacggcgaccaccgagatctacactctttccctacacgacgctcttccgatctatggaacttacttgctttaacaaatagtcttgttcc

SEQ ID NO: 131 - DNA, 85 bp, synthetic construct
  source 1..85: mol_type = other DNA
aatgatacggcgaccaccgagatctacactctttccctacacgacgctcttccgatctactgagttccacttttagctgagtgcc

SEQ ID NO: 132 - DNA, 85 bp, synthetic construct
  source 1..85: mol_type = other DNA
aatgatacggcgaccaccgagatctacactctttccctacacgacgctcttccgatctacctggagagactagaagcatagtccc

SEQ ID NO: 133 - DNA, 79 bp, synthetic construct
  source 1..79: mol_type = other DNA
aatgatacggcgaccaccgagatctacactctttccctacacgacgctcttccgatctcctgaccagcagtctggtccc

SEQ ID NO: 134 - DNA, 84 bp, synthetic construct
  source 1..84: mol_type = other DNA
aatgatacggcgaccaccgagatctacactctttccctacacgacgctcttccgatctcttgggatgacttggagctttgttcc

SEQ ID NO: 135 - DNA, 92 bp, synthetic construct
  source 1..92: mol_type = other DNA
aatgatacggcgaccaccgagatctacactctttccctacacgacgctcttccgatctcctacttaccaggttttactgataatcttgtccc

SEQ ID NO: 136 - DNA, 86 bp, synthetic construct
  source 1..86: mol_type = other DNA
aatgatacggcgaccaccgagatctacactctttccctacacgacgctcttccgatcttactggaactcactgataaggtggttcc

SEQ ID NO: 137 - DNA, 85 bp, synthetic construct
  source 1..85: mol_type = other DNA
aatgatacggcgaccaccgagatctacactctttccctacacgacgctcttccgatctctggaactcactgataaggtgggttcc

SEQ ID NO: 138 - DNA, 90 bp, synthetic construct
  source 1..90: mol_type = other DNA
aatgatacggcgaccaccgagatctacactctttccctacacgacgctcttccgatcttacttactaagatccacctttaacatggttcc

SEQ ID NO: 139 - DNA, 85 bp, synthetic construct
  source 1..85: mol_type = other DNA
aatgatacggcgaccaccgagatctacactctttccctacacgacgctcttccgatctacttggtttaactagcaccctggttcc

SEQ ID NO: 140 - DNA, 82 bp, synthetic construct
  source 1..82: mol_type = other DNA
aatgatacggcgaccaccgagatctacactctttccctacacgacgctcttccgatctaggccagacagtcaactgagttcc

SEQ ID NO: 141 - DNA, 92 bp, synthetic construct
  source 1..92: mol_type = other DNA
aatgatacggcgaccaccgagatctacactctttccctacacgacgctcttccgatctctcacttacttggagtgacattatgtttggatcc

SEQ ID NO: 142 - DNA, 90 bp, synthetic construct
  source 1..90: mol_type = other DNA
aatgatacggcgaccaccgagatctacactctttccctacacgacgctcttccgatcttacttacttgctcttacagttactgtggttcc

SEQ ID NO: 143 - DNA, 91 bp, synthetic construct
  source 1..91: mol_type = other DNA
aatgatacggcgaccaccgagatctacactctttccctacacgacgctcttccgatcttaacttacttggttttacattgagtttggtccc

SEQ ID NO: 144 - DNA, 86 bp, synthetic construct
  source 1..86: mol_type = other DNA
aatgatacggcgaccaccgagatctacactctttccctacacgacgctcttccgatcttaccaggtaaaacagtcaattgtgtccc

SEQ ID NO: 145 - DNA, 85 bp, synthetic construct
  source 1..85: mol_type = other DNA
aatgatacggcgaccaccgagatctacactctttccctacacgacgctcttccgatctactgggtttcacagataactccgttcc

SEQ ID NO: 146 - DNA, 79 bp, synthetic construct
  source 1..79: mol_type = other DNA
aatgatacggcgaccaccgagatctacactctttccctacacgacgctcttccgatctggtgaccacaacctgggtccc

SEQ ID NO: 147 - DNA, 80 bp, synthetic construct
  source 1..80: mol_type = other DNA
aatgatacggcgaccaccgagatctacactctttccctacacgacgctcttccgatctgcttgacaagcagccttgtccc

SEQ ID NO: 148 - DNA, 80 bp, synthetic construct
  source 1..80: mol_type = other DNA
aatgatacggcgaccaccgagatctacactctttccctacacgacgctcttccgatctgcagcacggacaatctggttcc

SEQ ID NO: 149 - DNA, 81 bp, synthetic construct
  source 1..81: mol_type = other DNA
aatgatacggcgaccaccgagatctacactctttccctacacgacgctcttccgatctggcttcacagtgagcgtagtccc

SEQ ID NO: 150 - DNA, 84 bp, synthetic construct
  source 1..84: mol_type = other DNA
aatgatacggcgaccaccgagatctacactctttccctacacgacgctcttccgatctcttggtatgaccgagagtttggtccc

SEQ ID NO: 151 - DNA, 85 bp, synthetic construct
  source 1..85: mol_type = other DNA
aatgatacggcgaccaccgagatctacactctttccctacacgacgctcttccgatctacttgcaatcacagaaagtcttgtgcc

SEQ ID NO: 152 - DNA, 84 bp, synthetic construct
  source 1..84: mol_type = other DNA
aatgatacggcgaccaccgagatctacactctttccctacacgacgctcttccgatctctggggagaatatgaagtcgtgtccc

SEQ ID NO: 153 - DNA, 80 bp, synthetic construct
  source 1..80: mol_type = other DNA
aatgatacggcgaccaccgagatctacactctttccctacacgacgctcttccgatctgcttcaccaccagctgagttcc

SEQ ID NO: 154 - DNA, 79 bp, synthetic construct
  source 1..79: mol_type = other DNA
aatgatacggcgaccaccgagatctacactctttccctacacgacgctcttccgatctctggacagcaagcagagtgcc

SEQ ID NO: 155 - DNA, 90 bp, synthetic construct
  source 1..90: mol_type = other DNA
aatgatacggcgaccaccgagatctacactctttccctacacgacgctcttccgatctgacttacctggctttataattagcttggtccc

SEQ ID NO: 156 - DNA, 90 bp, synthetic construct
  source 1..90: mol_type = other DNA
aatgatacggcgaccaccgagatctacactctttccctacacgacgctcttccgatcttacttacttggaaagacttgtaatctggtccc

SEQ ID NO: 157 - DNA, 88 bp, synthetic construct
  source 1..88: mol_type = other DNA
aatgatacggcgaccaccgagatctacactctttccctacacgacgctcttccgatctcttacgtggtaaaacaatcacttgagtgcc

SEQ ID NO: 158 - DNA, 83 bp, synthetic construct
  source 1..83: mol_type = other DNA
aatgatacggcgaccaccgagatctacactctttccctacacgacgctcttccgatctggggaataacggtgagtctcgttcc

SEQ ID NO: 159 - DNA, 89 bp, synthetic construct
  source 1..89: mol_type = other DNA
aatgatacggcgaccaccgagatctacactctttccctacacgacgctcttccgatctcctacctggttttacttggtaaagttgtccc

SEQ ID NO: 160 - DNA, 82 bp, synthetic construct
  source 1..82: mol_type = other DNA
aatgatacggcgaccaccgagatctacactctttccctacacgacgctcttccgatctcggatttactgccaggcttgttcc

SEQ ID NO: 161 - DNA, 84 bp, synthetic construct
  source 1..84: mol_type = other DNA
aatgatacggcgaccaccgagatctacactctttccctacacgacgctcttccgatctcggggtttgaccattaaccttgttcc

SEQ ID NO: 162 - DNA, 81 bp, synthetic construct
  source 1..81: mol_type = other DNA
aatgatacggcgaccaccgagatctacactctttccctacacgacgctcttccgatctgctaaaaccttcagcctggtgcc

SEQ ID NO: 163 - DNA, 78 bp, synthetic construct
  source 1..78: mol_type = other DNA
aatgatacggcgaccaccgagatctacactctttccctacacgacgctcttccgatctgtgaccaacagcgaggtgcc

SEQ ID NO: 164 - DNA, 91 bp, synthetic construct
  source 1..91: mol_type = other DNA
aatgatacggcgaccaccgagatctacactctttccctacacgacgctcttccgatctacacttacttggtttaacagagagtttagtgcc

SEQ ID NO: 165 - DNA, 85 bp, synthetic construct
  source 1..85: mol_type = other DNA
aatgatacggcgaccaccgagatctacactctttccctacacgacgctcttccgatctacttggttttactgtcagtctggtccc

SEQ ID NO: 166 - DNA, 83 bp, synthetic construct
  source 1..83: mol_type = other DNA
aatgatacggcgaccaccgagatctacactctttccctacacgacgctcttccgatctcgagcgtgacctgaagtcttgttcc

SEQ ID NO: 167 - DNA, 84 bp, synthetic construct
  source 1..84: mol_type = other DNA
aatgatacggcgaccaccgagatctacactctttccctacacgacgctcttccgatctcagggctggatgattagatgagtccc

SEQ ID NO: 168 - DNA, 82 bp, synthetic construct
  source 1..82: mol_type = other DNA
aatgatacggcgaccaccgagatctacactctttccctacacgacgctcttccgatctgggcctaactgctaaacgagtccc

SEQ ID NO: 169 - DNA, 86 bp, synthetic construct
  source 1..86: mol_type = other DNA
aatgatacggcgaccaccgagatctacactctttccctacacgacgctcttccgatctcacaggacttgactctcagaatggttcc

SEQ ID NO: 170 - DNA, 85 bp, synthetic construct
  source 1..85: mol_type = other DNA
aatgatacggcgaccaccgagatctacactctttccctacacgacgctcttccgatctactgggtatgatggtgagtcttgttcc

SEQ ID NO: 171 - DNA, 84 bp, synthetic construct
  source 1..84: mol_type = other DNA
aatgatacggcgaccaccgagatctacactctttccctacacgacgctcttccgatctcttggaatgaccgtcaaacttgtccc

SEQ ID NO: 172 - DNA, 89 bp, synthetic construct
  source 1..89: mol_type = other DNA
aatgatacggcgaccaccgagatctacactctttccctacacgacgctcttccgatctacttacttggaatgactgataagcttgtccc

SEQ ID NO: 173 - DNA, 86 bp, synthetic construct
  source 1..86: mol_type = other DNA
aatgatacggcgaccaccgagatctacactctttccctacacgacgctcttccgatcttgcttggcttcacagttagtcatgtctc

SEQ ID NO: 174 - DNA, 84 bp, synthetic construct
  source 1..84: mol_type = other DNA
aatgatacggcgaccaccgagatctacactctttccctacacgacgctcttccgatctcttggatggacagtcaagatggtccc

SEQ ID NO: 175 - DNA, 90 bp, synthetic construct
  source 1..90: mol_type = other DNA
aatgatacggcgaccaccgagatctacactctttccctacacgacgctcttccgatctaacttacttggattcacggttaagagagttcc

SEQ ID NO: 176 - DNA, 83 bp, synthetic construct
  source 1..83: mol_type = other DNA
aatgatacggcgaccaccgagatctacactctttccctacacgacgctcttccgatctttgggttgatagtcagcctggttcc

SEQ ID NO: 177 - DNA, 94 bp, synthetic construct
  source 1..94: mol_type = other DNA
aatgatacggcgaccaccgagatctacactctttccctacacgacgctcttccgatctccacacttacttggatttatttttgtactcatcccc

SEQ ID NO: 178 - DNA, 93 bp, synthetic construct
  source 1..93: mol_type = other DNA
aatgatacggcgaccaccgagatctacactctttccctacacgacgctcttccgatcttaaaacatacctggtctaacactcagagttattcc

SEQ ID NO: 179 - DNA, 86 bp, synthetic construct
  source 1..86: mol_type = other DNA
aatgatacggcgaccaccgagatctacactctttccctacacgacgctcttccgatcttacatgggtttactgtcagtttcgttcc

SEQ ID NO: 180 - DNA, 84 bp, synthetic construct
  source 1..84: mol_type = other DNA
aatgatacggcgaccaccgagatctacactctttccctacacgacgctcttccgatctccaggattcactgtgagctgtgttcc

SEQ ID NO: 181 - DNA, 81 bp, synthetic construct
  source 1..81: mol_type = other DNA
aatgatacggcgaccaccgagatctacactctttccctacacgacgctcttccgatctagcttcactctcacttgcgtccc

SEQ ID NO: 182 - DNA, 84 bp, synthetic construct
  source 1..84: mol_type = other DNA
aatgatacggcgaccaccgagatctacactctttccctacacgacgctcttccgatctcccaggctcacaattaactcagtccc

SEQ ID NO: 183 - DNA, 90 bp, synthetic construct
  source 1..90: mol_type = other DNA
aatgatacggcgaccaccgagatctacactctttccctacacgacgctcttccgatcttacttgctgagtttcatgattcctctagtgtt

SEQ ID NO: 184 - DNA, 80 bp, synthetic construct
  source 1..80: mol_type = other DNA
aatgatacggcgaccaccgagatctacactctttccctacacgacgctcttccgatctgttccacagtcacacgggttcc

SEQ ID NO: 185 - DNA, 83 bp, synthetic construct
  source 1..83: mol_type = other DNA
aatgatacggcgaccaccgagatctacactctttccctacacgacgctcttccgatctctggttccacgatgagttgtgttcc

SEQ ID NO: 186 - DNA, 81 bp, synthetic construct
  source 1..81: mol_type = other DNA
aatgatacggcgaccaccgagatctacactctttccctacacgacgctcttccgatctggctccacgaagagtttgatgcc

SEQ ID NO: 187 - DNA, 88 bp, synthetic construct
  source 1..88: mol_type = other DNA
aatgatacggcgaccaccgagatctacactctttccctacacgacgctcttccgatctcttacgttgttgtacctccagataggttcc

SEQ ID NO: 188 - DNA, 87 bp, synthetic construct
  source 1..87: mol_type = other DNA
aatgatacggcgaccaccgagatctacactctttccctacacgacgctcttccgatctgtcttacctacaactgtgagtctggtgcc

SEQ ID NO: 189 - DNA, 86 bp, synthetic construct
  source 1..86: mol_type = other DNA
aatgatacggcgaccaccgagatctacactctttccctacacgacgctcttccgatctccttacctacaacggttaacctggtccc

SEQ ID NO: 190 - DNA, 89 bp, synthetic construct
  source 1..89: mol_type = other DNA
aatgatacggcgaccaccgagatctacactctttccctacacgacgctcttccgatctcttactcacctacaacagtgagccaacttcc

SEQ ID NO: 191 - DNA, 84 bp, synthetic construct
  source 1..84: mol_type = other DNA
aatgatacggcgaccaccgagatctacactctttccctacacgacgctcttccgatctatacccaagacagagagctgggttcc

SEQ ID NO: 192 - DNA, 87 bp, synthetic construct
  source 1..87: mol_type = other DNA
aatgatacggcgaccaccgagatctacactctttccctacacgacgctcttccgatctaacttacctaggatggagagtcgagtccc

SEQ ID NO: 193 - DNA, 80 bp, synthetic construct
  source 1..80: mol_type = other DNA
aatgatacggcgaccaccgagatctacactctttccctacacgacgctcttccgatctctgtcacagtgagcctggtccc

SEQ ID NO: 194 - DNA, 76 bp, synthetic construct
  source 1..76: mol_type = other DNA
aatgatacggcgaccaccgagatctacactctttccctacacgacgctcttccgatctcacggtgagccgtgtccc

SEQ ID NO: 195 - DNA, 80 bp, synthetic construct
  source 1..80: mol_type = other DNA
aatgatacggcgaccaccgagatctacactctttccctacacgacgctcttccgatctccagtacggtcagcctagagcc

SEQ ID NO: 196 - DNA, 76 bp, synthetic construct
  source 1..76: mol_type = other DNA
aatgatacggcgaccaccgagatctacactctttccctacacgacgctcttccgatctcactgtcagccgggtgcc

SEQ ID NO: 197 - DNA, 76 bp, synthetic construct
  source 1..76: mol_type = other DNA
aatgatacggcgaccaccgagatctacactctttccctacacgacgctcttccgatctcactgagagccgggtccc

SEQ ID NO: 198 - DNA, 75 bp, synthetic construct
  source 1..75: mol_type = other DNA
aatgatacggcgaccaccgagatctacactctttccctacacgacgctcttccgatctaccaggagccgcgtgcc

SEQ ID NO: 199 - DNA, 76 bp, synthetic construct
  source 1..76: mol_type = other DNA
aatgatacggcgaccaccgagatctacactctttccctacacgacgctcttccgatctcacggtcagcctgctgcc

SEQ ID NO: 200 - DNA, 76 bp, synthetic construct
  source 1..76: mol_type = other DNA
aatgatacggcgaccaccgagatctacactctttccctacacgacgctcttccgatctgaccgtgagcctggtgcc

SEQ ID NO: 201 - DNA, 92 bp, synthetic construct
  source 1..92: mol_type = other DNA
aatgatacggcgaccaccgagatctacactctttccctacacgacgctcttccgatctgtgaagttactatgagcttagtcccttcagcaaa

SEQ ID NO: 202 - DNA, 91 bp, synthetic construct
  source 1..91: mol_type = other DNA
aatgatacggcgaccaccgagatctacactctttccctacacgacgctcttccgatctcgaagttactatgagcctagtcccttttgcaaa

SEQ ID NO: 203 - DNA, 86 bp, synthetic construct
  source 1..86: mol_type = other DNA
aatgatacggcgaccaccgagatctacactctttccctacacgacgctcttccgatcttgacaacaagtgttgttccactgccaaa

SEQ ID NO: 204 - DNA, 89 bp, synthetic construct
  source 1..89: mol_type = other DNA
aatgatacggcgaccaccgagatctacactctttccctacacgacgctcttccgatctctgtaatgataagctttgttccgggaccaaa

SEQ ID NO: 205 - DNA, 30 bp, synthetic construct
  source 1..30: mol_type = other DNA
gatggagtcgggaaggaagtcctgtgcgag

SEQ ID NO: 206 - DNA, 25 bp, synthetic construct
  source 1..25: mol_type = other DNA
gggaagacsgatgggcccttggtgg

SEQ ID NO: 207 - DNA, 27 bp, synthetic construct
  source 1..27: mol_type = other DNA
caggcakgcgaygaccacgttcccatc

SEQ ID NO: 208 - DNA, 20 bp, synthetic construct
  source 1..20: mol_type = other DNA
ccacagggctgttatccttt

SEQ ID NO: 209 - DNA, 20 bp, synthetic construct
  source 1..20: mol_type = other DNA
agggaatgtttttgcagcag

SEQ ID NO: 210 - DNA, 34 bp, synthetic construct
  source 1..34: mol_type = other DNA
catcagatggcgggaagatgaagacagatggtgc

SEQ ID NO: 211 - DNA, 28 bp, synthetic construct
  source 1..28: mol_type = other DNA
cctcagaggagggtgggaacagagtgac

SEQ ID NO: 212 - DNA, 31 bp, synthetic construct
  source 1..31: mol_type = other DNA
gctcaaacacagcgacctcgggtgggaacac

SEQ ID NO: 213 - DNA, 30 bp, synthetic construct
  source 1..30: mol_type = other DNA
tctctcagctggtacacggcagggtcaggg

SEQ ID NO: 214 - DNA, 24 bp, synthetic construct
  source 1..24: mol_type = other DNA
ggtagcacatactacgcagactcc

SEQ ID NO: 215 - DNA, 25 bp, synthetic construct
  source 1..25: mol_type = other DNA
ggacaacagaatagaccacgtctgt

SEQ ID NO: 216 - DNA, 25 bp, synthetic construct
  source 1..25: mol_type = other DNA
acaacgccaataactcaccgtatct

SEQ ID NO: 217 - DNA, 26 bp, synthetic construct
  source 1..26: mol_type = other DNA
ggagcacaaattacaacccacttctc

SEQ ID NO: 218 - DNA, 19 bp, synthetic construct
  source 1..19: mol_type = other DNA
gggctgcccaaagatcggt

SEQ ID NO: 219 - DNA, 25 bp, synthetic construct
  source 1..25: mol_type = other DNA
cagagacaattccaagaacacgctg

SEQ ID NO: 220 - DNA, 19 bp, synthetic construct
  source 1..19: mol_type = other DNA
tgtcagcacggcgtgtctt

SEQ ID NO: 221 - DNA, 19 bp, synthetic construct
  source 1..19: mol_type = other DNA
agggcgccagttccatgac

SEQ ID NO: 222 - DNA, 25 bp, synthetic construct
  source 1..25: mol_type = other DNA
acacccctgataacttccaatccag

SEQ ID NO: 223 - DNA, 25 bp, synthetic construct
  source 1..25: mol_type = other DNA
cagtggtaacacaaactacgcacag

SEQ ID NO: 224 - DNA, 27 bp, synthetic construct
  source 1..27: mol_type = other DNA
cctgatgtatcattctccactctgagg

SEQ ID NO: 225 - DNA, 23 bp, synthetic construct
  source 1..23: mol_type = other DNA
gattcacttgcagtaacgcctgg

SEQ ID NO: 226 - DNA, 26 bp, synthetic construct
  source 1..26: mol_type = other DNA
gagggatctactctggcaatggtaag

SEQ ID NO: 227 - DNA, 27 bp, synthetic construct
  source 1..27: mol_type = other DNA
caacaacgttccgatagatgattcagg

SEQ ID NO: 228 - DNA, 24 bp, synthetic construct
  source 1..24: mol_type = other DNA
tactccgttggtattggccagatc

SEQ ID NO: 229 - DNA, 26 bp, synthetic construct
  source 1..26: mol_type = other DNA
cagagtgatgctcaacgagacaaatc

SEQ ID NO: 230 - DNA, 19 bp, synthetic construct
  source 1..19: mol_type = other DNA
tctgtcagcacggcggatc

SEQ ID NO: 231 - DNA, 27 bp, synthetic construct
  source 1..27: mol_type = other DNA
tcaccagttccctaactatagctctga

SEQ ID NO: 232 - DNA, 26 bp, synthetic construct
  source 1..26: mol_type = other DNA
cagttgcctaaggatcgattttctgc

SEQ ID NO: 233 - DNA, 26 bp, synthetic construct
  source 1..26: mol_type = other DNA
ctgacagctctcgcttataccttcat

SEQ ID NO: 234 - DNA, 25 bp, synthetic construct
  source 1..25: mol_type = other DNA
ctctagagagaagaaggagcgcttc

SEQ ID NO: 235 - DNA, 27 bp, synthetic construct
  source 1..27: mol_type = other DNA
tgaagggtacaaagtctctcgaaaaga

SEQ ID NO: 236 - DNA, 27 bp, synthetic construct
  source 1..27: mol_type = other DNA
gcaacagtgagttatcagggttactct

SEQ ID NO: 237 - DNA, 27 bp, synthetic construct
  source 1..27: mol_type = other DNA
aattgtatctgcttgcccctagaagat

SEQ ID NO: 238 - DNA, 27 bp, synthetic construct
  source 1..27: mol_type = other DNA
cgctggcaatggtaacacaaaatattc

SEQ ID NO: 239 - DNA, 27 bp, synthetic construct
  source 1..27: mol_type = other DNA
gtgaaacaatctacgcacagaagttcc

SEQ ID NO: 240 - DNA, 19 bp, synthetic construct
  source 1..19: mol_type = other DNA
cacctctgtcagcacggca

SEQ ID NO: 241 - DNA, 24 bp, synthetic construct
  source 1..24: mol_type = other DNA
gtaggacgcactatgcagactctg

SEQ ID NO: 242 - DNA, 21 bp, synthetic construct
  source 1..21: mol_type = other DNA
ccacagaggatttcccgctca

SEQ ID NO: 243 - DNA, 26 bp, synthetic construct
  source 1..26: mol_type = other DNA
gtggtgataccgtactctacacagac

SEQ ID NO: 244 - DNA, 25 bp, synthetic construct
  source 1..25: mol_type = other DNA
ccctcaagagtcgagtcaccatatc

SEQ ID NO: 245 - DNA, 27 bp, synthetic construct
  source 1..27: mol_type = other DNA
ggggacaacacttaacatcacaatctc

SEQ ID NO: 246 - DNA, 19 bp, synthetic construct
  source 1..19: mol_type = other DNA
gtgtcctccccacaggtgc

SEQ ID NO: 247 - DNA, 27 bp, synthetic construct
  source 1..27: mol_type = other DNA
ttcacgacactaacaaaggagaagtct

SEQ ID NO: 248 - DNA, 27 bp, synthetic construct
  source 1..27: mol_type = other DNA
gtaagacaggctatgcacagaagtttc

SEQ ID NO: 249 - DNA, 27 bp, synthetic construct
  source 1..27: mol_type = other DNA
gtagcacatactatgcagactctgtga

SEQ ID NO: 250 - DNA, 19 bp, synthetic construct
  source 1..19: mol_type = other DNA
tcgattctcagggcgccag

SEQ ID NO: 251 - DNA, 23 bp, synthetic construct
  source 1..23: mol_type = other DNA
ccaagtcgcttctcacctgaatg

SEQ ID NO: 252 - DNA, 21 bp, synthetic construct
  source 1..21: mol_type = other DNA
ccatctccagagacaacgcca

SEQ ID NO: 253 - DNA, 27 bp, synthetic construct
  source 1..27: mol_type = other DNA
agttccaaatcgcttctcacctaaatc

SEQ ID NO: 254 - DNA, 26 bp, synthetic construct
  source 1..26: mol_type = other DNA
tggcacaaactatgcacagaagtttc

SEQ ID NO: 255 - DNA, 27 bp, synthetic construct
  source 1..27: mol_type = other DNA
atgttatgcacaatctgtgaagagcag

SEQ ID NO: 256 - DNA, 27 bp, synthetic construct
  source 1..27: mol_type = other DNA
caaggaaagattttcagctaagtgcct

SEQ ID NO: 257 - DNA, 25 bp, synthetic construct
  source 1..25: mol_type = other DNA
aacattcttgaacgattctccgcac

SEQ ID NO: 258 - DNA, 25 bp, synthetic construct
  source 1..25: mol_type = other DNA
cctgcaagacttgtggatacaccta

SEQ ID NO: 259 - DNA, 23 bp, synthetic construct
  source 1..23: mol_type = other DNA
agtcaagtgagacttcacgcact

SEQ ID NO: 260 - DNA, 21 bp, synthetic construct
  source 1..21: mol_type = other DNA
cgagcagattcaccgtctcca

SEQ ID NO: 261 - DNA, 27 bp, synthetic construct
  source 1..27: mol_type = other DNA
aggacgtttgtgtattttcaggtgttc

SEQ ID NO: 262 - DNA, 27 bp, synthetic construct
  source 1..27: mol_type = other DNA
cggttgatctattgctcctttgatgtc

SEQ ID NO: 263 - DNA, 20 bp, synthetic construct
  source 1..20: mol_type = other DNA
ttgtacagcccagcggttca

SEQ ID NO: 264 - DNA, 21 bp, synthetic construct
  source 1..21: mol_type = other DNA
agcagacaaatcggggcttcc

SEQ ID NO: 265 - DNA, 25 bp, synthetic construct
  source 1..25: mol_type = other DNA
ctccagatccatgtccaaaaagcag

SEQ ID NO: 266 - DNA, 26 bp, synthetic construct
  source 1..26: mol_type = other DNA
ctgagtcagcatagtcattccaacca

SEQ ID NO: 267 - DNA, 27 bp, synthetic construct
  source 1..27: mol_type = other DNA
agagacaatgccaagaactccttgtat

SEQ ID NO: 268 - DNA, 27 bp, synthetic construct
  source 1..27: mol_type = other DNA
gataaaggaagcatccctgatcgattc

SEQ ID NO: 269 - DNA, 27 bp, synthetic construct
  source 1..27: mol_type = other DNA
aatgataacacacactacgcacagaag

SEQ ID NO: 270 - DNA, 27 bp, synthetic construct
  source 1..27: mol_type = other DNA
caatgtctccagattaaacaaacggga

SEQ ID NO: 271 - DNA, 20 bp, synthetic construct
  source 1..20: mol_type = other DNA
agtccccgatggctacgttg

SEQ ID NO: 272 - DNA, 27 bp, synthetic construct
  source 1..27: mol_type = other DNA
cacaagaagcgattctcatctcaatgc

SEQ ID NO: 273 - DNA, 27 bp, synthetic construct
  source 1..27: mol_type = other DNA
aatgacaaaaaatcctacagcacgtct

SEQ ID NO: 274 - DNA, 27 bp, synthetic construct
  source 1..27: mol_type = other DNA
gtagtagtggttgtagcacaaactacg

SEQ ID NO: 275 - DNA, 27 bp, synthetic construct
  source 1..27: mol_type = other DNA
gtaacaccaactacgcacagaaattcc

SEQ ID NO: 276 - DNA, 20 bp, synthetic construct
  source 1..20: mol_type = other DNA
tacgccgcgtctgtgaaagg

SEQ ID NO: 277 - DNA, 27 bp, synthetic construct
  source 1..27: mol_type = other DNA
ctccagatctatgtccaaaaacagctc

SEQ ID NO: 278 - DNA, 19 bp, synthetic construct
  source 1..19: mol_type = other DNA
ggtacagcttggggggtcc

SEQ ID NO: 279 - DNA, 20 bp, synthetic construct
  source 1..20: mol_type = other DNA
gcataggtcacgagggagca

SEQ ID NO: 280 - DNA, 20 bp, synthetic construct
  source 1..20: mol_type = other DNA
agtccatcagcaccgcctac

SEQ ID NO: 281 - DNA, 22 bp, synthetic construct
  source 1..22: mol_type = other DNA
tggtttcgggtttactgggtgc

SEQ ID NO: 282 - DNA, 27 bp, synthetic construct
  source 1..27: mol_type = other DNA
aatgtctccagatcaaacacagaggat

SEQ ID NO: 283 - DNA, 20 bp, synthetic construct
  source 1..20: mol_type = other DNA
ggctgcccaatgatcggttc

SEQ ID NO: 284 - DNA, 27 bp, synthetic construct
  source 1..27: mol_type = other DNA
ctcatttgcagcttctagattcacctt

SEQ ID NO: 285 - DNA, 27 bp, synthetic construct
  source 1..27: mol_type = other DNA
ctccagaataaggatagagcgttttcc

SEQ ID NO: 286 - DNA, 27 bp, synthetic construct
  source 1..27: mol_type = other DNA
gaacagtgttctgatatcgacaagacc

SEQ ID NO: 287 - DNA, 27 bp, synthetic construct
  source 1..27: mol_type = other DNA
ggaaacttgcctaattgattctcagct

SEQ ID NO: 288 - DNA, 27 bp, synthetic construct
  source 1..27: mol_type = other DNA
tccttctccagagattcatccaagaaa

SEQ ID NO: 289 - DNA, 26 bp, synthetic construct
  source 1..26: mol_type = other DNA
gtctccagaataaggacggagcattt

SEQ ID NO: 290 - DNA, 27 bp, synthetic construct
  source 1..27: mol_type = other DNA
atatggcgtggtgaaagtcatcaatac

SEQ ID NO: 291 - DNA, 19 bp, synthetic construct
  source 1..19: mol_type = other DNA
ggctgcccagtgatcgctt

SEQ ID NO: 292 - DNA, 26 bp, synthetic construct
  source 1..26: mol_type = other DNA
gagatctaactgaaggctacgtgtct

SEQ ID NO: 293 - DNA, 25 bp, synthetic construct
  source 1..25: mol_type = other DNA
ttgagtggatggaacgtgttgatcc

SEQ ID NO: 294 - DNA, 27 bp, synthetic construct
  source 1..27: mol_type = other DNA
tggtaacaccaactatgcaaagaagtt

SEQ ID NO: 295 - DNA, 27 bp, synthetic construct
  source 1..27: mol_type = other DNA
cataggtcatgaagggagcacacatta

SEQ ID NO: 296 - DNA, 24 bp, synthetic construct
  source 1..24: mol_type = other DNA
gtgacagaacagtggctatgtgtg

SEQ ID NO: 297 - DNA, 19 bp, synthetic construct
  source 1..19: mol_type = other DNA
cctgagactctgccgtgca

SEQ ID NO: 298 - DNA, 19 bp, synthetic construct
  source 1..19: mol_type = other DNA
cagggcttcacaggacggt

SEQ ID NO: 299 - DNA, 27 bp, synthetic construct
  source 1..27: mol_type = other DNA
ctccagagacaattctaagagcatgct

SEQ ID NO: 300 - DNA, 20 bp, synthetic construct
  source 1..20: mol_type = other DNA
gccgagttcaccagtctcca

SEQ ID NO: 301 - DNA, 20 bp, synthetic construct
  source 1..20: mol_type = other DNA
ccacatacgagcaaggcgtc

SEQ ID NO: 302 - DNA, 26 bp, synthetic construct
  source 1..26: mol_type = other DNA
gccaaaggaacgattttctgctgaat

SEQ ID NO: 303 - DNA, 18 bp, synthetic construct
  source 1..18: mol_type = other DNA
gcaaagcctgcgtggtcc

SEQ ID NO: 304 - DNA, 18 bp, synthetic construct
  source 1..18: mol_type = other DNA
cgctgcacccgtgaaagg

SEQ ID NO: 305 - DNA, 27 bp, synthetic construct
  source 1..27: mol_type = other DNA
cagtaccaaaacattgcagttgattca

SEQ ID NO: 306 - DNA, 27 bp, synthetic construct
  source 1..27: mol_type = other DNA
actacatcacaaacagtgcttatgact

SEQ ID NO: 307 - DNA, 27 bp, synthetic construct
  source 1..27: mol_type = other DNA
agaaaggagatatagctgaagggtaca

SEQ ID NO: 308 - DNA, 20 bp, synthetic construct
  source 1..20: mol_type = other DNA
accaacggcttcacaggacg

SEQ ID NO: 309 - DNA, 25 bp, synthetic construct
  source 1..25: mol_type = other DNA
tgcttctccattacaaccagtgctt

SEQ ID NO: 310 - DNA, 27 bp, synthetic construct
  source 1..27: mol_type = other DNA
aaaggagagatctctgatggatacagt

SEQ ID NO: 311 - DNA, 19 bp, synthetic construct
  source 1..19: mol_type = other DNA
ctccgtgaagcgccgattc

SEQ ID NO: 312 - DNA, 27 bp, synthetic construct
  source 1..27: mol_type = other DNA
tctccagttccctaattatagctctga

SEQ ID NO: 313 - DNA, 27 bp, synthetic construct
  source 1..27: mol_type = other DNA
catctccagatccatgtccaaaaagta

SEQ ID NO: 314 - DNA, 19 bp, synthetic construct
  source 1..19: mol_type = other DNA
ggtgcagctgcaggagtgg

SEQ ID NO: 315 - DNA, 27 bp, synthetic construct
  source 1..27: mol_type = other DNA
ggtaacacaacatatgcacagaagttc

SEQ ID NO: 316 - DNA, 27 bp, synthetic construct
  source 1..27: mol_type = other DNA
caacaatcgattcttagctgaaaggac

SEQ ID NO: 317 - DNA, 24 bp, synthetic construct
  source 1..24: mol_type = other DNA
agctcgccagttccctaactatag

SEQ ID NO: 318 - DNA, 25 bp, synthetic construct
  source 1..25: mol_type = other DNA
cacatactatgcagactccgtgaag

SEQ ID NO: 319 - DNA, 27 bp, synthetic construct
  source 1..27: mol_type = other DNA
agctaacagttacacgacagaatatgc

SEQ ID NO: 320 - DNA, 27 bp, synthetic construct
  source 1..27: mol_type = other DNA
gtcagacagagaaatactacagaccag

SEQ ID NO: 321 - DNA, 27 bp, synthetic construct
  source 1..27: mol_type = other DNA
actcgccttcagtacaaagaagattaa

SEQ ID NO: 322 - DNA, 27 bp, synthetic construct
  source 1..27: mol_type = other DNA
aagtgggagttctcagagttactctcc

SEQ ID NO: 323 - DNA, 20 bp, synthetic construct
  source 1..20: mol_type = other DNA
gtcaccataaccgcggacac

SEQ ID NO: 324 - DNA, 18 bp, synthetic construct
  source 1..18: mol_type = other DNA
agggacacgtccacgagc

SEQ ID NO: 325 - DNA, 27 bp, synthetic construct
  source 1..27: mol_type = other DNA
ccaaacctaacattctcaactctgact

SEQ ID NO: 326 - DNA, 18 bp, synthetic construct
  source 1..18: mol_type = other DNA
catccatcaccccccgca

SEQ ID NO: 327 - DNA, 27 bp, synthetic construct
  source 1..27: mol_type = other DNA
gatgatggaagtcagatacaccatgca

SEQ ID NO: 328 - DNA, 27 bp, synthetic construct
  source 1..27: mol_type = other DNA
gggtcccagttattagtggtagtggta

SEQ ID NO: 329 - DNA, 27 bp, synthetic construct
  source 1..27: mol_type = other DNA
aaagtgtgacggaagtgagaaatacta

SEQ ID NO: 330 - DNA, 27 bp, synthetic construct
  source 1..27: mol_type = other DNA
gagtgatcaagtatgaattctcagggt

SEQ ID NO: 331 - DNA, 27 bp, synthetic construct
  source 1..27: mol_type = other DNA
cctgatggatcattctctactctgaag

SEQ ID NO: 332 - DNA, 27 bp, synthetic construct
  source 1..27: mol_type = other DNA
gaatgggtctcatacattagtgctagt

SEQ ID NO: 333 - DNA, 20 bp, synthetic construct
  source 1..20: mol_type = other DNA
ggctccgaggtcacatacga

SEQ ID NO: 334 - DNA, 23 bp, synthetic construct
  source 1..23: mol_type = other DNA
tcgaatcaccatgtccgtagaca

SEQ ID NO: 335 - DNA, 22 bp, synthetic construct
  source 1..22: mol_type = other DNA
tgcttctccatcacaaccagtg

SEQ ID NO: 336 - DNA, 27 bp, synthetic construct
  source 1..27: mol_type = other DNA
gctgctggtactactgacaaagaagtc

SEQ ID NO: 337 - DNA, 27 bp, synthetic construct
  source 1..27: mol_type = other DNA
ccttccaggatgaaactcaacaagata

SEQ ID NO: 338 - DNA, 20 bp, synthetic construct
  source 1..20: mol_type = other DNA
cacgattaccgcggacgaat

SEQ ID NO: 339 - DNA, 27 bp, synthetic construct
  source 1..27: mol_type = other DNA
gagatgtctctgagaggtatcatgttt

SEQ ID NO: 340 - DNA, 20 bp, synthetic construct
  source 1..20: mol_type = other DNA
ctggagtgggttggccgtat

SEQ ID NO: 341 - DNA, 27 bp, synthetic construct
  source 1..27: mol_type = other DNA
atttattgggatgatgataagcgctac

SEQ ID NO: 342 - DNA, 27 bp, synthetic construct
  source 1..27: mol_type = other DNA
agaaagcagaaataatcaatgagcgat

SEQ ID NO: 343 - DNA, 26 bp, synthetic construct
  source 1..26: mol_type = other DNA
gcctgatggatcaaatttcactctga

SEQ ID NO: 344 - DNA, 27 bp, synthetic construct
  source 1..27: mol_type = other DNA
acagtgtgatggaagtcagatatgtta

SEQ ID NO: 345 - DNA, 27 bp, synthetic construct
  source 1..27: mol_type = other DNA
ctgagactgatctattactcaagggtt

SEQ ID NO: 346 - DNA, 27 bp, synthetic construct
  source 1..27: mol_type = other DNA
ccctgtctctatttgatcatccatttt

SEQ ID NO: 347 - DNA, 27 bp, synthetic construct
  source 1..27: mol_type = other DNA
gccgactcattattcagttaacattga

SEQ ID NO: 348 - DNA, 27 bp, synthetic construct
  source 1..27: mol_type = other DNA
gatggctacaatgtctccagattaaaa

SEQ ID NO: 349 - DNA, 27 bp, synthetic construct
  source 1..27: mol_type = other DNA
ccattattcaaatactgcaggtaccac

SEQ ID NO: 350 - DNA, 27 bp, synthetic construct
  source 1..27: mol_type = other DNA
caattatgaagctcaaccagacaaatc

SEQ ID NO: 351 - DNA, 27 bp, synthetic construct
  source 1..27: mol_type = other DNA
cttacttccagaatgaagctcaactag

SEQ ID NO: 352 - DNA, 27 bp, synthetic construct
  source 1..27: mol_type = other DNA
tggtgttaaagatactgacaaaggaga

SEQ ID NO: 353 - DNA, 27 bp, synthetic construct
  source 1..27: mol_type = other DNA
acacttacaatggtaacacaaactacc

SEQ ID NO: 354 - DNA, 27 bp, synthetic construct
  source 1..27: mol_type = other DNA
tcatttatgagttgttgtgtaggtagc

SEQ ID NO: 355 - DNA, 27 bp, synthetic construct
  source 1..27: mol_type = other DNA
gtctcatgtattagtactgatgggagt

SEQ ID NO: 356 - DNA, 18 bp, synthetic construct
  source 1..18: mol_type = other DNA
gccctctgggaaggcgct

SEQ ID NO: 357 - DNA, 27 bp, synthetic construct
  source 1..27: mol_type = other DNA
ggatttccaatcataaccagtacttcc

SEQ ID NO: 358 - DNA, 27 bp, synthetic construct
  source 1..27: mol_type = other DNA
acactttacagacaccatcaattttcc

SEQ ID NO: 359 - DNA, 27 bp, synthetic construct
  source 1..27: mol_type = other DNA
accctccatcaatacaaagaaaaatca

SEQ ID NO: 360 - DNA, 27 bp, synthetic construct
  source 1..27: mol_type = other DNA
gtgatatgggttaagggaaacactaag

SEQ ID NO: 361 - DNA, 27 bp, synthetic construct
  source 1..27: mol_type = other DNA
cgcttacaatggtaacacaaactatgc

SEQ ID NO: 362 - DNA, 27 bp, synthetic construct
  source 1..27: mol_type = other DNA
tgggatgatgataaatactacagcaca

SEQ ID NO: 363 - DNA, 27 bp, synthetic construct
  source 1..27: mol_type = other DNA
ttgggatgatgataaattctacagcac

SEQ ID NO: 364 - DNA, 27 bp, synthetic construct
  source 1..27: mol_type = other DNA
ctattatagtgggagcacctactacaa

SEQ ID NO: 365 - DNA, 27 bp, synthetic construct
  source 1..27: mol_type = other DNA
gtccaagtggtataatgattatgcagt

SEQ ID NO: 366 - DNA, 27 bp, synthetic construct
  source 1..27: mol_type = other DNA
tagactggatcatcaaggaatacacat

SEQ ID NO: 367 - DNA, 20 bp, synthetic construct
  source 1..20: mol_type = other DNA
gcacaagctacgcagactcc

SEQ ID NO: 368 - DNA, 22 bp, synthetic construct
  source 1..22: mol_type = other DNA
ggtgacccatactatccaggct

SEQ ID NO: 369 - DNA, 19 bp, synthetic construct
  source 1..19: mol_type = other DNA
agcatgaccaaaggcggtg

SEQ ID NO: 370 - DNA, 19 bp, synthetic construct
  source 1..19: mol_type = other DNA
gcaaccgggctcctctaga

SEQ ID NO: 371 - DNA, 19 bp, synthetic construct
  source 1..19: mol_type = other DNA
atgctgcgtcggtgaaagg

SEQ ID NO: 372 - DNA, 18 bp, synthetic construct
  source 1..18: mol_type = other DNA
ccttccaaggccacgtca

SEQ ID NO: 373 - DNA, 20 bp, synthetic construct
  source 1..20: mol_type = other DNA
aaccagggacacatccatgg

SEQ ID NO: 374 - DNA, 20 bp, synthetic construct
  source 1..20: mol_type = other DNA
tagtgatggcagcacaagct

SEQ ID NO: 375 - DNA, 21 bp, synthetic construct
  source 1..21: mol_type = other DNA
cacgattaccgcggacaaatc

SEQ ID NO: 376 - DNA, 23 bp, synthetic construct
  source 1..23: mol_type = other DNA
gtggttacacaaactacgcagac

SEQ ID NO: 377 - DNA, 21 bp, synthetic construct
  source 1..21: mol_type = other DNA
cggtaagacgcactatgtgga

SEQ ID NO: 378 - DNA, 24 bp, synthetic construct
  source 1..24: mol_type = other DNA
tcgaatgacgaaaaatcctacagc

SEQ ID NO: 379 - DNA, 25 bp, synthetic construct
  source 1..25: mol_type = other DNA
gtcaccgtctcttcaggtaagatgg

SEQ ID NO: 380 - DNA, 19 bp, synthetic construct
  source 1..19: mol_type = other DNA
ctcttgactcgggggtgcc

SEQ ID NO: 381 - DNA, 19 bp, synthetic construct
  source 1..19: mol_type = other DNA
gctcgggtttttgtgcggg

SEQ ID NO: 382 - DNA, 26 bp, synthetic construct
  source 1..26: mol_type = other DNA
tgttgtaggtgagtaagtcaaggctg

SEQ ID NO: 383 - DNA, 18 bp, synthetic construct
  source 1..18: mol_type = other DNA
gagtcccactgcagcccc

SEQ ID NO: 384 - DNA, 25 bp, synthetic construct
  source 1..25: mol_type = other DNA
cctcaggtaagaatggccactctag

SEQ ID NO: 385 - DNA, 19 bp, synthetic construct
  source 1..19: mol_type = other DNA
cgtcgcagggccagtttct

SEQ ID NO: 386 - DNA, 18 bp, synthetic construct
  source 1..18: mol_type = other DNA
agtcggagggtggaccgg

SEQ ID NO: 387 - DNA, 27 bp, synthetic construct
  source 1..27: mol_type = other DNA
ctctccatcctaggtaagttgcagaat

SEQ ID NO: 388 - DNA, 20 bp, synthetic construct
  source 1..20: mol_type = other DNA
ggtcaccgtctcctcaggtg

SEQ ID NO: 389 - DNA, 20 bp, synthetic construct
  source 1..20: mol_type = other DNA
gtgagttttcgcgggaccac

SEQ ID NO: 390 - DNA, 19 bp, synthetic construct
  source 1..19: mol_type = other DNA
tacgtgggaggccagcaga

SEQ ID NO: 391 - DNA, 24 bp, synthetic construct
  source 1..24: mol_type = other DNA
gttaaccgttgtaggtaaggctgg

SEQ ID NO: 392 - DNA, 19 bp, synthetic construct
  source 1..19: mol_type = other DNA
cacgaccccagaaccctgt

SEQ ID NO: 393 - DNA, 27 bp, synthetic construct
  source 1..27: mol_type = other DNA
gatagtgtatcataaggtcggagttcc

SEQ ID NO: 394 - DNA, 19 bp, synthetic construct
  source 1..19: mol_type = other DNA
tgggtaaggaggcggttgg

SEQ ID NO: 395 - DNA, 20 bp, synthetic construct
  source 1..20: mol_type = other DNA
ttgggtctggtttttgcggg

SEQ ID NO: 396 - DNA, 27 bp, synthetic construct
  source 1..27: mol_type = other DNA
tgtcccttttagagtggctatattctt

SEQ ID NO: 397 - DNA, 18 bp, synthetic construct
  source 1..18: mol_type = other DNA
ggtctcagcccgggggtc

SEQ ID NO: 398 - DNA, 18 bp, synthetic construct
  source 1..18: mol_type = other DNA
cctaccagccgcagggtt

SEQ ID NO: 399 - DNA, 19 bp, synthetic construct
  source 1..19: mol_type = other DNA
gcaccggtttttgtcctgg

(19) United States
(12) Patent Application Publication
(10) Pub. No.: US 2024/0150834 A1
(43) Pub. Date: May 9, 2024

(54) PRIMER EXTENSION TARGET ENRICHMENT OF IMMUNE RECEPTOR SEQUENCES IN ALLOIMMUNE DISORDERS OF PREGNANCY

(71) Applicant: Roche Sequencing Solutions, Inc., Pleasanton, CA (US)

(72) Inventors: Eva MARTIN, San Francisco, CA (US); Florian RUBELT, Menlo Park, CA (US)

(21) Appl. No.: 17/980,393

(22) Filed: Nov. 3, 2022

(51) Int. Cl. C12Q 1/6881; C12N 15/10; C12Q 1/6813; C12Q 1/6844

(52) U.S. Cl. CPC C12Q 1/6881; C12N 15/1065; C12Q 1/6813; C12Q 1/6844; C12Q 2600/112

(57) ABSTRACT

Methods of monitoring the development of FNAIT using immune profiling is described.

### BACKGROUND OF THE INVENTION

Alloimmune disorders of pregnancy result from maternal immunization to fetal blood cells and lead to fetal cytopenias. Discordance in blood cell antigens between the mother and fetus may cause the mother's immune system to mount antibodies against the fetal blood cell antigen. The attacked fetal blood cells can be red blood cells, resulting in Hemolytic Disease of the Fetus and Newborn (HDFN); platelets, resulting in Fetal and Neonatal Alloimmune Thrombocytopenia (FNAIT); or neutrophils, resulting in Alloimmune Neutropenia. Maternally derived antibodies reach the fetus via placental transfusion and cause destruction of the fetal blood cells. Clinical manifestations of the disorder arise from destruction of the affected fetal blood cells.

HDFN is the most common Alloimmune disorder of pregnancy, in which the mother is exposed to paternally-inherited fetal red blood cell antigens that she lacks, and the mother mounts an immune response against the fetal red blood cells. The most common red blood cell antigen causing HDFN is RhD, but discordance between the mother and fetus for other antigens such as RhC, RhE, Kelly, and Duffy are also associated with HDFN.

FNAIT occurs when a fetus carries a platelet antigen that the mother lacks and results in a maternal immune response to the fetus's platelets, causing potentially serious harm to the fetus. The most common cause of FNAIT in Caucasian populations is human platelet antigen-1a (HPA-1a). In this case, a fetus's platelets express paternally-inherited HPA-1a. The HPA-1a negative mother becomes sensitized to the fetal HPA-1a and mounts antibodies against the fetal platelets, resulting in the development of FNAIT. Alloimmunization to other human platelet antigens, such as HPA-5b, can cause FNAIT. Genotyping to determine the human platelet antigen status of fetuses and mothers could identify pregnancies with discordant genotypes that are at risk of FNAIT. Subsequent in vitro diagnostic tests to screen for pregnancies at highest risk of developing FNAIT, to monitor for the development of FNAIT, or to monitor the severity of known cases of FNAIT have not been described.

### BRIEF SUMMARY OF THE INVENTION

Methods and compositions are provided for quantifying immune specific genes in a pregnant woman that lacks an antigen carried by the fetus and can cause an alloimmune disorder in pregnancy (which can include but are not limited to FNAIT, HDFN or alloimmune neutropenia. In some embodiments, the disclosure provides a method of quantifying immune specific genes in a pregnant woman carrying a fetus encoding an alloimmune disorder causing antigen (e.g., HPA-1a antigen), wherein the pregnant woman lacks the antigen. In some embodiments, the method comprises:
a) providing a reaction mixture comprising:
i) a plurality of structurally different target polynucleotides from the pregnant woman, wherein individual target polynucleotides of the plurality comprise immune cell receptor V, J, and optionally C or D gene regions; and
ii) a plurality of immune cell receptor V gene specific primers, wherein the immune cell receptor V gene specific primers comprise at least 10 structurally distinct primers having the following regions from 5′ to 3′: [5′-Phos], [SPLINT], [BARCODE], and [FW], wherein:
[5′-Phos] comprises a 5′ phosphate;
[SPLINT] comprises an adaptor hybridization site of 2-8 nucleotides in length;
[BARCODE] comprises a barcode region of at least 6 nucleotides in length, wherein each nucleotide of the barcode region is independently selected from the group consisting of N and W; and
[FW] of each immune cell receptor V gene specific primer comprises a structurally distinct region that specifically hybridizes to a framework 1, framework 2, or framework 3 region of an immune cell receptor V gene, wherein the immune cell receptor V gene specific primers are hybridized to the V gene regions of the target polynucleotides;
b) extending the hybridized immune cell receptor V gene specific primers with a polymerase, and then removing un-extended immune cell receptor V gene specific primers, if present, wherein the extended immune cell receptor V gene specific primers comprise at least a portion of the immune cell receptor V region, optionally the immune cell receptor D region, at least a portion of the immune cell receptor C region, and at least a portion of the immune cell receptor J region;
c) hybridizing a first universal adaptor to the [SPLINT] adaptor hybridization site of the extended immune cell receptor V gene specific primers, wherein said universal adaptor is a double-stranded adaptor, comprising: a 5′ single-stranded overhang region which can be hybridized to the SPLINT adaptor hybridization site of said extended primers;
d) ligating the hybridized first universal adapters to the extended immune cell receptor V gene specific primers, and then removing un-ligated adapters, if present;
e) hybridizing a plurality of immune cell receptor J gene specific primers to the J region portions of the extended immune cell receptor V gene specific primers, wherein the immune cell receptor J gene specific primers comprise a 3′ J gene hybridizing region and a 5′ second universal adapter region or hybridizing a plurality of immune cell receptor C gene specific primers to the C region portions of the extended immune cell receptor V gene specific primers, wherein the immune cell receptor C gene specific primers comprise a 3′ C gene hybridizing region and a 5′ second universal adapter region; and
f) extending the hybridized immune cell receptor J gene specific primers or C gene specific primers with a polymerase, thereby forming a plurality of structurally different double-stranded products, each comprising at least a portion of the immune cell receptor V region, optionally the immune cell receptor D region, and at least a portion of the immune cell receptor J region or C region flanked by a first and second universal adapter sequence;
g) amplifying the plurality of structurally different double-stranded products with thereby enriching the plurality of structurally different target polynucleotides comprising immune gene sequences; and
h) determining nucleotide sequences of the plurality of structurally different target polynucleotides comprising the immune specific gene sequences from the products.

In some embodiments, the method further comprises
i) comparing the quantity or identity of sequences of the plurality of structurally different target polynucleotides comprising the immune gene sequences targeting the antigen (e.g., HPA-1a antigen in the case of FNAIT) from at least one sample to sequences of polynucleotides comprising the immune gene sequences targeting the antigen from a control or from a second sample obtained at an earlier time point in the woman's pregnancy.

In some embodiments, the removing of b) or the removing of d) or both comprise contacting the sample with an exonuclease.

In some embodiments, the method further comprises:
administering one or more agent or treatment to the woman or fetus to reduce the effect of the alloimmune disorder or pregnancy (for example FNAIT) on the fetus.

Also provided is a method of quantifying immune specific genes in a pregnant woman carrying a fetus encoding an antigen associated with alloimmune disorder of pregnancy (e.g., HPA-1a in the case of FNAIT), wherein the pregnant woman lacks the antigen. In some embodiments, the method comprises:
a) providing a reaction mixture comprising:
i) the plurality of structurally different target polynucleotides from the pregnant woman; and
ii) a plurality of immune cell receptor C gene specific primers, wherein the plurality of structurally distinct primers having the following regions from 5′ to 3′: [5′-Phos], [SPLINT], [BARCODE], and [FW], wherein:
[5′-Phos] comprises a 5′ phosphate;
[SPLINT] comprises an adaptor hybridization site of 2-8 nucleotides in length;
[BARCODE] comprises a barcode region of at least 6 nucleotides in length, wherein each nucleotide of the barcode region is independently selected from the group consisting of N and W; and
[FW] of each immune cell receptor C gene specific primer comprises a structurally distinct region that specifically hybridizes to an immune cell receptor C gene,
wherein the immune cell receptor C gene specific primers are hybridized to the C gene regions of the target polynucleotides;
b) extending the hybridized immune cell receptor C gene specific primers with a polymerase, and then removing un-extended immune cell receptor C gene specific primers, if present, wherein the extended immune cell receptor C gene specific primers comprise at least a portion of the immune cell receptor C region, optionally the immune cell receptor D region, at least a portion of the immune cell receptor J region, and at least a portion of the immune cell receptor V region;
c) hybridizing a first universal adaptor to the [SPLINT] adaptor hybridization site of the extended immune cell receptor C gene specific primers;
d) ligating the hybridized first universal adapters to the extended immune cell receptor C gene specific primers, and then removing un-ligated adapters, if present;
e) hybridizing a plurality of immune cell receptor V gene specific primers to the V region portions of the extended immune cell receptor C gene specific primers, wherein the immune cell receptor V gene specific primers comprise a 3′ V gene hybridizing region and a 5′ second universal adapter region; and
f) extending the hybridized immune cell receptor V gene specific primers with a polymerase, thereby forming a plurality of structurally different double-stranded products, each comprising at least a portion of the immune cell receptor V region, optionally the immune cell receptor D region, and at least a portion of the immune cell C region flanked by a first and second universal adapter sequence;
g) amplifying the plurality of second structurally different double-stranded products thereby enriching the plurality of structurally different target polynucleotides comprising an immune gene sequence; and
h) determining nucleotide sequences of the plurality of structurally different target polynucleotides comprising the immune gene sequence from the products.

In some embodiments, the method further comprises
i) comparing the quantity or identity of sequences of the plurality of structurally different target polynucleotides comprising the immune gene sequences targeting the antigen (e.g., HPA-1a antigen in the case of FNAIT) from at least one sample to sequences of polynucleotides comprising the immune gene sequences targeting the antigen from a control or from a second sample obtained at an earlier time point in the woman's pregnancy.

In some embodiments, the removing of b) or the removing or d) or both comprise contacting the sample with an exonuclease.

In some embodiments, the method further comprises:
administering one or more agent or treatment to the woman or fetus to reduce the effect of the alloimmune disorder (e.g., FNAIT) on the fetus.

In other embodiments, the method comprises
a) providing one or more sample comprising DNA from the pregnant woman;
b) contacting the sample with a plurality of immune cell receptor V gene specific primers, each primer including from 5′ to 3′: [SPLINT1], [BARCODE], and [V], wherein: [SPLINT1] is a first adaptor sequence; [BARCODE] is a unique molecular identifier barcode; and [V] is a sequence capable of hybridizing to an immune cell receptor V gene;
c) hybridizing and extending the V gene specific primers to form a plurality of first double-stranded primer extension products;
d) removing unhybridized V gene specific primers from the first double stranded primer extension products;
e) contacting the sample with a plurality of immune cell receptor J gene specific primers, each primer including from 5′ to 3′: [SPLINT2], and [J], wherein: [SPLINT2] is a second adaptor sequence; and [J] is a sequence capable of hybridizing to an immune cell receptor J gene; and further contacting the sample with a first universal primer capable of hybridizing to the first adaptor sequence;
f) hybridizing and extending the J gene specific primers and the first universal primer to form a plurality of second double-stranded primer extension products;
g) removing unhybridized J gene specific primers and first universal primer from the second double-stranded primer extension products;
h) contacting the sample with first and second universal primers capable of hybridizing to the first and second adaptor sequences;
i) amplifying the plurality of second double-stranded primer extension products thereby enriching the plurality of structurally different target polynucleotides comprising an immune gene sequence; and
j) determining nucleotide sequences of the plurality of structurally different target polynucleotides comprising the immune gene sequence from the products.

In other embodiments, the method further comprises
k) comparing the quantity or identity of sequences of the plurality of structurally different target polynucleotides comprising the immune gene sequences targeting the antigen from at least one sample to sequences of polynucleotides comprising the immune gene sequences targeting the antigen from a control or from a second sample obtained at an earlier time point in the woman's pregnancy.

In other embodiments, the d) removing or g) removing or both comprise contacting the sample with an exonuclease.

In other embodiments, the method further comprises administering one or more agent
or treatment to the woman or fetus to reduce the effect of the alloimmune disorder (e.g., FNAIT) on the fetus.

### BRIEF DESCRIPTION OF THE DRAWINGS

FIGS. 1A-C depict exemplary immunoPETE methods that do not involve ligation. FIG. 1A depicts primer extension with a primer that anneals to the V-region, wherein the primer includes a UMI barcode and a SPLINT1 sequence, which as depicted is a 3′ of the i7 adapter sequence. Following primer extension, unreacted components are digested with exonuclease or otherwise removed. FIG. 1B depicts a second primer extension step with a primer that anneals to the J region and that includes a SPLINT 2 sequence, which as depicted is a 3′ portion of the i5 sequence. Included in this second primer extension step is a first universal primer capable of annealing to the SPLINT1 sequence that is extended in a template-specific manner. FIG. 1C depicts the resulting double-stranded amplicon resulting for the two primer extension steps and subsequent amplification of the double-stranded amplicon with primers that anneal to the SPLINT1 and SPLINT2 sequences, in this case resulting in a longer amplicon having reconstituted full length i5 and i7 sequences at their ends.

### DEFINITIONS

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Spring Harbor Lab Press (Cold Spring Harbor, NY 1989). The term “a” or “an” is intended to mean “one or more.” The term “comprise,” and variations thereof such as “comprises” and “comprising,” when preceding the recitation of a step or an element, are intended to mean that the addition of further steps or elements is optional and not excluded. Any methods, devices and materials similar or equivalent to those described herein can be used in the practice of this invention. The following definitions are provided to facilitate understanding of certain terms used frequently herein and are not meant to limit the scope of the present disclosure.

As used herein, the term “substantially all” in reference to removing substantially all of a component of a reaction mixture means removing at least 90%, 95%, 99%, or more of a component.

As used herein, the term “immune cell receptor” refers to a T cell receptor (TCR), or a B cell receptor (BCR) (i.e., antibody). The BCR can be in a membrane bound form or a secreted form.

“T cell receptor” or “TCR” refers to the antigen recognition complex of a T cell. The TCR is composed of two different protein chains (e.g., alpha and beta or gamma and delta). Each chain is composed of two extracellular domains containing a variable region (V), a joining region (J), and a constant region (C). The variable region contains hypervariable complementarity determining regions (CDRs). Beta and delta TCR chains further contain a diversity region (D) between the V and J regions. Further TCR diversity is generated by VJ (for alpha and gamma chains) and VDJ (for beta and delta chains) recombination. The terms also refer to various recombinant and heterologous forms, including soluble TCRs expressed from a heterologous system.

The B cell receptor or “BCR” refers to the secreted or membrane bound antigen recognition complex of a B cell. The BCR is composed of two different protein chains (e.g., heavy and light). Each chain contains a variable region (V), a joining region (J), and a constant region (C). The variable region contains hypervariable complementarity determining regions (CDRs). Heavy chains can further contain a diversity region (D) between the V and J regions. Further BCR diversity is generated by VJ (for light chains) and VDJ (for heavy chains) recombination as well as somatic hypermutation of recombined chains. The terms also refer to various recombinant and heterologous forms.

As used herein, the term “barcode” refers to a nucleic acid sequence that can be detected and identified. Barcodes can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides long. Barcodes can employ error-correcting codes such that one or more errors in synthesis, replication, and/or sequencing can be corrected to identify the barcode sequence. Examples of error correcting codes and their use in barcodes and barcode identification and/or sequencing include, but are not limited to, those described in U.S. 2010/0,323,348; and U.S. Pat. No. 8,715,967. In some cases, the barcodes are designed to have a minimum number of distinct nucleotides with respect to all other barcodes of a population. The minimum number can be 1, 2, 3, 4, 5, 6, 7, 8, 9, 10, 11, 12, 13, 14, 15, or more. Thus, for example, a population of barcodes having a minimum number of at least five distinct nucleotides will differ at least five nucleotide positions from all other barcodes in the population.

As used herein, the term “multiplex identifier,” “MID,” and the like, refers to a barcode that identifies a source or sample. As such, all or substantially all, MID barcoded polynucleotides from a single source or sample will share an MID of the same sequence; while all, or substantially all (e.g., at least 90% or 99%), MID barcoded polynucleotides from different sources or samples will have a different MID barcode sequence. Polynucleotides from different sources or samples and having different MIDs can then be mixed and sequenced in parallel while maintaining source/sample information. Thus sequence reads can be assigned to individual samples.

As used herein, the term “universal identifier,” “universal molecular identifier,” “unique molecular identifier,” “UID,” and the like, refers to a barcode that identifies a polynucleotide to which it is attached. In some cases, all, or substantially all (e.g., at least 90% or 99%), UID barcodes in a mixture of UID barcoded polynucleotides are unique. In other cases, the universal identifier sequence is rare but not unique and it, in combination with other information about the sequence to which it is attached, such as library barcodes, target (e.g., CDR3) sequence information or additional sequence information can be used to uniquely identify a sequence read.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologues, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

“Polymerase” refers to an enzyme that performs template-directed synthesis of polynucleotides. A DNA polymerase can add free nucleotides only to the 3′ end of the newly forming strand. This results in elongation of the newly forming strand in a 5′-3′ direction. No known DNA polymerase is able to begin a new chain (de novo). DNA polymerase can add a nucleotide only on to a pre-existing 3′—OH group, and, therefore, needs a primer at which it can add the first nucleotide. Non-limiting examples of polymerases include prokaryotic DNA polymerases (e.g. Pol I, Pol II, Pol III, Pol IV and Pol V), eukaryotic DNA polymerase, telomerase, reverse transcriptase and RNA polymerase. Reverse transcriptase is an RNA-dependent DNA polymerase that synthesizes DNA from an RNA template. The reverse transcriptase family contain both DNA polymerase functionality and RNase H functionality, which degrades RNA base-paired to DNA. RNA polymerase is an enzyme that synthesizes RNA using DNA as a template during the process of gene transcription. RNA polymerase polymerizes ribonucleotides at the 3′-end of an RNA transcript.

In some embodiments, a polymerase from the following may be used in a polymerase-mediated primer extension, end-modification (e.g., terminal transferase, degradation, or polishing), or amplification reaction: archaea (e.g., Thermococcus litoralis (Vent, GenBank: AAA72101), Pyrococcus furiosus (Pfu, GenBank: D12983, BAA02362), Pyrococcus woesii, Pyrococcus GB-D (Deep Vent, GenBank: AAA67131), Thermococcus kodakaraensis KODI (KOD, GenBank: BD175553, BAA06142; Thermococcus sp. strain KOD (Pfx, GenBank: AAE68738)), Thermococcus gorgonarius (Tgo, Pdb: 4699806), Sulfolobus solataricus (GenBank: NC002754, P26811), Aeropyrum pernix (GenBank: BAA81109), Archaeglobus fulgidus (GenBank: 029753), Pyrobaculum aerophilum (GenBank: AAL63952), Pyrodictium occultum (GenBank: BAA07579, BAA07580), Thermococcus 9 degree Nm (GenBank: AAA88769, Q56366), Thermococcus fumicolans (GenBank: CAA93738, P74918), Thermococcus hydrothermalis (GenBank: CAC18555), Thermococcus sp. GE8 (GenBank: CAC12850), Thermococcus sp. JDF-3 (GenBank: AX135456; WO0132887), Thermococcus sp. TY (GenBank: CAA73475), Pyrococcus abyssi (GenBank: P77916), Pyrococcus glycovorans (GenBank: CAC12849), Pyrococcus horikoshii (GenBank: NP 143776), Pyrococcus sp. GE23 (GenBank: CAA90887), Pyrococcus sp. ST700 (GenBank: CAC 12847), Thermococcus pacificus (GenBank: AX411312.1), Thermococcus zilligii (GenBank: DQ3366890), Thermococcus aggregans, Thermococcus barossii, Thermococcus celer (GenBank: DD259850.1), Thermococcus profundus (GenBank: E14137), Thermococcus siculi (GenBank: DD259857.1), Thermococcus thioreducens, Thermococcus onnurineus NA1, Sulfolobus acidocaldarium, Sulfolobus tokodaii, Pyrobaculum calidifontis, Pyrobaculum islandicum (GenBank: AAF27815), Methanococcus jannaschii (GenBank: Q58295), Desulforococcus species TOK, Desulforococcus, Pyrolobus, Pyrodictium, Staphylothermus, Vulcanisaetta, Methanococcus (GenBank: P52025) and other archaeal B polymerases, such as GenBank AAC62712, P956901, BAAA07579)), thermophilic bacteria Thermus species (e.g., flavus, ruber, thermophilus, lacteus, rubens, aquaticus), Bacillus stearothermophilus, Thermotoga maritima, Methanothermus fervidus, KOD polymerase, TNA1 polymerase, Thermococcus sp. 9 degrees N-7, T4, T7, phi29, Pyrococcus furiosus, P. abyssi, T. gorgonarius, T. litoralis, T. zilligii, T. sp. GT, P. sp. GB-D, KOD, Pfu, T. gorgonarius, T. zilligii, T. litoralis and Thermococcus sp. 9N-7 polymerases.

In some embodiments, the polymerase can include a RNA polymerase from an eukaryote such as RNA polymerase I, RNA polymerase II, RNA polymerase III, RNA polymerase IV or

RNA polymerase V. The polymerase can include a bacterial RNA polymerase as well as phage and viral RNA polymerases. In some aspects, the RNA polymerase can include a T7 RNA polymerase, T3 RNA polymerase, K11 RNA polymerase, K1F RNA polymerase, N4 RNA polymerase or SP6 RNA polymerase. In some embodiments, the RNA polymerase (e.g., E. coli RNA polymerase) can include a wild-type, mutant or artificially engineered RNA polymerase. As used herein, “an artificially engineered RNA polymerase” consists, or comprises, of at least one amino acid substitution, deletion or insertion with respect to the wild-type or naturally occurring RNA polymerase from which the artificially engineered RNA polymerase is obtained or derived.

In some embodiments, the polymerase can include a reverse transcriptase (RT). RT is an RNA-dependent DNA polymerase that synthesizes double-stranded DNA (cDNA) from a single-stranded RNA template. The RT family contain both DNA polymerase functionality and RNase H functionality, which degrades RNA base-paired to DNA. RT's are predominately associated with retroviruses although non-retroviruses also use RT (e.g., Hepatitis B virus). In some aspects, the polymerase can include a RT from Human Immunodeficiency Virus (e.g., HIV-1), Moloney Murine Leukemia Virus (e.g., M-MLV), Human T-Lymphotrophic Virus (e.g., HTLV), Avian Myeloblastosis Virus (e.g., AMV), Rous Sarcoma Virus (e.g., RSV), SuperScript RT (e.g., SuperScript IV), or Telomerase RT (e.g., TERT). In some embodiments, the RT can include a recombinant RT (e.g., an RT having one or more amino acid substitutions, deletions or additions as compared to the corresponding wild-type or naturally occurring RT). In some aspects, the RT includes transcription of RNA fragments of various sizes (e.g., 1 kb, 5 kb, 10 kb, 15 kb, 20 kb, or more).

The term “thermostable polymerase,” refers to an enzyme that is stable to heat, is heat resistant, and retains sufficient activity to effect subsequent polynucleotide extension reactions and does not become irreversibly denatured (inactivated) when subjected to the elevated temperatures for the time necessary to effect denaturation of double-stranded nucleic acids. The heating conditions necessary for nucleic acid denaturation are well known in the art and are exemplified in, e.g., U.S. Pat. Nos. 4,683,202, 4,683,195, and 4,965,188, which are incorporated herein by reference. As used herein, a thermostable polymerase is suitable for use in a temperature cycling reaction such as the polymerase chain reaction (“PCR”), a primer extension reaction, or an end-modification (e.g., terminal transferase, degradation, or polishing) reaction. Irreversible denaturation for purposes herein refers to permanent and complete loss of enzymatic activity. For a thermostable polymerase, enzymatic activity refers to the catalysis of the combination of the nucleotides in the proper manner to form polynucleotide extension products that are complementary to a template nucleic acid strand. Thermostable DNA polymerases from thermophilic bacteria include, e.g., DNA polymerases from Thermotoga maritima, Thermus aquaticus, Thermus thermophilus, Thermus flavus, Thermus filiformis, Thermus species sps17, Thermus species Z05, Thermus caldophilus, Bacillus caldotenax, Thermotoga neopolitana, Thermosipho africanus, and other thermostable DNA polymerases disclosed above.

In some cases, the nucleic acid (e.g., DNA or RNA) polymerase may be a modified naturally occurring Type A polymerase. A further embodiment of the invention generally relates to a method wherein a modified Type A polymerase, e.g., in a primer extension, end-modification (e.g., terminal transferase, degradation, or polishing), or amplification reaction, may be selected from any species of the genus Meiothermus, Thermotoga, or Thermomicrobium. Another embodiment of the invention generally pertains to a method wherein the polymerase, e.g., in a primer extension, end-modification (e.g., terminal transferase, degradation or polishing), or amplification reaction, may be isolated from any of Thermus aquaticus (Taq), Thermus thermophilus, Thermus caldophilus, or Thermus filiformis. A further embodiment of the invention generally encompasses a method wherein the modified Type A polymerase, e.g., in a primer extension, end-modification (e.g., terminal transferase, degradation, or polishing), or amplification reaction, may be isolated from Bacillus stearothermophilus, Sphaerobacter thermophilus, Dictoglomus thermophilum, or Escherichia coli. In another embodiment, the invention generally relates to a method wherein the modified Type A polymerase, e.g., in a primer extension, end-modification (e.g., terminal transferase, degradation, or polishing), or amplification reaction, may be a mutant Taq-E507K polymerase. Another embodiment of the invention generally pertains to a method wherein a thermostable polymerase may be used to effect amplification of the target nucleic acid.

As used herein the term “primer” refers to an oligonucleotide that binds to a specific region of a single stranded template nucleic acid molecule and initiates nucleic acid synthesis via a polymerase-mediated enzymatic reaction, extending from the 3′ end of the primer and complementary to the sequence of the template molecule. PCR amplification primers can be referred to as ‘forward’ and ‘reverse’ primers, one of which is complementary to a nucleic acid strand and the other of which is complementary to the complement of that strand. Typically, a primer comprises fewer than about 100 nucleotides and preferably comprises fewer than about 30 nucleotides. Exemplary primers range from about 5 to about 25 nucleotides. Primers can comprise, for example, RNA and/or DNA bases, as well as non-naturally-occurring bases. The directionality of the newly forming strand (the daughter strand) is opposite to the direction in which DNA polymerase moves along the template strand.

As used herein, the term “universal primer” and “universal primers” refers to a primer that can hybridize to and support amplification of target polynucleotides having a shared complementary universal primer binding site. Similar, the term “universal primer pair” refers to a forward and reverse primer pair that can hybridize to and support PCR amplification of target polynucleotides having shared complementary forward and reverse universal primer binding sites. Such universal primer(s) and universal primer binding site(s) can allow single or double-primer mediated universal amplification (e.g., universal PCR) of target polynucleotide regions of interest.

As used herein the term “sample” refers to any biological sample that comprises nucleic acid molecules, typically comprising DNA and/or RNA. Samples may be tissues, cells or extracts thereof, or may be purified samples of nucleic acid molecules. Use of the term “sample” does not necessarily imply the presence of target sequence within nucleic acid molecules present in the sample. In some cases, the “sample” comprises immune cells (e.g., B cells and/or T cells), or a fraction thereof (e.g., a fraction enriched in genomic DNA, total RNA, or mRNA). In some embodiments, a sample can comprise a FACS sorted population of cells (such as human T cells) or a fixed formalin paraffin embedded (FFPE) tissue sample.

As used herein, the phrase “stringent hybridization conditions” refers to conditions under which a primer/probe will hybridize to its target, typically in a complex mixture of nucleic acids, but not to other nucleic acid sequences present in the complex mixture. Stringent conditions are sequence-dependent and will be different under different circumstances. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, high stringent conditions are selected to be about 5-10° C. lower than the thermal melting point (Tₘ) for the specific sequence at a defined ionic strength pH. Low stringency conditions are generally selected to be about 15-30° C. below the Tₘ. The Tₘ is the temperature (under defined ionic strength, pH, and nucleic concentration) at which 50% of the primers/probes complementary to the target hybridize to the target sequence at equilibrium. Stringent conditions include those in which the salt concentration is less than about 1.0 M sodium ion, typically about 0.01 to 1.0 M sodium ion concentration (or other salts) at pH 7.0 to 8.3 and the temperature is at least about 30° C. for short primers/probes (e.g., 10 to 50 nucleotides) and at least about 60° C. for long primers/probes (e.g., greater than 50 nucleotides). Stringent conditions may also be achieved with the addition of destabilizing agents such as formamide.

### DETAILED DESCRIPTION OF THE INVENTION

Methods of using immune repertoire profiling for identifying pregnant women carrying a fetus having an enhanced likelihood of developing an alloimmune disorder of pregnancy (which can include but is not limited to, FNAIT, HDFN or alloimmune neutropenia), to monitor for the development of the alloimmune disorder in at-risk pregnancies, or to monitor the severity of known cases of alloimmune disorder are provided. Women identified to be negative for a human platelet antigen associated with the alloimmune disorder of pregnancy, but carrying a fetus that expresses the antigen, can be further assessed via immune profiling technologies, for example, to detect immune repertoire signatures indicative of development of the alloimmune disorder of pregnancy or worsening of a known alloimmune disorder of pregnancy.

As noted above, the described methods are useful for assessing the likelihood that an alloimmune disorder of pregnancy (for example but not limited to FNAIT, HDFN or alloimmune neutropenia) will occur in a fetus (i.e., due to an immune response from the pregnant woman), monitoring for development of the alloimmune disorder of pregnancy in an at-risk pregnancy or monitoring for worsening of a known alloimmune disorder of pregnancy (i.e., due to an upward trend or clonal expansion in the pregnant woman's immune response). Thus, in some embodiments, initially the pregnant woman and fetus are screened for a discordance in one or more alloantigen(s) associated with the alloimmune disorder of pregnancy, wherein if the pregnant woman is negative for carrying the allele for the relevant antigen and the fetus carries the allele for the relevant antigen, then there is a risk of occurrence of the alloimmune disorder of pregnancy. The methods described herein do not require an initial screen for alloantigens in the pregnant woman or fetus but pre-screening for alloantigens and limiting immune profiling to discordant pregnancies will greatly reduce the number of assays required to identify the alloimmune disorder of pregnancy in a population of pregnant women.

Alloimmune disorders of pregnancy involve transplacentally-transferred maternal (e.g., IgG) antibodies binding to fetal antigens foreign to the mother, inherited by the fetus from the father. Exemplary alloimmune disorders of pregnancy include but are not limited to FNAIT, hemolytic disease of the fetus and newborn (HDFN) and alloimmune neutropenia. FNAIT involves targeting by the maternal immune system of fetal platelet antigens. HPA-1a is the most frequently implicated human platelet antigen for causing FNAIT in Caucasians. See, e.g., Tiller, et al., Int J Womens Health. 2017; 9: 223-234. Other alloantigens implicated in FNAIT include, for example, HPA-2, -3, -4, -5, and -15. See, e.g., Zdravic D, Yougbare I, Vadasz B, et al. Semin Fetal Neonatal Med. 2016; 21(1):19-27 for a listing of additional FNAIT alloantigens. Three fetal antigens that can be targeted in HDFN by the maternal immune system include: Rh (rhesus), minor red cell antigens (i.e., Kell, Duffy, Kidd antigens) and ABO. Fetal/neonatal alloimmune neutropenia involves maternal sensitization to paternal-derived antigens present on the fetal neutrophils. See, e.g., Porcelijn et al, Transfus Med Hemother. 2018 October; 45(5): 311-316.

The methods described herein can involve generating an immune profile from one or more sample from a pregnant woman that contains immune-specific gene nucleic acids (e.g., DNA or mRNA). In one embodiment, complementary DNA (cDNA) can be prepared from RNA or mRNA for use in the primer extension target enrichment (PETE) methods described herein. Flanking primer extension target enrichment methods for immune repertoire profiling workflows is termed “immunoPETE.” In one aspect, cDNA is prepared from total RNA or mRNA isolated and/or purified from a cell, cell lysate, sample or tissue from the woman. In one embodiment, RNA can include total RNA obtained using RNA isolation and/or extraction methods known in the art (e.g., Molecular Cloning: A Laboratory Manual, 4th Edition, Vol. 1, Chapter 6 (2012) incorporated herein by reference for all purposes; RNeasy Mini Kit (Catalog Number: 74104), Qiagen, Germantown, MD). In another embodiment, cDNA can be synthesized from total RNA or mRNA transcripts using commercially available kits (for example, SuperScript III™ Reverse Transcriptase (Catalog Number: 18080093) or SuperScript® VILO™ cDNA Synthesis Kit (Catalog Number: 11754050), ThermoFisher Scientific, Waltham, MA). In another embodiment, cDNA can be prepared from total RNA or mRNA transcripts obtained from whole blood, peripheral blood mononuclear cell (PBMC), sorted lymphocytes, lymphocyte culture, fresh or fresh-frozen tumor tissue or formalin-fixed paraffin embedded (FFPE) tissue (for example, RNeasy FFPE kit, Qiagen, Germantown, MD). In one aspect, cDNA synthesis can be initiated at or near the 3′ termini of the mRNA transcript and terminates at or near the 5′ end of the mRNA so as to generate “full-length” cDNA molecules.

ImmunoPETE methods such as described in, e.g., U.S. Patent Application Publication Nos. US2021/0254157, US2021/0172015, US2018/0087108 and US2020/0385707 can be used to generate an immune profile. In some embodiments, ImmunoPETE methods comprise forming a reaction mixture and subsequently generating an immune profile. In some embodiments, one forms a composition comprising: i) a plurality of structurally distinct immune cell receptor V gene specific primers, wherein the plurality comprises at least 10 structurally distinct primers having the following regions from 5′ to 3′: [5′-Phos], [SPLINT], [BARCODE], and [FW], wherein: [5′-Phos] comprises a 5′ phosphate; [SPLINT] comprises an adaptor hybridization site of 2-8 nucleotides in length; [BARCODE] comprises a barcode region of at least 6 nucleotides in length, wherein each nucleotide of the barcode region is independently selected from the group consisting of N and W; and [FW] of each immune cell receptor V gene specific primer comprises a structurally distinct region that specifically hybridizes to a framework 1, framework 2, or framework 3 region of an immune cell receptor V gene from the sample. In some embodiments, [BARCODE] comprises a barcode region of from 6 to 16 nucleotides in length. In some embodiments, the [FW] of each immune cell receptor V gene specific primer specifically hybridizes to a framework 1, framework 2, or framework 3 region of a T cell receptor V gene from the sample. In some embodiments, the [FW] of each immune cell receptor V gene specific primer specifically hybridizes to a framework 1, framework 2, or framework 3 region of a B cell receptor V gene from the sample. In some embodiments, the plurality comprises at least 10 of the primers set forth in SEQ ID Nos:1-121. In some embodiments, the [SPLINT] consists of 6 consecutive nucleotides, preferably of the sequence CGA TCT. In some embodiments, [BARCODE] consists of thirteen consecutive nucleotides selected from the group consisting of N and W, preferably of the sequence WNN NNN WNN NNN W. In some embodiments, the composition comprises at least 50, preferably all of the primers set forth in SEQ ID NOs:1-121.

In some embodiments, a reaction mixture is formed comprising: i) a plurality of structurally different target polynucleotides from the sample, wherein individual target polynucleotides of the plurality each comprise immune cell receptor V gene regions, optionally D gene regions, optionally C gene regions, and J gene regions; and ii) a plurality of immune cell receptor V gene specific primers according to any one of the preceding aspects or embodiments. In some embodiments, the plurality of immune cell receptor V gene specific primers are each hybridized to one of the plurality of structurally different target polynucleotides.

In some embodiments, a portion of the individual target polynucleotides of the plurality each comprise immune cell receptor D gene regions and a portion of the individual target polynucleotides of the plurality do not comprise immune cell receptor D gene regions. In some embodiments, the individual target polynucleotides of the plurality each comprise immune cell receptor D gene regions. In some embodiments, a portion of the individual target polynucleotides of the plurality each comprise immune cell receptor C gene regions and a portion of the individual target polynucleotides of the plurality do not comprise immune cell receptor C gene regions. In some embodiments, the individual target polynucleotides of the plurality each comprise immune cell receptor C gene regions. In some embodiments, the reaction mixture comprises a plurality of immune cell receptor C gene specific (C-segment) primers. In some embodiments, the plurality of immune cell receptor C gene specific primers are hybridized to one of the structurally different target polynucleotides.

In some embodiments, a reaction mixture is formed comprising: i) a plurality of immune cell receptor specific first primer extension products, wherein the individual first primer extension products each comprise the following from regions from 5′ to 3′: a sequencer-specific adapter sequence, optionally a multiplex identifier (MID) barcode, a unique molecular identifier (UID) barcode, at least a portion of an immune cell receptor framework 3 region, an immune cell receptor CDR3 region, an optional immune cell receptor diversity (D) region, an optional immune cell receptor constant (C) region, and at least a portion of an immune cell receptor J region from the sample; and ii) a plurality of J-gene specific primers, wherein each of the plurality of J-gene specific primers is hybridized to the immune cell receptor J region of one of the individual first primer extension products or a plurality of C gene specific primers, wherein each of the plurality of C gene specific primers is hybridized to the immune cell receptor C region of one of the individual first primer extension products.

In some embodiments, the reaction mixture further comprises a DNA polymerase. In some embodiments, the individual first primer extension products each comprise the multiplex identifier (MID) barcode. In some embodiments, a portion of the individual first primer extension products comprise the immune cell receptor D region and a portion of the first primer extension products do not comprise the immune cell receptor D region. In some embodiments, the individual target polynucleotides from the sample comprise the immune cell receptor D region. In some embodiments, the plurality of J-gene specific primers comprise at least 10 of the primers set forth in SEQ ID Nos:122-204. In some embodiments, the plurality of J-gene specific primers comprise at least 50, preferably all of the primers set forth in SEQ ID Nos: 122-204. In some embodiments, the individual target polynucleotides each comprise the immune cell receptor C region. In some embodiments, the plurality of C gene specific primers comprise at least two of the primers set forth in SEQ ID NOs:205-213. In some embodiments, the plurality of C gene specific primers comprise at least five, preferably all, of the primers set forth in SEQ ID NOs:205-213.

In some embodiments, a reaction mixture is formed comprising: i) a plurality of first primer extension products, the individual first primer extension products each comprising the following from 5′ to 3′: a) a 5′ phosphate; b) a SPLINT region comprising an adaptor hybridization site of 2-8 nucleotides in length; c) a unique molecular identifier (UID) barcode; d) at least a portion of an immune cell receptor framework 3 region from the sample; e) an immune cell receptor CDR3 region; f) an optional immune cell receptor diversity region; g) at least a portion of an immune cell receptor J region; and h) an optional immune cell receptor constant region; and i) a plurality of double-stranded splint adapters, each comprising: a) a 5′ single-stranded overhang region hybridized to the SPLINT region of an individual first primer extension product; b) optionally a multiplex identifier (MID) barcode; and c) a sequencer-specific universal primer sequence.

In some embodiments, the reaction mixture further comprises ligase. In some embodiments, the double-stranded splint adapters each comprise a multiplex identifier (MID) barcode.

In some embodiments, the ImmunoPETE methods comprise a method for enriching from a sample a plurality of structurally different target polynucleotides from a sample, wherein individual target polynucleotides of the plurality comprise immune cell receptor V, J, and optionally C and/or D gene regions, the method comprising: a) providing a reaction mixture according to any one of the preceding aspects or embodiments or as otherwise described herein or in U.S. Patent Application Publication Nos. US2021/0254157, US2021/0172015, US2018/0087108 or US2020/0385707 that provide a reaction mixture, wherein the immune cell receptor V gene specific primers are hybridized to the V gene regions of the target polynucleotides; b) extending the hybridized immune cell receptor V gene specific primers with a polymerase, and then removing un-extended immune cell receptor V gene specific primers, if present, wherein the extended immune cell receptor V gene specific primers comprise at least a portion of the immune cell receptor V region, optionally the immune cell receptor D region, and at least a portion of the immune cell receptor J region; c) hybridizing a first universal adaptor to the [SPLINT] adaptor hybridization site of the extended immune cell receptor V gene specific primers; d) ligating the hybridized first universal adapters to the extended immune cell receptor V gene specific primers, and then removing un-ligated adapters, if present; e) hybridizing a plurality of immune cell receptor J gene specific primers to the J region portions of the extended immune cell receptor V gene specific primers, wherein the immune cell receptor J gene specific primers comprise a 3′ J gene hybridizing region and a 5′ second universal adapter region; and f) extending the hybridized immune cell receptor J gene specific primers with a polymerase, thereby forming a plurality of structurally different double-stranded products, each comprising at least a portion of the immune cell receptor V region, optionally the immune cell receptor D region, and at least a portion of the immune cell receptor J region flanked by a first and second universal adapter sequence.

In some embodiments, e) and f) are repeated 2 to 15 times by heating to denature double-stranded products, cooling to hybridize un-extended immune cell receptor J gene specific primers to the J region portions of the extended immune cell receptor V gene specific primers, and extending hybridized primers. In some embodiments, the removing un-extended immune cell receptor V gene specific primers comprises digesting single-stranded DNA exonuclease digestion. In some embodiments, the method further comprises amplifying double-stranded products comprising first and second universal adapters by universal PCR. In some embodiments, the structurally different target polynucleotides are cDNA. In some embodiments, prior to step a), a cDNA synthesis step is included to prepare cDNA from total RNA or mRNA. In some embodiments, step e) is modified such that the plurality of immune cell receptor J gene specific primers are substituted for a plurality of immune cell receptor C gene specific primers, thereby forming a plurality of structurally different double-stranded products, each comprising at least a portion of the immune cell receptor V region and at least a portion of the immune cell receptor C region.

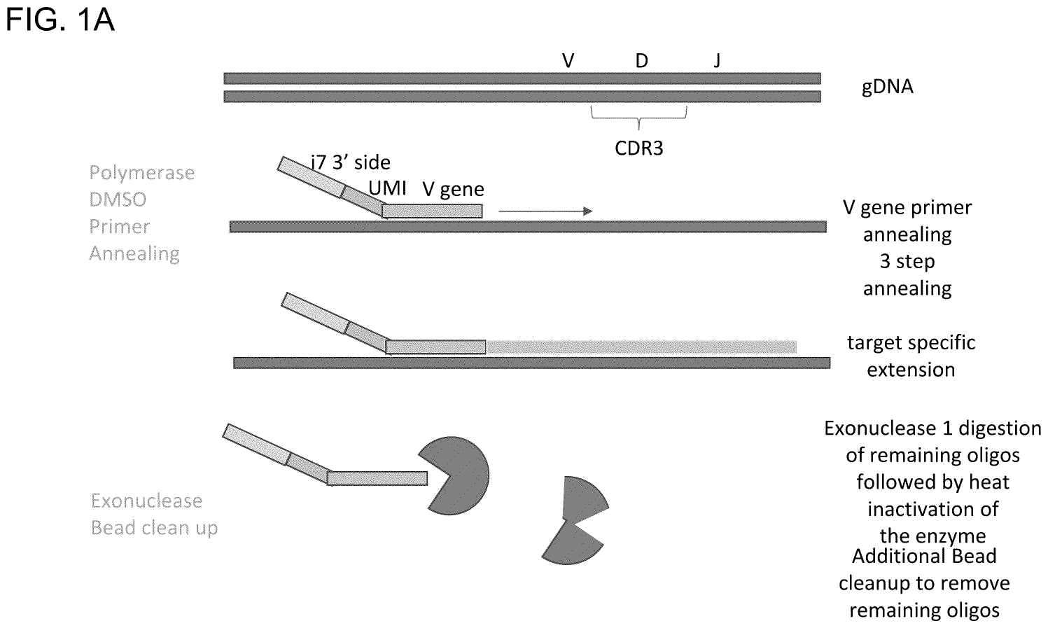

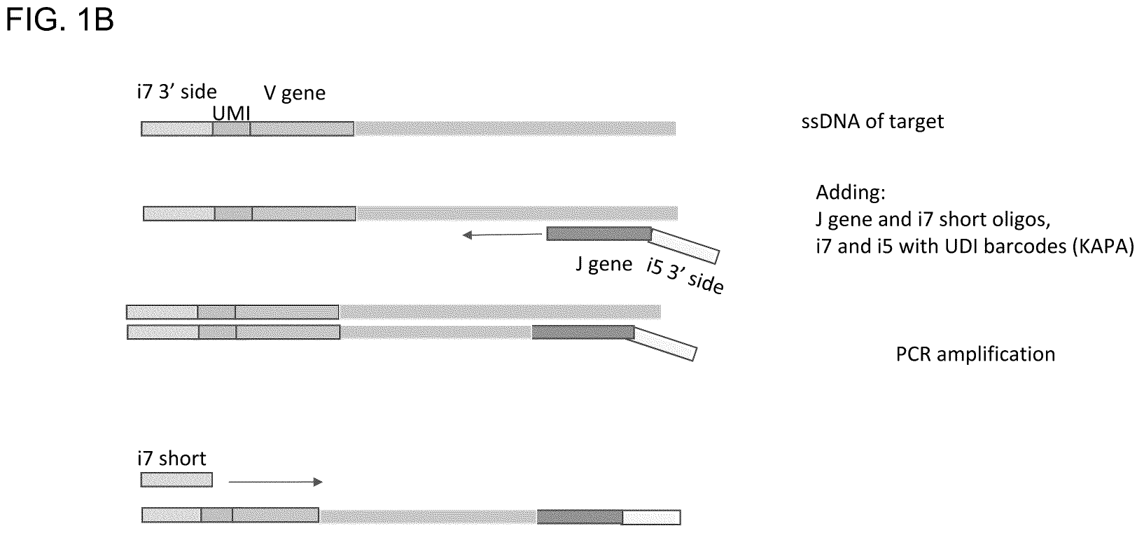

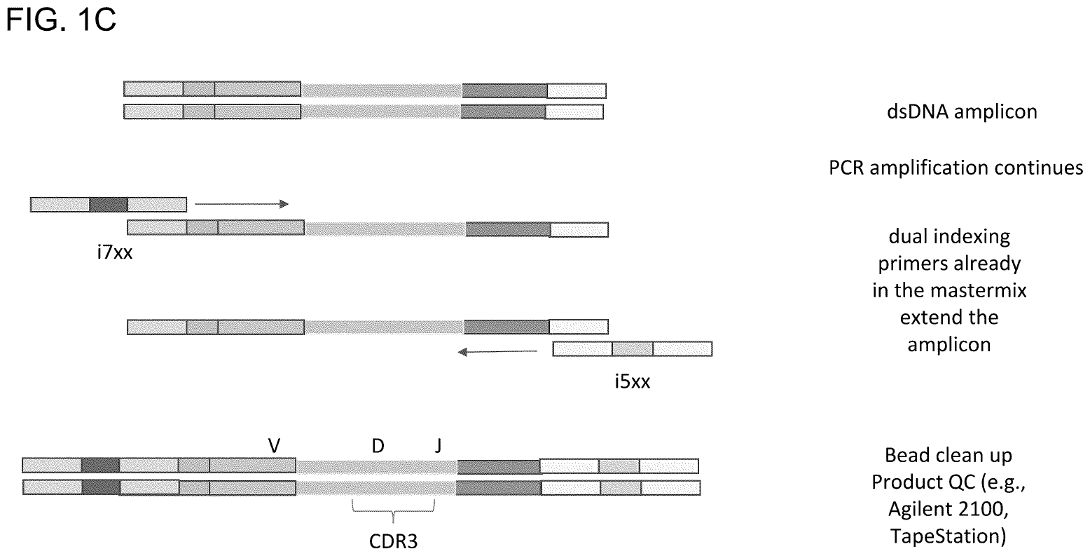

In some embodiments, the ImmunoPETE methods comprise a method for enriching a sample for a plurality of structurally different target polynucleotides comprising an immune gene sequence the method comprising: a) contacting a sample with a plurality of immune cell receptor V gene specific primers, each primer including from 5′ to 3′: [SPLINT1], [BARCODE], and [V], wherein: [SPLINT1] is a first adaptor sequence, which can be any length, for example 2-30 nucleotides; [BARCODE] is a unique molecular identifier barcode; and [V] is a sequence capable of hybridizing to an immune cell receptor V gene; b) hybridizing and extending the V gene specific primers to form a plurality of first double-stranded primer extension products; c) contacting the sample with an exonuclease to remove unhybridized V gene specific primers from the first double stranded primer extension products; d) contacting the sample with a plurality of immune cell receptor J gene specific primers, each primer including from 5′ to 3′: [SPLINT2], and [J], wherein: [SPLINT2] is a second adaptor sequence, which can be any length, for example 2-30 nucleotides; and [J] is a sequence capable of hybridizing to an immune cell receptor J gene; and further contacting the sample with a first universal primer capable of hybridizing to the first adaptor sequence; e) hybridizing and extending the J gene specific primers and the first universal primer to form a plurality of second double-stranded primer extension products; f) contacting the sample with an exonuclease to remove unhybridized J gene specific primers and first universal primer from the second double-stranded primer extension products; g) contacting the sample with first and second universal primers capable of hybridizing to the first and second adaptor sequences; h) amplifying the plurality of second double-stranded primer extension products thereby enriching the plurality of structurally different target polynucleotides comprising an immune gene sequence. In some embodiments, the immune genes comprise one or more of T-cell receptor alpha (TCRA), T-cell receptor beta (TCRB), T-cell receptor gamma (TCRG), T-cell receptor delta (TCRD), Immunoglobulin heavy chain (IGH) and Immunoglobulin light chain lambda or kappa (IGL and IGK). In some embodiments, the plurality of V gene specific primers and the plurality of J gene specific primers include primers from Table 2. In some embodiments, SPLINT1 and/or SPLINT2 sequences comprise a 3′ portion, but not all of, a universal adapter sequence, wherein the remaining portion of the universal adapter sequence is supplied by the first and second universal primers. For example, the i5 and i7 sequences are universal sequences used in Illumina-based sequencing and SPLINT 1 can comprise a portion of the i5 or i7 sequence and SPLINT 2 can include a portion of the other of i5 and i7. See, for example, FIG. 1A-C.

In some embodiments, the hybridizing in steps b) and/or e) comprises one or more cycles of a step-wise temperature drop of two or more steps, for example, 20 cycles of temperature change from 60° C. to 57.5° C. and to 55° C. In some embodiments, hybridizing and extending in step e) comprises two or more cycles of duplex denaturation, primer annealing and primer extension, for example hybridizing and extending in step e) comprises 10 cycles temperature change from >90° C., to 60° C. to 57.5° C., to 55° C. and to 72° C.

In some embodiments, the first and second universal primers in step g) comprise additional 5′ sequences not present in the first and second adaptors but the first universal primer in step d) does not comprise additional 5′ sequences not present in the first adaptor.

In some embodiments the exonuclease in steps c) and/or f) is thermolabile, e.g., a thermolabile Exonuclease I.

In some embodiments, extending in steps b) and/or e) is with a high-fidelity DNA polymerase.

In some embodiments, the method comprises a purification step after steps c) and/or h) but not between steps f) and g). In some embodiments, the first and second universal primers comprise a modification preventing digestion of the primers with the exonuclease, e.g., a phosphorothioate (PS) bond, a 2′-O-methyl (2′OMe), a 2′-fluoride and Inverted ddT. In some embodiments, the V-gene specific primers, the J-gene specific primers and the first universal primer in step d) do not comprise a modification preventing digestion of the primers with the exonuclease.

In some embodiments, the method further comprises sequencing the plurality of structurally different target polynucleotides comprising an immune gene sequence.

The use of a large number of first or second primers, a high concentration of first or second primers, or a combination thereof, can provide improved enrichment for, e.g., high-throughput sequencing sample workflows in which a large number of different polynucleotide sequences are targeted and flanking hybridization sequences for the target sequences are known. Such high-throughput sequencing sample workflows include, but are not limited to, immune repertoire profiling workflows in which B cell receptor (BCR) or T cell receptor (TCR) sequences are enriched from a sample, sequenced, and analyzed.

Hybridization conditions including those exemplified herein are readily determinable by one of ordinary skill in the art, and can include calculating primer/probe length, salt concentration, and preferential temperature to limit non-specific hybridization. In general, longer primers/probes require higher temperatures for correct annealing, while shorter primer/probes require lower temperatures. Hybridization generally depends on the ability of denatured DNA to anneal complementary nucleic acid sequences (e.g., primer/probes) present in an environment (e.g., a reaction mixture) below their melting temperature. The higher the degree of homology between a primer/probe and denatured DNA, the higher the annealing temperature can be while minimizing non-specific hybridization. Accordingly, higher relative annealing temperatures tend to make the reaction conditions more “stringent”, while lower annealing temperatures make the reaction conditions less so. Additional details and explanations of hybridization stringency can be found for example in Ausubel et al., (Current Protocols in Molecular Biology, Wiley Interscience Publishers, (1995)).

In some embodiments, the methods described herein involve a primer extension targeted gene enrichment assay designed to specifically enrich and amplify human T-cell receptor (TCR) loci and B-cell receptor (BCR) loci from genomic DNA, and result in unbiased and quantitative TCR and BCR repertoire information upon next-generation sequencing analysis. In some embodiments, the methods described herein involve a primer extension target enrichment assay optimized for the human TCR-beta locus (TRB) as well as BCR-heavy chain locus (IGH). In some embodiments, the methods described herein involve a primer extension target enrichment assay optimized for Illumina MiSeq or NextSeq next-generation sequencing platforms.

In one aspect, the methods provide first or second primers (e.g., SEQ ID NOs:1-121, 122-204, 205-213, etc.) that selectively hybridize to certain target polynucleotides. The phrase “selectively (or specifically) hybridizes to” refers to the binding, duplexing, or hybridizing of a molecule (e.g., a target polynucleotide) to a particular nucleotide sequence (e.g., a primer or probe comprising SEQ ID NOS:1-213) under stringent hybridization conditions when the target polynucleotide is present in a reaction mixture (e.g., total RNA, mRNA, cDNA or gDNA).

In one embodiment, the first or second primers described herein are complementary or substantially complementary (i.e., at least 70%, 75%, 80%, 85%, 90%, 95% or 99% complementary) to the target polynucleotides. In another embodiment, the first or second primers are complementary or substantially complementary (i.e., at least 70%, 75%, 80%, 85%, 90%, 95% or 99% complementary across at least 5, at least 10, at least 15, at least 20 or more nucleotides) to the target polynucleotides. In another embodiment, the first or second primers are complementary across their full-length to the target polynucleotides.

In another embodiment, the first or second primers are complementary or substantially complementary (i.e., at least 70%, 75%, 80%, 85%, 90%, 95% or 99% complementary) to a framework 1, framework 2, or framework 3 region of an immune cell receptor V gene. In another embodiment, the first or second primers are complementary or substantially complementary (i.e., at least 70%, 75%, 80%, 85%, 90%, 95% or 99% complementary across at least 5, at least 10, at least 15, at least 20 or more nucleotides) to a framework 1, framework 2, or framework 3 region of an immune cell receptor V gene. In another embodiment, the first or second primers are complementary across their full-length to a framework 1, framework 2, or framework 3 region of an immune cell receptor V gene.

In yet another embodiment, the first or second primers are complementary or substantially complementary (i.e., at least 70%, 75%, 80%, 85%, 90%, 95% or 99% complementary) to an immune cell receptor J gene region. In another embodiment, the first or second primers are complementary or substantially complementary (i.e., at least 70%, 75%, 80%, 85%, 90%, 95% or 99% complementary across at least 5, at least 10, at least 15, at least 20 or more nucleotides) to an immune cell receptor J gene region. In another embodiment, the first or second primers are complementary across their full-length to an immune cell receptor J gene region.

In another embodiment, the first or second primers are complementary or substantially complementary (i.e., at least 70%, 75%, 80%, 85%, 90%, 95% or 99% complementary) to an immune cell receptor C gene region. In another embodiment, the first or second primers are complementary or substantially complementary (i.e., at least 70%, 75%, 80%, 85%, 90%, 95% or 99% complementary across at least 5, at least 10, at least 15, at least 20 or more nucleotides) to an immune cell receptor C gene region. In another embodiment, the first or second primers are complementary across their full-length to an immune cell receptor C gene region.

In some embodiments, genomic DNA or cDNA comprising target polynucleotides of the invention can be identified under stringent hybridization conditions using the primer/probes sequences disclosed here (e.g., comprising any one or more of SEQ ID NOS:1-213). The following is an exemplary set of hybridization conditions performed on a thermocycler and is not limiting: a) denaturation of sample containing target polynucleotides at 98° C. for 2 minutes; b) hybridization or primers/probes at 60° C. for 20 minutes; c) extension of primers/probes using polymerase at 65° C. for 2 minutes; d) addition of Exonuclease I to reaction mixture at 37° C. for 10 minutes, increase temperature to 80° C. for a further 10 minutes, hold at 4° C.

In one embodiment, target polynucleotides that selectively hybridize to any one of the primer/probe sequences disclosed herein (e.g., SEQ ID NOS: 1-213) can be of any length, e.g., at least 10, 15, 20, 25, 30, 50, 100, 200, 500 or more nucleotides or having fewer than 500, 200, 100, or 50 nucleotides, etc.).

In one embodiment, a first primer is hybridized to a region of a target immune cell receptor polynucleotide that is 3′ to a region of interest or at a 3′ end of a region of interest. For example, the region of interest can include at least a portion of the immune cell receptor V region, at least a portion of the C region, the diversity (D) region if present, and at least a portion of the J region. In some cases, the first primer hybridizes to a framework region (e.g., a framework 1, framework 2, or framework 3 region) of the immune cell receptor V region. In some cases, the first primer hybridizes to the immune cell receptor C region (e.g., comprising a C-segment primer). The first primer can then be extended by a polymerase in a first extension reaction. Un-extended first primers and other single-stranded nucleic acid (e.g., denatured genomic DNA) can then be removed, e.g., by single-stranded DNA exonuclease digestion. In a second reaction, a second primer hybridizes to a region of the extended first primer that is 3′ to the region of interest or at a 3′ end of a region of interest. Thus, the first and second primers flank the region of interest when hybridized to their respective targets. In some cases, the second primer hybridizes to a J-gene region of the extended first primer. In some cases, the second primer hybridizes to a V-gene region of the extended first primer. In some cases, the second primer hybridizes to a C gene region of the extended first primer. The second primer can then be extended by a polymerase in a second extension reaction.

Alternatively, a first primer can be hybridized to a J-gene region of a target immune cell receptor polynucleotide that is 3′ to a region of interest or at a 3′ end of a region of interest. The hybridized first primer can then be extended by a polymerase. Un-extended first primers and other single-stranded nucleic acid (e.g., denatured genomic DNA) can then be removed, e.g., by single-stranded DNA exonuclease digestion. In a second reaction, a second primer can be hybridized to a framework region of the extended first primer and extended with a polymerase.

In one embodiment, mRNA purified from a sample (e.g., cell, cell lysate or tissue) can be primed with an oligo-dT primer (e.g., a poly-T primer), random primer mixture (for example, a mixture or random hexamers, heptamers, octamers, nanomers, etc.,) or one or more isotype-specific immune receptor Constant-region (C-segment) primers (e.g., comprising SEQ ID NOS:205-213) under hybridization conditions sufficient to initiate cDNA synthesis. In one aspect, the oligo-dT primer facilitates the 3′ end of the mRNA's in the sample being represented in the resulting cDNA molecules. In another aspect, the one or more isotype-specific immune receptor C-segment primers allows for hybridization of the C-segment primer to a complementary sequence among the one or more mRNA transcripts present in the sample. In one embodiment, the resulting cDNA molecules can be used in one or more of the PETE assays as described herein to produce one or more primer extension products that can be used to identify immune receptor isotypes in the sample. Accordingly, RNA or mRNA molecules of the sample can undergo first, and preferably, second strand synthesis to produce double-stranded cDNA molecules. In one embodiment, cDNA is prepared using an oligo-dT primer as set forth in this paragraph. In another embodiment, cDNA is prepared using a random primer mixture of hexamers, heptamers, octamers, nanomers, etc., as set forth in this paragraph. In another embodiment, cDNA is prepared using at least one isotype-specific immune receptor C-segment primer. In another embodiment, cDNA can be prepared using one or more C-gene region primers. For example Glanville et al., (PNAS, (2009) 106.48, 20216-21) discloses using a human heavy chain constant region primer, human kappa constant region primer and human lambda constant region primer to prepare cDNA from total RNA and/or mRNA from human samples. In yet another embodiment, cDNA is prepared using at least one of the isotype-specific immune receptor C-segment primers set forth in SEQ ID NOS:205-213.

Once prepared, the synthesized cDNA can be purified by any method known in the art (e.g., Solid Phase Reversible Immobilization (SPRI) paramagnetic beads or AMPure paramagnetic beads (Beckman Coulter, Brea, CA), filtration or centrifugation columns (e.g., RNeasy Mini Kit (Catalog Number: 74104), Qiagen, Germantown, MD). The purified cDNA can then serve as a template for any of the methods described herein. In one embodiment, a plurality of V gene specific primers (e.g., comprising SEQ ID NOS:1-121) can be used in the first round of primer extension (gPE extension), followed by a second round of extension using one or more C-segment primers (e.g., comprising SEQ ID NOS:205-213). Alternatively, cDNA can be prepared where a plurality of V gene specific primers (e.g., comprising SEQ ID NOS:1-121) can be used in the first round of primer extension (gPE extension), followed by a second round of extension (PE2 extension) using a plurality of J-gene specific primers (e.g., comprising SEQ ID NOS:122-204).

Compositions for performing immunoPETE can include one or more, or all, of the following: primers, primer sets, polymerase extension products of such primers or primer sets (e.g., hybridized to a target polynucleotide), target polynucleotides (e.g., single-stranded target polynucleotides), reaction mixtures, DNA-dependent DNA polymerases, RNA-dependent DNA polymerases, single-stranded DNA exonucleases, nucleotides, buffers, salts, and the like.

In one embodiment, a composition containing a plurality of first primers is provided. In some cases, the plurality of first primers are immune cell receptor gene specific primers. The plurality of immune cell receptor gene specific primers can be configured to hybridize to, and thus enrich in a polymerase-mediated extension step, a plurality of target polynucleotides in a sample that encode immune cell receptor genes. The first primers can include a 5′-terminal phosphate (“[5′-Phos]”) when subsequent ligation will be performed, but in other embodiments ligation is not performed and the primers lack the 5′-Phos. The 5′-terminal phosphate can allow for ligation of the 5′ end of the first primer, or a polymerase extension product thereof, to a 3′-OH of an adjacent polynucleotide.

The first primers can include a [SPLINT] region. The [SPLINT] region can include an adapter hybridization site of at least 2 nucleotides in length. In some cases, the adapter hybridization site is at least 4 nucleotides in length, at least 6 nucleotides in length, at least 8 nucleotides in length, from 2 to 10 nucleotides in length, or from 2 to 8 nucleotides in length. The [SPLINT] region can be complementary to a single-stranded 5′ overhang region of a double-stranded adapter, such that when the single-stranded 5′ overhang region of the double-stranded adapter hybridizes to the [SPLINT] region, a 3′-OH of the adapter can be ligated to the [5′-Phos] of the first primer or a polymerase extension product thereof. In some embodiments, [SPLINT] comprises or consists of 6 consecutive nucleotides that are complementary to a single-stranded 5′ overhang region of a double-stranded adapter. In some embodiments, [SPLINT] comprises or consists of the sequence CGA TCT.

The first primers can include a [BARCODE] region that is or contains a barcode. The [BARCODE] region can be or contain a UID, an MID, or a combination thereof. In some cases, the [BARCODE] region comprises a UID. The [BARCODE] region can be any length from 2 to about 50 or more nucleotides. For UID barcodes, generally, the barcode length and composition is selected to encode more sequences than there are unique target polynucleotides to barcode. As such, for immune repertoire profiling, where estimates of diversity are generally significantly greater than 10³ and each unique sequence can be represented in a sample multiple times, the UID barcode can be at least 5, 6, 7, 8, 9, 10, 11, 12, 13, 14, 15, or 16 nucleotides in length. For example, in some cases the barcode can be from 6 to 16 nucleotides in length, from 8 to 14 nucleotides in length, or from 10 to 13 nucleotides in length. In some embodiments, the barcode is 13 nucleotides in length. In some cases, the barcode comprises nucleotides selected from the group consisting of N and W. In an exemplary embodiment, the barcode has a sequence of WNN NNN WNN NNN W.

The first primers can include a [FW] region. The [FW] region can be or contain a structurally distinct sequence that specifically hybridizes to a framework 1, framework 2, or framework 3 region of an immune cell receptor V gene region of an immune cell receptor (TCR or BCR) encoding target polynucleotide, wherein upon hybridization to the V gene region of an immune cell receptor encoding target polynucleotide, the first primer can be extended in the direction of the J gene region of the immune cell receptor encoding target polynucleotide. In some embodiments, the [FW] region hybridizes to a framework 1, framework 2, or framework 3 region of an TCR encoding target polynucleotide. In some embodiments, the [FW] region hybridizes to a framework 1, framework 2, or framework 3 region of a BCR encoding target polynucleotide. First primers containing a [FW] region can be used in an immunoPETE method with second primers that hybridize to the J gene region of an immune cell receptor encoding target polynucleotide. In some embodiments, the first primers include the following regions: [5′-Phos], [SPLINT], [BARCODE], and [FW]. In some embodiments, the first primers include the following regions from 5′ to 3′: [5′-Phos], [SPLINT], [BARCODE], and [FW].

Alternatively, the first primers can include a J-specific region ([J]) that specifically hybridizes to a J gene region of an immune cell receptor encoding target polynucleotide, wherein upon hybridization to the J gene region of an immune cell receptor (TCR or BCR) encoding target polynucleotide, the first primer can be extended in the direction of the V gene region of the immune cell receptor encoding target polynucleotide. In some embodiments, the [J] region hybridizes to a J gene region of a TCR encoding target polynucleotide. In some embodiments, the [J] region hybridizes to a J gene region of a BCR encoding target polynucleotide. First primers containing a region [J] can be used in an immunoPETE method with second primers that hybridize to the framework 1, framework 2, or framework 3 region of an immune cell receptor encoding target polynucleotide. In some embodiments, the first primers include the following regions: [5′-Phos], [SPLINT], [BARCODE], and [J]. In some embodiments, the first primers include the following regions from 5′ to 3′: [5′-Phos], [SPLINT], [BARCODE], and [J]. In another embodiment, the first primers can include a Constant-specific region ([C]) that specifically hybridizes to a C gene region of an immune cell receptor encoding target polynucleotide, wherein upon hybridization to the C gene region of an immune cell receptor (TCR or BCR) encoding target polynucleotide, the first primer can be extended in a 3′ direction, through the C gene region of the immune cell receptor encoding target polynucleotide. In some embodiments, the [C] region hybridizes to a C gene region of a TCR encoding target polynucleotide. In some embodiments, the [C] region hybridizes to a C gene region of a BCR encoding target polynucleotide. In some embodiments, first primers containing a C-region [C] can be used in an immunoPETE method with second primers that hybridize to the V-specific region ([V]) that specifically hybridizes to a V gene region of an immune cell receptor encoding target polynucleotide. In some embodiments, the first primers include the following regions: [5′-Phos], [SPLINT], [BARCODE], and [C]. In some embodiments, where the sample includes purified mRNA or total RNA, the first primers can include a C-segment primer that is complementary to, and hybridizes under hybridization conditions with a C gene region of an immune cell receptor encoding polynucleotide, or a complement thereof.

In some embodiments, target polynucleotides encoding a TCR (3-chain or 6-chain can contain a D region that is positioned between a V region and a J region, whereas target polynucleotides encoding a TCR α-chain or γ-chain can lack a D region, such that the V region and J region are adjacent. Similarly, target polynucleotides encoding a BCR heavy chain can contain a D region that is positioned between a V region and a J region, whereas target polynucleotides encoding a BCR light chain can lack a D region, such that the V region and J region are adjacent.

As such, first primer extension products of first primers containing an [FW] region that are templated by a TCR (3-chain or 6-chain or BCR heavy chain can, e.g., contain a VDJ region, or a portion of the V region, a D region, and a J region. Similarly, first primer extension products of first primers containing an [FW] region that are templated by a TCR α-chain or γ-chain or a BCR light chain can lack a D region, and thus contain a V region or portion thereof adjacent to a J region. Alternatively, first primer extension products of first primers containing a [J] region that are templated by a TCR (3-chain or 6-chain or BCR heavy chain can, e.g., contain a VDJ region, or a portion of the J region, a D region, and a V region. Similarly, first primer extension products of first primers containing a [J] region that are templated by a TCR α-chain or γ-chain or a BCR light chain can lack a D region, and thus contain a J region or portion thereof adjacent to a V region.

In some embodiments, the plurality of first primers contains at least 2, at least 5, at least 10, at least 25, at least 50, at least 100, or all of the primers set forth in SEQ ID Nos: 1-121, which may comprise or lack the 5′Phos moiety depending on whether they are used in an immunoPETE method involving or excluding ligation, respectively. In one embodiment, the plurality of first primers contains at least 1 of the primers set forth in SEQ ID Nos: 1-121. In some embodiments, the plurality of first primers contains at least 2, at least 5, at least 10, or all of the primers set forth in SEQ ID NOS: 205-213. In one embodiment, the plurality of first primers contains at least 1 of the primers set forth in SEQ ID NOS: 205-213. In one embodiment, the plurality of first primers are complementary or substantially complementary (i.e., at least 70%, 75%, 80%, 85%, 90%, 95% or 99% complementary) to an immune cell receptor C gene region. In another embodiment, the first primers are complementary or substantially complementary (i.e., at least 70%, 75%, 80%, 85%, 90%, 95% or 99% complementary across at least 5, at least 10, at least 15, at least 20 or more nucleotides) to an immune cell receptor C gene region. In another embodiment, the first primers are complementary across their full-length to an immune cell receptor C gene region.

TABLE 1

| SEQ ID NO: 1. | [5′-Phos]-[SPLINT]-[BARCODE]-GA ATG CCC TGA CAG CTC TCG CTT ATA |
|---|---|
| SEQ ID NO: 2. | [5′-Phos]-[SPLINT]-[BARCODE]-CT CAG AGA AGT CTG AAA TAT TCG ATG ATC |
| | AAT TCT CAG TTG |
| SEQ ID NO: 3. | [5′-Phos]-[SPLINT]-[BARCODE]-CC AAA TCG MTT CTC ACC TAA ATC TCC AGA |
| | CAA AG |
| SEQ ID NO: 4. | [5′-Phos]-[SPLINT]-[BARCODE]-CA CCT GAC TCT CCA GAC AAA GCT CAT |
| SEQ ID NO: 5. | [5′-Phos]-[SPLINT]-[BARCODE]-CC TGA ATG CCC CAA CAG CTC TC |
| SEQ ID NO: 6. | [5′-Phos]-[SPLINT]-[BARCODE]-GA TTC TCA GGG CGC CAG TTC TCT A |
| SEQ ID NO: 7. | [5′-Phos]-[SPLINT]-[BARCODE]-CC TAA TTG ATT CTC AGC TCA CCA CGT CCA TA |
| SEQ ID NO: 8. | [5′-Phos]-[SPLINT]-[BARCODE]-TC AGG GCG CCA GTT CCA TG |
| SEQ ID NO: 9. | [5′-Phos]-[SPLINT]-[BARCODE]-TC CTA GAT TCT CAG GTC TCC AGT TCC CTA |
| SEQ ID NO: 10. | [5′-Phos]-[SPLINT]-[BARCODE]-GA GGA AAC TTC CCT GAT CGA TTC TCA GC |
| SEQ ID NO: 11. | [5′-Phos]-[SPLINT]-[BARCODE]-CA ACT TCC CTG ATC GAT TCT CAG GTC A |
| SEQ ID NO: 12. | [5′-Phos]-[SPLINT]-[BARCODE]-AG GAA ACT TCC CTG ATC AAT TCT CAG GTC A |
| SEQ ID NO: 13. | [5′-Phos]-[SPLINT]-[BARCODE]-GG AAA CTT CCC TCC TAG ATT TTC AGG TCG |
| SEQ ID NO: 14. | [5′-Phos]-[SPLINT]-[BARCODE]-CC CCA ATG GCT ACA ATG TCT CCA GAT T |
| SEQ ID NO: 15. | [5′-Phos]-[SPLINT]-[BARCODE]-GG AGA GGT CCC TGA TGG CTA CAA |
| SEQ ID NO: 16. | [5′-Phos]-[SPLINT]-[BARCODE]-TC CCT GAT GGT TAT AGT GTC TCC AGA GC |
| SEQ ID NO: 17. | [5′-Phos]-[SPLINT]-[BARCODE]-GG AGA AGT CCC CAA TGG CTA CAA TGT C |
| SEQ ID NO: 18. | [5′-Phos]-[SPLINT]-[BARCODE]-AA AGG AGA AGT CCC GAA TGG CTA CAA |
| SEQ ID NO: 19. | [5′-Phos]-[SPLINT]-[BARCODE]-GT TCC CAA TGG CTA CAA TGT CTC CAG ATC |
| SEQ ID NO: 20. | [5′-Phos]-[SPLINT]-[BARCODE]-GA AGT CCC CAA TGG CTA CAA TGT CTC TAG |
| | ATT |
| SEQ ID NO: 21. | [5′-Phos]-[SPLINT]-[BARCODE]-GA GAA GTC CCC GAT GGC TAC AAT GTA |
| SEQ ID NO: 22. | [5′-Phos]-[SPLINT]-[BARCODE]-GT GAT CGG TTC TCT GCA CAG AGG T |
| SEQ ID NO: 23. | [5′-Phos]-[SPLINT]-[BARCODE]-CG CTT CTC TGC AGA GAG GAC TGG |
| SEQ ID NO: 24. | [5′-Phos]-[SPLINT]-[BARCODE]-GG TTC TTT GCA GTC AGG CCT GA |
| SEQ ID NO: 25. | [5′-Phos]-[SPLINT]-[BARCODE]-CA GTG GTC GGT TCT CTG CAG AG |
| SEQ ID NO: 26. | [5′-Phos]-[SPLINT]-[BARCODE]-GC TCA GTG ATC AAT TCT CCA CAG AGA GGT |
| SEQ ID NO: 27. | [5′-Phos]-[SPLINT]-[BARCODE]-TT CTC TGC AGA GAG GCC TGA GG |
| SEQ ID NO: 28. | [5′-Phos]-[SPLINT]-[BARCODE]-CC CAG TGA TCG CTT CTT TGC AGA AA |
| SEQ ID NO: 29. | [5′-Phos]-[SPLINT]-[BARCODE]-CT GCA GAG AGG CCT AAG GGA TCT |
| SEQ ID NO: 30. | [5′-Phos]-[SPLINT]-[BARCODE]-GA AGG GTA CAA TGT CTC TGG AAA CAA ACT |
| | CAAG |
| SEQ ID NO: 31. | [5′-Phos]-[SPLINT]-[BARCODE]-GG GGT ACT GTG TTT CTT GAA ACA AGC TTG |
| | AG |
| SEQ ID NO: 32. | [5′-Phos]-[SPLINT]-[BARCODE]-CA GTT CCC TGA CTT GCA CTC TGA ACT AAA C |
| SEQ ID NO: 33. | [5′-Phos]-[SPLINT]-[BARCODE]-AC TAA CAA AGG AGA AGT CTC AGA TGG CTA |
| | CAG |
| SEQ ID NO: 34. | [5′-Phos]-[SPLINT]-[BARCODE]-AG ATA AAG GAG AAG TCC CCG ATG GCT A |
| SEQ ID NO: 35. | [5′-Phos]-[SPLINT]-[BARCODE]-GA TAC TGA CAA AGG AGA AGT CTC AGA TGG |
| | CTA TAG |
| SEQ ID NO: 36. | [5′-Phos]-[SPLINT]-[BARCODE]-CT AAG GAT CGA TTT TCT GCA GAG AGG CTC |
| SEQ ID NO: 37. | [5′-Phos]-[SPLINT]-[BARCODE]-TT GAT TCT CAG CAC AGA TGC CTG ATG T |
| SEQ ID NO: 38. | [5′-Phos]-[SPLINT]-[BARCODE]-AT TCT CAG CTG AGA GGC CTG ATG G |
| SEQ ID NO: 39. | [5′-Phos]-[SPLINT]-[BARCODE]-GG ATC GAT TCT CAG CTA AGA TGC CTA ATG C |
| SEQ ID NO: 40. | [5′-Phos]-[SPLINT]-[BARCODE]-CT CAG CAG AGA TGC CTG ATG CAA CTT TA |
| SEQ ID NO: 41. | [5′-Phos]-[SPLINT]-[BARCODE]-CT GAT CGA TTC TCA GCT CAA CAG TTC AGT |
| SEQ ID NO: 42. | [5′-Phos]-[SPLINT]-[BARCODE]-TA GCT GAA AGG ACT GGA GGG ACG TAT |
| SEQ ID NO: 43. | [5′-Phos]-[SPLINT]-[BARCODE]-CC AGG AGG CCG AAC ACT TCT TTC T |
| SEQ ID NO: 44. | [5′-Phos]-[SPLINT]-[BARCODE]-GC TAA GTG CCT CCC AAA TTC ACC CT |
| SEQ ID NO: 45. | [5′-Phos]-[SPLINT]-[BARCODE]-CA CAG CTG AAA GAC CTA ACG GAA CGT |
| SEQ ID NO: 46. | [5′-Phos]-[SPLINT]-[BARCODE]-CT GCT GAA TTT CCC AAA GAG GGC C |
| SEQ ID NO: 47. | [5′-Phos]-[SPLINT]-[BARCODE]-AG GGT ACA GCG TCT CTC GGG |
| SEQ ID NO: 48. | [5′-Phos]-[SPLINT]-[BARCODE]-GC CTG ACC TTG TCC ACT CTG ACA |
| SEQ ID NO: 49. | [5′-Phos]-[SPLINT]-[BARCODE]-AT GAG CGA TTT TTA GCC CAA TGC TCC A |
| SEQ ID NO: 50. | [5′-Phos]-[SPLINT]-[BARCODE]-TG AAG GCT ACG TGT CTG CCA AGA G |
| SEQ ID NO: 51. | [5′-Phos]-[SPLINT]-[BARCODE]-CT CAT CTC AAT GCC CCA AGA ACG C |
| SEQ ID NO: 52. | [5′-Phos]-[SPLINT]-[BARCODE]-AG ATC TCT GAT GGA TAC AGT GTC TCT CGA |
| | CA |
| SEQ ID NO: 53. | [5′-Phos]-[SPLINT]-[BARCODE]-AG ATC TTT CCT CTG AGT CAA CAG TCT CCA |
| | GAA TA |
| SEQ ID NO: 54. | [5′-Phos]-[SPLINT]-[BARCODE]-CA CTG AAA AAG GAG ATA TCT CTG AGG GGT |
| | ATC ATG |
| SEQ ID NO: 55. | [5′-Phos]-[SPLINT]-[BARCODE]-GT TCC TGA AGG GTA CAA AGT CTC TCG AAA |
| | AG |
| SEQ ID NO: 56. | [5′-Phos]-[SPLINT]-[BARCODE]-CT GAG GGG TAC AGT GTC TCT AGA GAG A |
| SEQ ID NO: 57. | [5′-Phos]-[SPLINT]-[BARCODE]-AG CCG CCC AAA CCT AAC ATT CTC AA |
| SEQ ID NO: 58. | [5′-Phos]-[SPLINT]-[BARCODE]-CC CAG GAC CGG CAG TTC A |
| SEQ ID NO: 59. | [5′-Phos]-[SPLINT]-[BARCODE]-TT GAT TAG AGA CAT ATC CCT ATT GAA AAT |
| | ATT TCC TGG CA |
| SEQ ID NO: 60. | [5′-Phos]-[SPLINT]-[BARCODE]-AG ATG CCC TGA GTC AGC ATA GTC ATT CTA |
| | AC |
| SEQ ID NO: 61. | [5′-Phos]-[SPLINT]-[BARCODE]-GG AGG GGA AGG CCC CAC AGC GTC TTC |
| SEQ ID NO: 62. | [5′-Phos]-[SPLINT]-[BARCODE]-TG AAG TCA TAC AGT TCC TGG TGT CCA T |
| SEQ ID NO: 63. | [5′-Phos]-[SPLINT]-[BARCODE]-CC AAA TCA GGC TTT GGA GCA CCT GAT CT |
| SEQ ID NO: 64. | [5′-Phos]-[SPLINT]-[BARCODE]-CC AAA CAA AGG CTT AGA ATA TTT ATT ACA |
| | TGT C |
| SEQ ID NO: 65. | [5′-Phos]-[SPLINT]-[BARCODE]-CC AGG TCC CTG AGG CAC TCC ACC AGC T |
| SEQ ID NO: 66. | [5′-Phos]-[SPLINT]-[BARCODE]-CT GAA TCT AAA TTA TGA GCC ATC TGA CA |
| SEQ ID NO: 67. | [5′-Phos]-[SPLINT]-[BARCODE]-TC ATT CCT TAG TCG CTC TGA TAG TTA TGG |
| | TTA |
| SEQ ID NO: 68. | [5′-Phos]-[SPLINT]-[BARCODE]-CA TTC CTT AGT CGG TCT AAA GGG TAC AGT |
| | TA |
| SEQ ID NO: 69. | [5′-Phos]-[SPLINT]-[BARCODE]-AC AAC ATG ACC TAT GAA CGG TTC TCT TCA |
| | TC |
| SEQ ID NO: 70. | [5′-Phos]-[SPLINT]-[BARCODE]-CT GAA TTT AAC AAG AGC CAA ACC TCC TTC |
| | CA |
| SEQ ID NO: 71. | [5′-Phos]-[SPLINT]-[BARCODE]-CC GAC AGA AAG TCC AGC ACT CTG AG |
| SEQ ID NO: 72. | [5′-Phos]-[SPLINT]-[BARCODE]-CA CTG TTC TAT TGA ATA AAA AGG ATA AAC |
| | ATC TGT C |
| SEQ ID NO: 73. | [5′-Phos]-[SPLINT]-[BARCODE]-GT CAC CTT TGA TAC CAC CCT TAA ACA GAG |
| | TTT |
| SEQ ID NO: 74. | [5′-Phos]-[SPLINT]-[BARCODE]-AG ACT AAA TGC TAC ATT ACT GAA GAA TGG |
| | AAG CAG |
| SEQ ID NO: 75. | [5′-Phos]-[SPLINT]-[BARCODE]-TG AGG CTG AAT TTA TAA AGA GTA AAT TCT |
| | CCT TTA A |
| SEQ ID NO: 76. | [5′-Phos]-[SPLINT]-[BARCODE]-GC TGA ATT TAA GAA GAG TGA AAC CTC CTT |
| | CCA |
| SEQ ID NO: 77. | [5′-Phos]-[SPLINT]-[BARCODE]-GG CTG AAT TTA AGA GGA GTC AAT CTT CCT |
| | TCA A |
| SEQ ID NO: 78. | [5′-Phos]-[SPLINT]-[BARCODE]-GA CAC TTA TCA CTT CCC CAA TCA ATA CCC C |
| SEQ ID NO: 79. | [5′-Phos]-[SPLINT]-[BARCODE]-GG CTG AAT TTA ACA AGA GTC AAA CTT CCT |
| | TCC A |
| SEQ ID NO: 80. | [5′-Phos]-[SPLINT]-[BARCODE]-GC TGA ATT TAA GAA GAG CGA AAC CTC CTT |
| | CTA |
| SEQ ID NO: 81. | [5′-Phos]-[SPLINT]-[BARCODE]-CC ATG TAC CGT AAA GAA ACC ACT TCT TTC |
| | CA |
| SEQ ID NO: 82. | [5′-Phos]-[SPLINT]-[BARCODE]-CC ACA TAC CGT AAA GAA ACC ACT TCT TTC |
| | CA |
| SEQ ID NO: 83. | [5′-Phos]-[SPLINT]-[BARCODE]-TG GAT GCA GAC ACA AAG CAA AGC TC |
| SEQ ID NO: 84. | [5′-Phos]-[SPLINT]-[BARCODE]-TA AAG AAC TGC TTG GAA AAG AAA AAT TTT |
| | ATA GTGT |
| SEQ ID NO: 85. | [5′-Phos]-[SPLINT]-[BARCODE]-AC AGC TCA ATA GAG CCA GCC AGT ATA TTT C |
| SEQ ID NO: 86. | [5′-Phos]-[SPLINT]-[BARCODE]-CA GCT CAA TAA AGC CAG CCA GTA TGT TTC |
| SEQ ID NO: 87. | [5′-Phos]-[SPLINT]-[BARCODE]-GC ACA GGT CGA TAA ATC CAG CAA GTA TAT |
| | CTC |
| SEQ ID NO: 88. | [5′-Phos]-[SPLINT]-[BARCODE]-CT GTT ACA TTG AAC AAG ACA GCC AAA CAT |
| | TTC TC |
| SEQ ID NO: 89. | [5′-Phos]-[SPLINT]-[BARCODE]-CA CCG TTT TAT TGA ATA AGA CAG TGA AAC |
| | ATC TCT C |
| SEQ ID NO: 90. | [5′-Phos]-[SPLINT]-[BARCODE]-CC AGA AGG CAA GAA AAT CCG CCA A |
| SEQ ID NO: 91. | [5′-Phos]-[SPLINT]-[BARCODE]-AG AAG CGC TTG GAA AAG AGA AGT TTT ATA |
| | GTGT |
| SEQ ID NO: 92. | [5′-Phos]-[SPLINT]-[BARCODE]-TG ACC TTA ACA AAG GCG AGA CAT CTT TCC A |
| SEQ ID NO: 93. | [5′-Phos]-[SPLINT]-[BARCODE]-CG CTT GAC ACT TCC AAG AAA AGC AGT TC |
| SEQ ID NO: 94. | [5′-Phos]-[SPLINT]-[BARCODE]-CA GTC CTA TCA AGA GTG ACA GTT CCT TCC A |
| SEQ ID NO: 95. | [5′-Phos]-[SPLINT]-[BARCODE]-GA ACT TCC AGA AAT CCA CCA GTT CCT TCA A |
| SEQ ID NO: 96. | [5′-Phos]-[SPLINT]-[BARCODE]-GC TAA AAG CCA CAT TAA CAA AGA AGG AAA |
| | GCT T |
| SEQ ID NO: 97. | [5′-Phos]-[SPLINT]-[BARCODE]-CT CGC TGG ATA AAT CAT CAG GAC GTA GTA C |
| SEQ ID NO: 98. | [5′-Phos]-[SPLINT]-[BARCODE]-TC GCT ACG GAA CGC TAC AGC TT |
| SEQ ID NO: 99. | [5′-Phos]-[SPLINT]-[BARCODE]-CT CCT TCA ATA AAA GTG CCA AGC AGT TCT C |
| SEQ ID NO: | [5′-Phos]-[SPLINT]-[BARCODE]-CA CTC TTA ATA CCA AGG AGG GTT ACA GCT A |
| 100. | |
| SEQ ID NO: | [5′-Phos]-[SPLINT]-[BARCODE]-TC AGT TTG GAG AAG CAA AAA AGA ACA GCT C |
| 101. | |
| SEQ ID NO: | [5′-Phos]-[SPLINT]-[BARCODE]-GA TCA TCA CAG AAG ACA GAA AGT CCA GCA |
| 102. | C |
| SEQ ID NO: | [5′-Phos]-[SPLINT]-[BARCODE]-GC AAT CGC TGA AGA CAG AAA GTC CAG TAC |
| 103. | |
| SEQ ID NO: | [5′-Phos]-[SPLINT]-[BARCODE]-CA GTT TGG TGA TGC AAG AAA GGA CAG TTC |
| 104. | |
| SEQ ID NO: | [5′-Phos]-[SPLINT]-[BARCODE]-CA GTC AAA GCT GAG GAA CTT TAT GGC CA |
| 105. | |
| SEQ ID NO: | [5′-Phos]-[SPLINT]-[BARCODE]-CT TCT TAA ACA AAA GTG CCA AGC ACC TCT C |
| 106. | |
| SEQ ID NO: | [5′-Phos]-[SPLINT]-[BARCODE]-CT GCT TCA TTT AAT GAA AAA AAG CAG CAA |
| 107. | AGC TC |
| SEQ ID NO: | [5′-Phos]-[SPLINT]-[BARCODE]-TT CTG TGA GCT TCC AGA AAA CAA CTA AAA |
| 108. | CTA TTC A |
| SEQ ID NO: | [5′-Phos]-[SPLINT]-[BARCODE]-CA CTG TAC TGT TGA ATA AAA ATG CTA AAC |
| 109. | ATG TCT C |
| SEQ ID NO: | [5′-Phos]-[SPLINT]-[BARCODE]-GC CTG TGA ACT TTG AAA AAA AGA AAA AGT |
| 110. | TCA TCA A |
| SEQ ID NO: | [5′-Phos]-[SPLINT]-[BARCODE]-CC AAG TTG GAT GAG AAA AAG CAG CAA AGT |
| 111. | TC |
| SEQ ID NO: | [5′-Phos]-[SPLINT]-[BARCODE]-TC AGT TTG GTA TAA CCA GAA AGG ACA GCT T |
| 112. | |
| SEQ ID NO: | [5′-Phos]-[SPLINT]-[BARCODE]-AA GTA GCA TAT TAG ATA AGA AAG AAC TTT |
| 113. | CCA GCA T |
| SEQ ID NO: | [5′-Phos]-[SPLINT]-[BARCODE]-CA GGC TTA AAA AAG GAG ACC AGC ACA TTT C |
| 114. | |
| SEQ ID NO: | [5′-Phos]-[SPLINT]-[BARCODE]-CT TCC AGA AAG CAG CCA AAT CCT TCA G |
| 115. | |
| SEQ ID NO: | [5′-Phos]-[SPLINT]-[BARCODE]-TG ATA CCA AAG CCC GTC TCA GCA C |
| 116. | |
| SEQ ID NO: | [5′-Phos]-[SPLINT]-[BARCODE]-GA GGC GGA AAT ATT AAA GAC AAA AAC TCC |
| 117. | CC |
| SEQ ID NO: | [5′-Phos]-[SPLINT]-[BARCODE]-CC ACA ATA AAC ATA CAG GAA AAG CAC AGC |
| 118. | TC |
| SEQ ID NO: | [5′-Phos]-[SPLINT]-[BARCODE]-AG AAA GCA GCG AAA TCC GTC GC |
| 119. | |
| SEQ ID NO: | [5′-Phos]-[SPLINT]-[BARCODE]-TG ACA TTG ATA TTG CAA AGA ACC TGG CTG T |
| 120. | |
| SEQ ID NO: | [5′-Phos]-[SPLINT]-[BARCODE]-GA AAC ACA TTC TGA CCC AGA AAG CCT TTC A |
| 121. | |

In one embodiment, a composition containing a plurality of second primers is provided. In some cases, the plurality of second primers are immune cell receptor gene-specific primers. The plurality of immune cell receptor gene-specific primers can be configured to hybridize to, and thus enrich in a polymerase-mediated extension step, a plurality of target polynucleotides in a sample that encode immune cell receptor genes. The second primers can include a universal primer binding site, or complement thereof, and an immune cell receptor hybridizing region. In some cases, the second primers include from 5′ to 3′ a universal primer binding site and an immune cell receptor hybridizing region. In some cases, the second primer immune cell receptor hybridizing region is complementary to, and hybridizes under hybridization conditions with, a J-gene region of an immune cell receptor encoding polynucleotide, or a complement thereof. In some cases, the second primer immune cell receptor hybridizing region is complementary to, and hybridizes under hybridization conditions with, a V region of an immune cell receptor encoding polynucleotide (e.g., a framework 1, framework 2, or framework 3 region), or a complement thereof. In some cases, the second primer immune cell receptor hybridizing region is complementary to, and hybridizes under hybridization conditions with, a C gene region of an immune cell receptor encoding polynucleotide, or a complement thereof.

As described herein, target polynucleotides encoding a TCR (3-chain or 6-chain can contain a D region that is positioned between a V region and a J region, whereas target polynucleotides encoding a TCR α-chain or γ-chain can lack a D region, such that the V region and J region are adjacent. Similarly, target polynucleotides encoding a BCR heavy chain can contain a D region that is positioned between a V region and a J region, whereas target polynucleotides encoding a BCR light chain can lack a D region, such that the V region and J region are adjacent.

Second primer extension products of second primers containing a [J] or an [FW] region that are templated by a first primer extension product templated by a TCR (3-chain or 6-chain or BCR heavy chain can, e.g., contain a VDJ region, or a portion of the V region, a D region, and a portion of a J region. Similarly, second primer extension products of second primers containing a [J] or an [FW] region that are templated by first primer extension product templated by a TCR α-chain or γ-chain or a BCR light chain can lack a D region, and thus contain a V region or portion thereof adjacent to a J region or portion thereof.

In some embodiments, the plurality of second primers contains at least 2, at least 5, at least 10, at least 25, at least 50, at least 75, or all of the primers set forth in SEQ ID Nos: 122-204. In one embodiment, the plurality of second primers contains at least 1 of the primers set forth in SEQ ID Nos: 122-204. In some embodiments, the plurality of second primers contains at least 2, at least 5, at least 10, or all of the primers set forth in SEQ ID NOS: 205-213. In one embodiment, the plurality of second primers contains at least 1 of the primers set forth in SEQ ID NOS: 205-213.

In one embodiment, the second primers are complementary or substantially complementary (i.e., at least 70%, 75%, 80%, 85%, 90%, 95% or 99% complementary) to a framework 1, framework 2, or framework 3 region of an immune cell receptor V gene. In another embodiment, the second primers are complementary or substantially complementary (i.e., at least 70%, 75%, 80%, 85%, 90%, 95% or 99% complementary across at least 5, at least 10, at least 15, at least 20 or more nucleotides) to a framework 1, framework 2, or framework 3 region of an immune cell receptor V gene. In another embodiment, the second primers are complementary across their full-length to a framework 1, framework 2, or framework 3 region of an immune cell receptor V gene.

In yet another embodiment, the second primers are complementary or substantially complementary (i.e., at least 70%, 75%, 80%, 85%, 90%, 95% or 99% complementary) to an immune cell receptor J gene region. In another embodiment, the second primers are complementary or substantially complementary (i.e., at least 70%, 75%, 80%, 85%, 90%, 95% or 99% complementary across at least 5, at least 10, at least 15, at least 20 or more nucleotides) to an immune cell receptor J gene region. In another embodiment, the second primers are complementary across their full-length to an immune cell receptor J gene region.

TABLE 2

| SEQ ID | |
|---|---|
| NO: | Sequence |
| SEQ ID | AAT GAT ACG GCG ACC ACC GAG ATC TAC ACT CTT TCC CTA CAC |
| NO: | GAC GCT CTT CCG ATC TGG GGA GAA GTG GAA ACT CTG GTT CC |
| 122. | |
| SEQ ID | AAT GAT ACG GCG ACC ACC GAG ATC TAC ACT CTT TCC CTA CAC |
| NO: | GAC GCT CTT CCG ATC TGA CTC ACC AGA TAT AAT GAA TAC ATG |
| 123. | GGT CCC |
| SEQ ID | AAT GAT ACG GCG ACC ACC GAG ATC TAC ACT CTT TCC CTA CAC |
| NO: | GAC GCT CTT CCG ATC TCC GGA TGC TGA GTC TGG TCC C |
| 124. | |
| SEQ ID | AAT GAT ACG GCG ACC ACC GAG ATC TAC ACT CTT TCC CTA CAC |
| NO: | GAC GCT CTT CCG ATC TTG TAC AGC CAG CCT GGT CCC |
| 125. | |
| SEQ ID | AAT GAT ACG GCG ACC ACC GAG ATC TAC ACT CTT TCC CTA CAC |
| NO: | GAC GCT CTT CCG ATC TCT TGG TTG CAC TTG GAG TCT TGT TCC |
| 126. | |
| SEQ ID | AAT GAT ACG GCG ACC ACC GAG ATC TAC ACT CTT TCC CTA CAC |
| NO: | GAC GCT CTT CCG ATC TCA CGG ATG AAC AAT AAG GCT GGT TCC |
| 127. | |
| SEQ ID | AAT GAT ACG GCG ACC ACC GAG ATC TAC ACT CTT TCC CTA CAC |
| NO: | GAC GCT CTT CCG ATC TTT GGT ATG ACC ACC ACT TGG TTC CC |
| 128. | |
| SEQ ID | AAT GAT ACG GCG ACC ACC GAG ATC TAC ACT CTT TCC CTA CAC |
| NO: | GAC GCT CTT CCG ATC TAC TGA CCA GAA GTC GGG TGC C |
| 129. | |
| SEQ ID | AAT GAT ACG GCG ACC ACC GAG ATC TAC ACT CTT TCC CTA CAC |
| NO: | GAC GCT CTT CCG ATC TAT GGA ACT TAC TTG CTT TAA CAA ATA GTC |
| 130. | TTG TTC C |
| SEQ ID | AAT GAT ACG GCG ACC ACC GAG ATC TAC ACT CTT TCC CTA CAC |
| NO: | GAC GCT CTT CCG ATC TAC TGA GTT CCA CTT TTA GCT GAG TGC C |
| 131. | |
| SEQ ID | AAT GAT ACG GCG ACC ACC GAG ATC TAC ACT CTT TCC CTA CAC |
| NO: | GAC GCT CTT CCG ATC TAC CTG GAG AGA CTA GAA GCA TAG TCC C |
| 132. | |
| SEQ ID | AAT GAT ACG GCG ACC ACC GAG ATC TAC ACT CTT TCC CTA CAC |
| NO: | GAC GCT CTT CCG ATC TCC TGA CCA GCA GTC TGG TCC C |
| 133. | |
| SEQ ID | AAT GAT ACG GCG ACC ACC GAG ATC TAC ACT CTT TCC CTA CAC |
| NO: | GAC GCT CTT CCG ATC TCT TGG GAT GAC TTG GAG CTT TGT TCC |
| 134. | |
| SEQ ID | AAT GAT ACG GCG ACC ACC GAG ATC TAC ACT CTT TCC CTA CAC |
| NO: | GAC GCT CTT CCG ATC TCC TAC TTA CCA GGT TTT ACT GAT AAT CTT |
| 135. | GTC CC |
| SEQ ID | AAT GAT ACG GCG ACC ACC GAG ATC TAC ACT CTT TCC CTA CAC |
| NO: | GAC GCT CTT CCG ATC TTA CTG GAA CTC ACT GAT AAG GTG GTT CC |
| 136. | |
| SEQ ID | AAT GAT ACG GCG ACC ACC GAG ATC TAC ACT CTT TCC CTA CAC |
| NO: | GAC GCT CTT CCG ATC TCT GGA ACT CAC TGA TAA GGT GGG TTC C |
| 137. | |
| SEQ ID | AAT GAT ACG GCG ACC ACC GAG ATC TAC ACT CTT TCC CTA CAC |
| NO: | GAC GCT CTT CCG ATC TTA CTT ACT AAG ATC CAC CTT TAA CAT GGT |
| 138. | TCC |
| SEQ ID | AAT GAT ACG GCG ACC ACC GAG ATC TAC ACT CTT TCC CTA CAC |
| NO: | GAC GCT CTT CCG ATC TAC TTG GTT TAA CTA GCA CCC TGG TTC C |
| 139. | |
| SEQ ID | AAT GAT ACG GCG ACC ACC GAG ATC TAC ACT CTT TCC CTA CAC |
| NO: | GAC GCT CTT CCG ATC TAG GCC AGA CAG TCA ACT GAG TTC C |
| 140. | |
| SEQ ID | AAT GAT ACG GCG ACC ACC GAG ATC TAC ACT CTT TCC CTA CAC |
| NO: | GAC GCT CTT CCG ATC TCT CAC TTA CTT GGA GTG ACA TTA TGT TTG |
| 141. | GAT CC |
| SEQ ID | AAT GAT ACG GCG ACC ACC GAG ATC TAC ACT CTT TCC CTA CAC |
| NO: | GAC GCT CTT CCG ATC TTA CTT ACT TGC TCT TAC AGT TAC TGT GGT |
| 142. | TCC |
| SEQ ID | AAT GAT ACG GCG ACC ACC GAG ATC TAC ACT CTT TCC CTA CAC |
| NO: | GAC GCT CTT CCG ATC TTA ACT TAC TTG GTT TTA CAT TGA GTT TGG |
| 143. | TCC C |
| SEQ ID | AAT GAT ACG GCG ACC ACC GAG ATC TAC ACT CTT TCC CTA CAC |
| NO: | GAC GCT CTT CCG ATC TTA CCA GGT AAA ACA GTC AAT TGT GTC CC |
| 144 | |
| SEQ ID | AAT GAT ACG GCG ACC ACC GAG ATC TAC ACT CTT TCC CTA CAC |
| NO: | GAC GCT CTT CCG ATC TAC TGG GTT TCA CAG ATA ACT CCG TTC C |
| 145. | |
| SEQ ID | AAT GAT ACG GCG ACC ACC GAG ATC TAC ACT CTT TCC CTA CAC |
| NO: | GAC GCT CTT CCG ATC TGG TGA CCA CAA CCT GGG TCC C |
| 146. | |
| SEQ ID | AAT GAT ACG GCG ACC ACC GAG ATC TAC ACT CTT TCC CTA CAC |
| NO: | GAC GCT CTT CCG ATC TGC TTG ACA AGC AGC CTT GTC CC |
| 147. | |
| SEQ ID | AAT GAT ACG GCG ACC ACC GAG ATC TAC ACT CTT TCC CTA CAC |
| NO: | GAC GCT CTT CCG ATC TGC AGC ACG GAC AAT CTG GTT CC |
| 148. | |
| SEQ ID | AAT GAT ACG GCG ACC ACC GAG ATC TAC ACT CTT TCC CTA CAC |
| NO: | GAC GCT CTT CCG ATC TGG CTT CAC AGT GAG CGT AGT CCC |
| 149. | |
| SEQ ID | AAT GAT ACG GCG ACC ACC GAG ATC TAC ACT CTT TCC CTA CAC |
| NO: | GAC GCT CTT CCG ATC TCT TGG TAT GAC CGA GAG TTT GGT CCC |
| 150. | |
| SEQ ID | AAT GAT ACG GCG ACC ACC GAG ATC TAC ACT CTT TCC CTA CAC |
| NO: | GAC GCT CTT CCG ATC TAC TTG CAA TCA CAG AAA GTC TTG TGC C |
| 151 | |
| SEQ ID | AAT GAT ACG GCG ACC ACC GAG ATC TAC ACT CTT TCC CTA CAC |
| NO: | GAC GCT CTT CCG ATC TCT GGG GAG AAT ATG AAG TCG TGT CCC |
| 152. | |
| SEQ ID | AAT GAT ACG GCG ACC ACC GAG ATC TAC ACT CTT TCC CTA CAC |
| NO: | GAC GCT CTT CCG ATC TGC TTC ACC ACC AGC TGA GTT CC |
| 153 | |
| SEQ ID | AAT GAT ACG GCG ACC ACC GAG ATC TAC ACT CTT TCC CTA CAC |
| NO: | GAC GCT CTT CCG ATC TCT GGA CAG CAA GCA GAG TGC C |
| 154 | |
| SEQ ID | AAT GAT ACG GCG ACC ACC GAG ATC TAC ACT CTT TCC CTA CAC |
| NO: | GAC GCT CTT CCG ATC TGA CTT ACC TGG CTT TAT AAT TAG CTT GGT |
| 155. | CCC |
| SEQ ID | AAT GAT ACG GCG ACC ACC GAG ATC TAC ACT CTT TCC CTA CAC |
| NO: | GAC GCT CTT CCG ATC TTA CTT ACT TGG AAA GAC TTG TAA TCT GGT |
| 156. | CCC |
| SEQ ID | AAT GAT ACG GCG ACC ACC GAG ATC TAC ACT CTT TCC CTA CAC |
| NO: | GAC GCT CTT CCG ATC TCT TAC GTG GTA AAA CAA TCA CTT GAG |
| 157. | TGC C |
| SEQ ID | AAT GAT ACG GCG ACC ACC GAG ATC TAC ACT CTT TCC CTA CAC |
| NO: | GAC GCT CTT CCG ATC TGG GGA ATA ACG GTG AGT CTC GTT CC |
| 158. | |
| SEQ ID | AAT GAT ACG GCG ACC ACC GAG ATC TAC ACT CTT TCC CTA CAC |
| NO: | GAC GCT CTT CCG ATC TCC TAC CTG GTT TTA CTT GGT AAA GTT GTC |
| 159. | CC |
| SEQ ID | AAT GAT ACG GCG ACC ACC GAG ATC TAC ACT CTT TCC CTA CAC |
| NO: | GAC GCT CTT CCG ATC TCG GAT TTA CTG CCA GGC TTG TTC C |
| 160. | |
| SEQ ID | AAT GAT ACG GCG ACC ACC GAG ATC TAC ACT CTT TCC CTA CAC |
| NO: | GAC GCT CTT CCG ATC TCG GGG TTT GAC CAT TAA CCT TGT TCC |
| 161. | |
| SEQ ID | AAT GAT ACG GCG ACC ACC GAG ATC TAC ACT CTT TCC CTA CAC |
| NO: | GAC GCT CTT CCG ATC TGC TAA AAC CTT CAG CCT GGT GCC |
| 162. | |
| SEQ ID | AAT GAT ACG GCG ACC ACC GAG ATC TAC ACT CTT TCC CTA CAC |
| NO: | GAC GCT CTT CCG ATC TGT GAC CAA CAG CGA GGT GCC |
| 163. | |
| SEQ ID | AAT GAT ACG GCG ACC ACC GAG ATC TAC ACT CTT TCC CTA CAC |
| NO: | GAC GCT CTT CCG ATC TAC ACT TAC TTG GTT TAA CAG AGA GTT |
| 164 | TAG TGC C |
| SEQ ID | AAT GAT ACG GCG ACC ACC GAG ATC TAC ACT CTT TCC CTA CAC |
| NO: | GAC GCT CTT CCG ATC TAC TTG GTT TTA CTG TCA GTC TGG TCC C |
| 165. | |
| SEQ ID | AAT GAT ACG GCG ACC ACC GAG ATC TAC ACT CTT TCC CTA CAC |
| NO: | GAC GCT CTT CCG ATC TCG AGC GTG ACC TGA AGT CTT GTT CC |
| 166 | |
| SEQ ID | AAT GAT ACG GCG ACC ACC GAG ATC TAC ACT CTT TCC CTA CAC |
| NO: | GAC GCT CTT CCG ATC TCA GGG CTG GAT GAT TAG ATG AGT CCC |
| 167. | |
| SEQ ID | AAT GAT ACG GCG ACC ACC GAG ATC TAC ACT CTT TCC CTA CAC |
| NO: | GAC GCT CTT CCG ATC TGG GCC TAA CTG CTA AAC GAG TCC C |
| 168. | |
| SEQ ID | AAT GAT ACG GCG ACC ACC GAG ATC TAC ACT CTT TCC CTA CAC |
| NO: | GAC GCT CTT CCG ATC TCA CAG GAC TTG ACT CTC AGA ATG GTT CC |
| 169 | |
| SEQ ID | AAT GAT ACG GCG ACC ACC GAG ATC TAC ACT CTT TCC CTA CAC |
| NO: | GAC GCT CTT CCG ATC TAC TGG GTA TGA TGG TGA GTC TTG TTC C |
| 170. | |
| SEQ ID | AAT GAT ACG GCG ACC ACC GAG ATC TAC ACT CTT TCC CTA CAC |
| NO: | GAC GCT CTT CCG ATC TCT TGG AAT GAC CGT CAA ACT TGT CCC |
| 171. | |
| SEQ ID | AAT GAT ACG GCG ACC ACC GAG ATC TAC ACT CTT TCC CTA CAC |
| | GAC GCT CTT CCG ATC TAC TTA CTT GGA ATG ACT GAT AAG CTT GTC |
| NO: | CC |
| 172. | |
| SEQ ID | AAT GAT ACG GCG ACC ACC GAG ATC TAC ACT CTT TCC CTA CAC |
| NO: | GAC GCT CTT CCG ATC TTG CTT GGC TTC ACA GTT AGT CAT GTC TC |
| 173. | |
| SEQ ID | AAT GAT ACG GCG ACC ACC GAG ATC TAC ACT CTT TCC CTA CAC |
| NO: | GAC GCT CTT CCG ATC TCT TGG ATG GAC AGT CAA GAT GGT CCC |
| 174. | |
| SEQ ID | AAT GAT ACG GCG ACC ACC GAG ATC TAC ACT CTT TCC CTA CAC |
| NO: | GAC GCT CTT CCG ATC TAA CTT ACT TGG ATT CAC GGT TAA GAG |
| 175. | AGT TCC |
| SEQ ID | AAT GAT ACG GCG ACC ACC GAG ATC TAC ACT CTT TCC CTA CAC |
| NO: | GAC GCT CTT CCG ATC TTT GGG TTG ATA GTC AGC CTG GTT CC |
| 176. | |
| SEQ ID | AAT GAT ACG GCG ACC ACC GAG ATC TAC ACT CTT TCC CTA CAC |
| NO: | GAC GCT CTT CCG ATC TCC ACA CTT ACT TGG ATT TAT TTT TGT ACT |
| 177. | CAT CCC C |
| SEQ ID | AAT GAT ACG GCG ACC ACC GAG ATC TAC ACT CTT TCC CTA CAC |
| NO: | GAC GCT CTT CCG ATC TTA AAA CAT ACC TGG TCT AAC ACT CAG |
| 178. | AGT TAT TCC |
| SEQ ID | AAT GAT ACG GCG ACC ACC GAG ATC TAC ACT CTT TCC CTA CAC |
| NO: | GAC GCT CTT CCG ATC TTA CAT GGG TTT ACT GTC AGT TTC GTT CC |
| 179. | |
| SEQ ID | AAT GAT ACG GCG ACC ACC GAG ATC TAC ACT CTT TCC CTA CAC |
| NO: | GAC GCT CTT CCG ATC TCC AGG ATT CAC TGT GAG CTG TGT TCC |
| 180 | |
| SEQ ID | AAT GAT ACG GCG ACC ACC GAG ATC TAC ACT CTT TCC CTA CAC |
| NO: | GAC GCT CTT CCG ATC TAG CTT CAC TCT CAC TTG CGT CCC |
| 181. | |
| SEQ ID | AAT GAT ACG GCG ACC ACC GAG ATC TAC ACT CTT TCC CTA CAC |
| NO: | GAC GCT CTT CCG ATC TCC CAG GCT CAC AAT TAA CTC AGT CCC |
| 182. | |
| SEQ ID | AAT GAT ACG GCG ACC ACC GAG ATC TAC ACT CTT TCC CTA CAC |
| NO: | GAC GCT CTT CCG ATC TTA CTT GCT GAG TTT CAT GAT TCC TCT AGT |
| 183. | GTT |
| SEQ ID | AAT GAT ACG GCG ACC ACC GAG ATC TAC ACT CTT TCC CTA CAC |
| NO: | GAC GCT CTT CCG ATC TGT TCC ACA GTC ACA CGG GTT CC |
| 184 | |
| SEQ ID | AAT GAT ACG GCG ACC ACC GAG ATC TAC ACT CTT TCC CTA CAC |
| NO: | GAC GCT CTT CCG ATC TCT GGT TCC ACG ATG AGT TGT GTT CC |
| 185. | |
| SEQ ID | AAT GAT ACG GCG ACC ACC GAG ATC TAC ACT CTT TCC CTA CAC |
| NO: | GAC GCT CTT CCG ATC TGG CTC CAC GAA GAG TTT GAT GCC |
| 186. | |
| SEQ ID | AAT GAT ACG GCG ACC ACC GAG ATC TAC ACT CTT TCC CTA CAC |
| NO: | GAC GCT CTT CCG ATC TCT TAC GTT GTT GTA CCT CCA GAT AGG TTC |
| 187. | C |
| SEQ ID | AAT GAT ACG GCG ACC ACC GAG ATC TAC ACT CTT TCC CTA CAC |
| NO: | GAC GCT CTT CCG ATC TGT CTT ACC TAC AAC TGT GAG TCT GGT GCC |
| 188. | |
| SEQ ID | AAT GAT ACG GCG ACC ACC GAG ATC TAC ACT CTT TCC CTA CAC |
| NO: | GAC GCT CTT CCG ATC TCC TTA CCT ACA ACG GTT AAC CTG GTC CC |
| 189 | |
| SEQ ID | AAT GAT ACG GCG ACC ACC GAG ATC TAC ACT CTT TCC CTA CAC |
| NO: | GAC GCT CTT CCG ATC TCT TAC TCA CCT ACA ACA GTG AGC CAA |
| 190. | CTT CC |
| SEQ ID | AAT GAT ACG GCG ACC ACC GAG ATC TAC ACT CTT TCC CTA CAC |
| NO: | GAC GCT CTT CCG ATC TAT ACC CAA GAC AGA GAG CTG GGT TCC |
| 191. | |
| SEQ ID | AAT GAT ACG GCG ACC ACC GAG ATC TAC ACT CTT TCC CTA CAC |
| NO: | GAC GCT CTT CCG ATC TAA CTT ACC TAG GAT GGA GAG TCG AGT |
| 192. | CCC |
| SEQ ID | AAT GAT ACG GCG ACC ACC GAG ATC TAC ACT CTT TCC CTA CAC |
| NO: | GAC GCT CTT CCG ATC TCT GTC ACA GTG AGC CTG GTC CC |
| 193. | |
| SEQ ID | AAT GAT ACG GCG ACC ACC GAG ATC TAC ACT CTT TCC CTA CAC |
| NO: | GAC GCT CTT CCG ATC TCA CGG TGA GCC GTG TCC C |
| 194. | |
| SEQ ID | AAT GAT ACG GCG ACC ACC GAG ATC TAC ACT CTT TCC CTA CAC |
| NO: | GAC GCT CTT CCG ATC TCC AGT ACG GTC AGC CTA GAG CC |
| 195. | |
| SEQ ID | AAT GAT ACG GCG ACC ACC GAG ATC TAC ACT CTT TCC CTA CAC |
| NO: | GAC GCT CTT CCG ATC TCA CTG TCA GCC GGG TGC C |
| 196. | |
| SEQ ID | AAT GAT ACG GCG ACC ACC GAG ATC TAC ACT CTT TCC CTA CAC |
| NO: | GAC GCT CTT CCG ATC TCA CTG AGA GCC GGG TCC C |
| 197. | |
| SEQ ID | AAT GAT ACG GCG ACC ACC GAG ATC TAC ACT CTT TCC CTA CAC |
| NO: | GAC GCT CTT CCG ATC TAC CAG GAG CCG CGT GCC |
| 198. | |
| SEQ ID | AAT GAT ACG GCG ACC ACC GAG ATC TAC ACT CTT TCC CTA CAC |
| NO: | GAC GCT CTT CCG ATC TCA CGG TCA GCC TGC TGC C |
| 199. | |
| SEQ ID | AAT GAT ACG GCG ACC ACC GAG ATC TAC ACT CTT TCC CTA CAC |
| NO: | GAC GCT CTT CCG ATC TGA CCG TGA GCC TGG TGC C |
| 200. | |
| SEQ ID | AAT GAT ACG GCG ACC ACC GAG ATC TAC ACT CTT TCC CTA CAC |
| NO: | GAC GCT CTT CCG ATC TGT GAA GTT ACT ATG AGC TTA GTC CCT TCA |
| 201. | GCA AA |
| SEQ ID | AAT GAT ACG GCG ACC ACC GAG ATC TAC ACT CTT TCC CTA CAC |
| NO: | GAC GCT CTT CCG ATC TCG AAG TTA CTA TGA GCC TAG TCC CTT TTG |
| 202. | CAA A |
| SEQ ID | AAT GAT ACG GCG ACC ACC GAG ATC TAC ACT CTT TCC CTA CAC |
| NO: | GAC GCT CTT CCG ATC TTG ACA ACA AGT GTT GTT CCA CTG CCA AA |
| 203. | |
| SEQ ID | AAT GAT ACG GCG ACC ACC GAG ATC TAC ACT CTT TCC CTA CAC |
| NO: | GAC GCT CTT CCG ATC TCT GTA ATG ATA AGC TTT GTT CCG GGA |
| 204. | CCA AA |

TABLE 3
exemplary C-segment primers

| Se- | Se- | |
|---|---|---|
| quence | quence | |
| ID | name | Sequence 5′->3′ |
| 205 | IgM | GATGGAGTCGGGAAGGAAGTCCTGT |
| | | GCGAG |
| 206 | IgG | GGGAAGACSGATGGGCCCTTGGTGG |
| 207 | IgA | CAGGCAKGCGAYGACCACGTTCCCATC |
| 208 | IgD | CCACAGGGCTGTTATCCTTT |
| 209 | IgE | AGGGAATGTTTTTGCAGCAG |
| 210 | Igκ | CATCAGATGGCGGGAAGATGAAGACA |
| | | GATGGTGC |
| 211 | Igλ | CCTCAGAGGAGGGTGGGAACAGAGTGAC |
| 212 | TCRB | GCTCAAACACAGCGACCTCGGGTGGGA |
| | | ACAC |
| 213 | TCRA | TCTCTCAGCTGGTACACGGCAGGGT |
| | | CAGGG |

In some embodiments, the composition is a first primer extension primer mixture comprising V-gene specific primers for one or more immune sequence genes. In some embodiments, a first primer extension primer mixture includes primers for one or more of TCR-alpha, TCR-beta, TCR-gamma, TCR-delta and immunoglobulin (IGH, immunoglobulin heavy chain, IGL, immunoglobulin light chain-lambda and IGK, immunoglobulin light chain-kappa). In some embodiments, the mixture comprises multiple primers, e.g., 69 TCR-beta, 8 TCR-delta and 138 IGH V-gene primers.

In some embodiments, the composition is a second primer extension primer mixture comprising J-gene specific primers for one or more immune sequence genes. In some embodiments, a second primer extension primer mixture includes primers for one or more of TCR-alpha, TCR-beta, TCR-gamma, TCR-delta and immunoglobulin (IGH, immunoglobulin heavy chain, IGL, immunoglobulin light chain-lambda and IGK, immunoglobulin light chain-kappa). In some embodiments, the mixture comprises multiple primers, e.g., 14 TCR-beta, 4 TCR-delta and 9 IGH J-gene primers.

In some embodiments, the V-gene primers and J-gene primers for various genes were selected from Table 4.

TABLE 4
V-gene and J-gene primers

| SEQ | | |
|---|---|---|
| ID | | |
| NO: | NAME | SEQUENCE |
| 214 | IGHV3-38 | GGTAGCACATACTACGCAGACTCC |
| 215 | IGHV3-22 | GGACAACAGAATAGACCACGTCTGT |
| 216 | IGHV3OR16-8 | ACAACGCCAATAACTCACCGTATCT |
| 217 | IGHVII-30-21 | GGAGCACAAATTACAACCCACTTCTC |
| 218 | TRBV7-3 | GGGCTGCCCAAAGATCGGT |
| 219 | IGHV3-30 | CAGAGACAATTCCAAGAACACGCTG |
| 220 | IGHV7-34-1 | TGTCAGCACGGCGTGTCTT |
| 221 | TRBV5-3 | AGGGCGCCAGTTCCATGAC |
| 222 | TRBV15 | ACACCCCTGATAACTTCCAATCCAG |
| 223 | IGHV1-58 | CAGTGGTAACACAAACTACGCACAG |
| 224 | TRBV12-1 | CCTGATGTATCATTCTCCACTCTGAGG |
| 225 | IGHV3OR16-6 | GATTCACTTGCAGTAACGCCTGG |
| 226 | IGHV1OR16-1 | GAGGGATCTACTCTGGCAATGGTAAG |
| 227 | TRBV12-3 | CAACAACGTTCCGATAGATGATTCAGG |
| 228 | TRBV30 | TACTCCGTTGGTATTGGCCAGATC |
| 229 | TRBV7-4 | CAGAGTGATGCTCAACGAGACAAATC |
| 230 | IGHV7-56 | TCTGTCAGCACGGCGGATC |
| 231 | TRBV5-6 | TCACCAGTTCCCTAACTATAGCTCTGA |
| 232 | TRBV11-2 | CAGTTGCCTAAGGATCGATTTTCTGC |
| 233 | TRBV1 | CTGACAGCTCTCGCTTATACCTTCAT |
| 234 | TRBV28 | CTCTAGAGAGAAGAAGGAGCGCTTC |
| 235 | TRBV27 | TGAAGGGTACAAAGTCTCTCGAAAAGA |
| 236 | IGHVIII-82 | GCAACAGTGAGTTATCAGGGTTACTCT |
| 237 | IGHVII-28-1 | AATTGTATCTGCTTGCCCCTAGAAGAT |
| 238 | IGHV1OR15-3 | CGCTGGCAATGGTAACACAAAATATTC |
| 239 | IGHV1-24 | GTGAAACAATCTACGCACAGAAGTTCC |
| 240 | IGHV7-4-1 | CACCTCTGTCAGCACGGCA |
| 241 | IGHV3-35 | GTAGGACGCACTATGCAGACTCTG |
| 242 | TRBV6-5 | CCACAGAGGATTTCCCGCTCA |
| 243 | IGHV3-60 | GTGGTGATACCGTACTCTACACAGAC |
| 244 | IGHV4-4 | CCCTCAAGAGTCGAGTCACCATATC |
| 245 | IGHVIII-5-2 | GGGGACAACACTTAACATCACAATCTC |
| 246 | IGHV1OR16-4 | GTGTCCTCCCCACAGGTGC |
| 247 | TRBV10-1 | TTCACGACACTAACAAAGGAGAAGTCT |
| 248 | IGHV1OR16-2 | GTAAGACAGGCTATGCACAGAAGTTTC |
| 249 | IGHV3-43 | GTAGCACATACTATGCAGACTCTGTGA |
| 250 | TRBV5-1 | TCGATTCTCAGGGCGCCAG |
| 251 | TRBV4-1 | CCAAGTCGCTTCTCACCTGAATG |
| 252 | IGHV3-47 | CCATCTCCAGAGACAACGCCA |
| 253 | TRBV3-1 | AGTTCCAAATCGCTTCTCACCTAAATC |
| 254 | IGHV1-2 | TGGCACAAACTATGCACAGAAGTTTC |
| 255 | IGHV3-54 | ATGTTATGCACAATCTGTGAAGAGCAG |
| 256 | TRBV16 | CAAGGAAAGATTTTCAGCTAAGTGCCT |
| 257 | TRBV9 | AACATTCTTGAACGATTCTCCGCAC |
| 258 | IGHV1-67 | CCTGCAAGACTTGTGGATACACCTA |
| 259 | IGHV7-27 | AGTCAAGTGAGACTTCACGCACT |
| 260 | IGHV3-79 | CGAGCAGATTCACCGTCTCCA |
| 261 | IGHV3-63 | AGGACGTTTGTGTATTTTCAGGTGTTC |
| 262 | TRBV24OR9-2 | CGGTTGATCTATTGCTCCTTTGATGTC |
| 263 | IGHVIII-11-1 | TTGTACAGCCCAGCGGTTCA |
| 264 | TRBV7-1 | AGCAGACAAATCGGGGCTTCC |
| 265 | IGHVII-53-1 | CTCCAGATCCATGTCCAAAAAGCAG |
| 266 | TRBVB | CTGAGTCAGCATAGTCATTCCAACCA |
| 267 | IGHV3OR16-10 | AGAGACAATGCCAAGAACTCCTTGTAT |
| 268 | TRBV13 | GATAAAGGAAGCATCCCTGATCGATTC |
| 269 | IGHV1-17 | AATGATAACACACACTACGCACAGAAG |
| 270 | TRBV6-1 | CAATGTCTCCAGATTAAACAAACGGGA |
| 271 | TRBV10-2 | AGTCCCCGATGGCTACGTTG |
| 272 | TRBV23-1 | CACAAGAAGCGATTCTCATCTCAATGC |
| 273 | IGHV2OR16-5 | AATGACAAAAAATCCTACAGCACGTCT |
| 274 | IGHV3OR16-12 | GTAGTAGTGGTTGTAGCACAAACTACG |
| 275 | IGHV1-45 | GTAACACCAACTACGCACAGAAATTCC |
| 276 | IGHV3-49 | TACGCCGCGTCTGTGAAAGG |
| 277 | IGHVII-43-1 | CTCCAGATCTATGTCCAAAAACAGCTC |
| 278 | IGHV3-76 | GGTACAGCTTGGGGGGTCC |
| 279 | IGHVII-51-2 | GCATAGGTCACGAGGGAGCA |
| 280 | IGHV5-51 | AGTCCATCAGCACCGCCTAC |
| 281 | IGHVIII-26-1 | TGGTTTCGGGTTTACTGGGTGC |
| 282 | TRBV6-7 | AATGTCTCCAGATCAAACACAGAGGAT |
| 283 | TRBV7-6 | GGCTGCCCAATGATCGGTTC |
| 284 | IGHV3-42 | CTCATTTGCAGCTTCTAGATTCACCTT |
| 285 | TRBV25OR9-2 | CTCCAGAATAAGGATAGAGCGTTTTCC |
| 286 | TRBV8-2 | GAACAGTGTTCTGATATCGACAAGACC |
| 287 | TRBV5-2 | GGAAACTTGCCTAATTGATTCTCAGCT |
| 288 | IGHVII-44-2 | TCCTTCTCCAGAGATTCATCCAAGAAA |
| 289 | TRBV25-1 | GTCTCCAGAATAAGGACGGAGCATTT |
| 290 | IGHV8-51-1 | ATATGGCGTGGTGAAAGTCATCAATAC |
| 291 | TRBV7-2 | GGCTGCCCAGTGATCGCTT |
| 292 | TRBV22-1 | GAGATCTAACTGAAGGCTACGTGTCT |
| 293 | IGHV1OR15-6 | TTGAGTGGATGGAACGTGTTGATCC |
| 294 | IGHV1-68 | TGGTAACACCAACTATGCAAAGAAGTT |
| 295 | IGHVII-62-1 | CATAGGTCATGAAGGGAGCACACATTA |
| 296 | IGHV3OR16-16 | GTGACAGAACAGTGGCTATGTGTG |
| 297 | IGHV3-50 | CCTGAGACTCTGCCGTGCA |
| 298 | IGHV7-81 | CAGGGCTTCACAGGACGGT |
| 299 | IGHV3-41 | CTCCAGAGACAATTCTAAGAGCATGCT |
| 300 | IGHV3-29 | GCCGAGTTCACCAGTCTCCA |
| 301 | TRBV20-1 | CCACATACGAGCAAGGCGTC |
| 302 | TRBV18 | GCCAAAGGAACGATTTTCTGCTGAAT |
| 303 | IGHV3-25 | GCAAAGCCTGCGTGGTCC |
| 304 | IGHV3-15 | CGCTGCACCCGTGAAAGG |
| 305 | TRBV17 | CAGTACCAAAACATTGCAGTTGATTCA |
| 306 | IGHVII-74-1 | ACTACATCACAAACAGTGCTTATGACT |
| 307 | TRBV19 | AGAAAGGAGATATAGCTGAAGGGTACA |
| 308 | IGHV7-40 | ACCAACGGCTTCACAGGACG |
| 309 | IGHVII-22-1 | TGCTTCTCCATTACAACCAGTGCTT |
| 310 | TRBV24-1 | AAAGGAGAGATCTCTGATGGATACAGT |
| 311 | IGHV3-16 | CTCCGTGAAGCGCCGATTC |
| 312 | TRBV5-4 | TCTCCAGTTCCCTAATTATAGCTCTGA |
| 313 | IGHVII-33-1 | CATCTCCAGATCCATGTCCAAAAAGTA |
| 314 | IGHV4-80 | GGTGCAGCTGCAGGAGTGG |
| 315 | IGHV1OR15-4 | GGTAACACAACATATGCACAGAAGTTC |
| 316 | TRBV14 | CAACAATCGATTCTTAGCTGAAAGGAC |
| 317 | TRBV5-5 | AGCTCGCCAGTTCCCTAACTATAG |
| 318 | IGHV3OR16-11 | CACATACTATGCAGACTCCGTGAAG |
| 319 | IGHV3OR15-7 | AGCTAACAGTTACACGACAGAATATGC |
| 320 | IGHVIII-13-1 | GTCAGACAGAGAAATACTACAGACCAG |
| 321 | IGHVIII-38-1 | ACTCGCCTTCAGTACAAAGAAGATTAA |
| 322 | IGHV3-57 | AAGTGGGAGTTCTCAGAGTTACTCTCC |
| 323 | IGHV1-69-2 | GTCACCATAACCGCGGACAC |
| 324 | IGHV1-14 | AGGGACACGTCCACGAGC |
| 325 | TRBV29-1 | CCAAACCTAACATTCTCAACTCTGACT |
| 326 | IGHVII-65-1 | CATCCATCACCCCCCGCA |
| 327 | IGHV3-32 | GATGATGGAAGTCAGATACACCATGCA |
| 328 | IGHV3-6 | GGGTCCCAGTTATTAGTGGTAGTGGTA |
| 329 | IGHV3-52 | AAAGTGTGACGGAAGTGAGAAATACTA |
| 330 | IGHVIII-2-1 | GAGTGATCAAGTATGAATTCTCAGGGT |
| 331 | TRBV12-2 | CCTGATGGATCATTCTCTACTCTGAAG |
| 332 | IGHV3-37 | GAATGGGTCTCATACATTAGTGCTAGT |
| 333 | TRBV20OR9-2 | GGCTCCGAGGTCACATACGA |
| 334 | IGHV4-55 | TCGAATCACCATGTCCGTAGACA |
| 335 | IGHVII-49-1 | TGCTTCTCCATCACAACCAGTG |
| 336 | TRBV6-8 | GCTGCTGGTACTACTGACAAAGAAGTC |
| 337 | TRBV7-5 | CCTTCCAGGATGAAACTCAACAAGATA |
| 338 | IGHV1-69D | CACGATTACCGCGGACGAAT |
| 339 | TRBV26OR9-2 | GAGATGTCTCTGAGAGGTATCATGTTT |
| 340 | IGHV3OR16-7 | CTGGAGTGGGTTGGCCGTAT |
| 341 | IGHV2-5 | ATTTATTGGGATGATGATAAGCGCTAC |
| 342 | TRBV21-1 | AGAAAGCAGAAATAATCAATGAGCGAT |
| 343 | TRBV2 | GCCTGATGGATCAAATTTCACTCTGA |
| 344 | IGHV3-30-2 | ACAGTGTGATGGAAGTCAGATATGTTA |
| 345 | TRBV22OR9-2 | CTGAGACTGATCTATTACTCAAGGGTT |
| 346 | TRBV26 | CCCTGTCTCTATTTGATCATCCATTTT |
| 347 | TRBVA | GCCGACTCATTATTCAGTTAACATTGA |
| 348 | TRBV6-2 | GATGGCTACAATGTCTCCAGATTAAAA |
| 349 | TRBV6-4 | CCATTATTCAAATACTGCAGGTACCAC |
| 350 | TRBV7-7 | CAATTATGAAGCTCAACCAGACAAATC |
| 351 | TRBV7-9 | CTTACTTCCAGAATGAAGCTCAACTAG |
| 352 | TRBV10-3 | TGGTGTTAAAGATACTGACAAAGGAGA |
| 353 | IGHV1OR15-2 | ACACTTACAATGGTAACACAAACTACC |
| 354 | IGHV3-36 | TCATTTATGAGTTGTTGTGTAGGTAGC |
| 355 | IGHV3-75 | GTCTCATGTATTAGTACTGATGGGAGT |
| 356 | IGHVII-1-1 | GCCCTCTGGGAAGGCGCT |
| 357 | IGHVII-15-1 | GGATTTCCAATCATAACCAGTACTTCC |
| 358 | IGHVIII-44 | ACACTTTACAGACACCATCAATTTTCC |
| 359 | IGHVIII-76-1 | ACCCTCCATCAATACAAAGAAAAATCA |
| 360 | IGHVIV-44-1 | GTGATATGGGTTAAGGGAAACACTAAG |
| 361 | IGHV1-18 | CGCTTACAATGGTAACACAAACTATGC |
| 362 | IGHV2-70 | TGGGATGATGATAAATACTACAGCACA |
| 363 | IGHV2-70D | TTGGGATGATGATAAATTCTACAGCAC |
| 364 | IGHV4-28 | CTATTATAGTGGGAGCACCTACTACAA |
| 365 | IGHV6-1 | GTCCAAGTGGTATAATGATTATGCAGT |
| 366 | IGHVII-26-2 | TAGACTGGATCATCAAGGAATACACAT |
| 367 | IGHV3OR16-13 | GCACAAGCTACGCAGACTCC |
| 368 | IGHV3-13 | GGTGACCCATACTATCCAGGCT |
| 369 | TRBV8-1 | AGCATGACCAAAGGCGGTG |
| 370 | TRBV12-5 | GCAACCGGGCTCCTCTAGA |
| 371 | IGHV3-73 | ATGCTGCGTCGGTGAAAGG |
| 372 | IGHV5-78 | CCTTCCAAGGCCACGTCA |
| 373 | IGHV1OR15-9 | AACCAGGGACACATCCATGG |
| 374 | IGHV1OR21-1 | TAGTGATGGCAGCACAAGCT |
| 375 | IGHV1-69 | CACGATTACCGCGGACAAATC |
| 376 | IGHV3OR16-9 | GTGGTTACACAAACTACGCAGAC |
| 377 | IGHV3OR16-15 | CGGTAAGACGCACTATGTGGA |
| 378 | IGHV2-26 | TCGAATGACGAAAAATCCTACAGC |
| 379 | IGHJ3 | GTCACCGTCTCTTCAGGTAAGATGG |
| 380 | TRBJ1-6 | CTCTTGACTCGGGGGTGCC |
| 381 | TRBJ2-4 | GCTCGGGTTTTTGTGCGGG |
| 382 | TRBJ1-3 | TGTTGTAGGTGAGTAAGTCAAGGCTG |
| 383 | IGHJ2 | GAGTCCCACTGCAGCCCC |
| 384 | IGHJ6 | CCTCAGGTAAGAATGGCCACTCTAG |
| 385 | TRBJ2-3 | CGTCGCAGGGCCAGTTTCT |
| 386 | TRBJ2-7 | AGTCGGAGGGTGGACCGG |
| 387 | TRBJ1-5 | CTCTCCATCCTAGGTAAGTTGCAGAAT |
| 388 | IGHJ1 | GGTCACCGTCTCCTCAGGTG |
| 389 | TRBJ2-6 | GTGAGTTTTCGCGGGACCAC |
| 390 | IGHJ3P | TACGTGGGAGGCCAGCAGA |
| 391 | TRBJ1-2 | GTTAACCGTTGTAGGTAAGGCTGG |
| 392 | TRBJ2-1 | CACGACCCCAGAACCCTGT |
| 393 | TRBJ1-4 | GATAGTGTATCATAAGGTCGGAGTTCC |
| 394 | TRBJ2-2 | TGGGTAAGGAGGCGGTTGG |
| 395 | TRBJ2-5 | TTGGGTCTGGTTTTTGCGGG |
| 396 | TRBJ1-1 | TGTCCCTTTTAGAGTGGCTATATTCTT |
| 397 | IGHJ2P | GGTCTCAGCCCGGGGGTC |
| 398 | IGHJ1P | CCTACCAGCCGCAGGGTT |
| 399 | TRBJ2-2P | GCACCGGTTTTTGTCCTGG |

In some embodiments, the composition is a second primer extension mixture comprising J-gene specific primers and further comprising an opposite-facing primer capable of hybridizing to a primer binding site (universal primer binding site) in the first primer extension primer. In some embodiments, the opposite-facing primer is a shortened sequencing primer. In some embodiments, opposite-facing primer is a sequencing primer to be used in a subsequencing sequencing step, the primer being shortened by removing the index sequence.

Described herein are target polynucleotides and compositions containing such target polynucleotides. In some cases, a composition containing such target polynucleotides further contains a DNA-dependent polymerase and/or a RNA-dependent DNA polymerase, a ligase, a first primer or plurality of first primers, a second primer or plurality of second primers, first or second primer polymerase extension products, or a combination thereof. Generally, the target polynucleotides encode immune cell receptors, such as B cell receptors (i.e., antibodies), or T cell receptors, a complement thereof, or portions thereof. The target polynucleotides can be single stranded or double-stranded. The target polynucleotides can be DNA (e.g., genomic DNA). The target polynucleotides can be RNA (e.g., mRNA). The target polynucleotides can be cDNA generated by reverse transcription of mRNA. In an exemplary embodiment, the target polynucleotides are genomic DNA or cDNA that is heat denatured to form single-stranded targets.

In some embodiments, the target polynucleotides are obtained from a sample enriched for immune cells. For example, the target polynucleotides can be obtained from a sample enriched for T cells, enriched for B cells, enriched for T cells and B cells, enriched for lymphocytes, or enriched for peripheral blood mononuclear cells (PBMCs). In some cases, the target polynucleotides are obtained from a sample enriched for a fraction of T cells or B cells. For example, the sample can be enriched for T cells that express α/β TCRs. As another example, the sample can be enriched for T cells that express γ/δ TCRs. As yet another example, the sample can be enriched for B cells that express a certain isotype of BCR, or a set of such isotypes, such as IgA, IgG, IgM, IgE, or a combination thereof. As yet another example, the sample can be enriched for B cells expressing kappa light chain BCRs. As yet another example, the sample can be enriched for B cells expressing lambda light chain BCRs. As yet another example, the sample can be enriched for T cells that express α/β TCRs or γ/δ TCRs, wherein the sample is obtained by flow cytometry. As yet another example, the sample can be a FFPE tissue sample containing infiltrating lymphocytes. As yet another example, the sample can be a FFPE tissue sample, wherein the sample contains, or is suspected of containing, one or more tumor cells. Methods for enriching sample for a specific immune cell type include, but are not limited to, methods employing one or more of the following: ultracentrifugation, FICOLL™ gradient centrifugation, or flow cytometry (e.g., fluorescence-activated cell sorting (FACS)).

Described herein are reaction mixtures that contain target polynucleotides (e.g., target polynucleotides encoding B or T cell receptors, a complement thereof, or portions thereof), first primers, first primer extension products (e.g., single-stranded or hybridized to a target polynucleotide), second primers, second primer extension products (e.g., single-stranded or hybridized to a target polynucleotide), adapters (e.g., double stranded adapters, such as splint adapters), or a combination thereof. In some cases, the reaction mixture further contains a DNA-dependent DNA polymerase and/or a RNA-dependent DNA polymerase and reagents for polymerase-mediated and template-directed primer extension (e.g., divalent cations such as magnesium cations, nucleotide triphosphates, buffers, salts, etc.). In some cases, the DNA polymerase exhibits strand-displacing activity. In some cases, the DNA polymerase exhibits exonuclease activity. In some cases, the DNA polymerase does not exhibit or does not exhibit substantial strand-displacing activity. In some cases, the DNA polymerase does not exhibit or does not exhibit substantial exonuclease activity. In some cases, the DNA polymerase is thermostable. In some cases, the reaction mixture contains DNA ligase. In some embodiments, the RNA-dependent DNA polymerase is a telomerase. In some embodiments the RNA-dependent DNA polymerase lacks 3′-5′ exonuclease activity. In some embodiments, the RNA-dependent DNA polymerase is thermostable.

In some embodiments, the DNA polymerase has long-range capability. In some embodiments, the DNA polymerase has proofreading capability including processing proofreading capability. In some embodiments, the DNA polymerase has high fidelity. In some embodiments, the DNA polymerase is a mixture including Taq polymerase and an engineered archaeal B-family polymerase. In some embodiments, the polymerase has hot-start capability. In some embodiments, the hot-start capability is conferred by a thermolabile polymerase-specific antibody.

In some embodiments, the reaction mixture comprises an exonuclease. In some embodiments, the exonuclease is a single-strand exonuclease. In some embodiments, the exonuclease is thermolabile. In some embodiments, the exonuclease is inactivated at or below 95° C. In some embodiments, the exonuclease is inactivated at 80° C.

In some embodiments, the reaction mixture contains a plurality of structurally different target polynucleotides, wherein individual target polynucleotides of the plurality each comprise immune cell receptor V gene regions, optionally D gene regions, optionally C gene regions, and J gene regions (e.g., target polynucleotides encoding immune cell receptor VJ, VDJ, or VJ and VDJ regions); and a plurality of first primers having [FW] regions. In some embodiments, the reaction mixture contains a plurality of structurally different target polynucleotides, wherein individual target polynucleotides of the plurality each comprise immune cell receptor V gene regions, optionally D gene regions, optionally C gene regions, and J gene regions (e.g., immune cell receptor VJ, VDJ, or VJ and VDJ regions); and a plurality of first primers having [J] regions or [C] regions.

In some embodiments, the first primers of the reaction mixture are hybridized to the target polynucleotides of the reaction mixture. In some embodiments, the reaction mixture contains a plurality of structurally different target polynucleotides hybridized to a plurality of first primer extension products (i.e., products of a DNA-polymerase-mediated and template-directed extension reaction). In some embodiments, the reaction mixture contains a plurality of structurally different immune cell receptor encoding target polynucleotides hybridized to a plurality of first primer DNA polymerase extension products.

In some embodiments, the reaction mixture contains a plurality of single-stranded target polynucleotides, the individual target polynucleotides each comprising the following from regions from 5′ to 3′: a sequencer-specific adapter sequence, optionally a multiplex identifier (MID) barcode, a unique molecular identifier (UID) barcode, at least a portion of an immune cell receptor framework 3 region, an immune cell receptor CDR3 region, an optional immune cell receptor diversity (D) region, an optional immune cell receptor constant (C) region, and at least a portion of an immune cell receptor J region, or the complements thereof. In some cases, such a plurality of single-stranded target polynucleotides are first primer extension products. In some cases, such a plurality of single-stranded target polynucleotides are first primer extension products ligated to an adapter comprising a universal primer binding site and optionally an MID barcode.

In some embodiments, the reaction mixture contains a plurality of single-stranded target polynucleotides, the individual target polynucleotides each comprising the following from regions from 5′ to 3′: a sequencer-specific adapter sequence, optionally a multiplex identifier (MID) barcode, a unique molecular identifier (UID) barcode, at least a portion of an immune cell receptor J-gene region, an optional D region, an optional immune cell receptor constant (C) region, an immune cell receptor CDR3 region, and at least a portion of an immune cell receptor framework 3 region, or the complements thereof. In some cases, such a plurality of single-stranded target polynucleotides are first primer extension products. In some cases, such a plurality of single-stranded target polynucleotides are first primer extension products ligated to an adapter comprising a universal primer binding site and optionally an MID barcode.

In some embodiments, a reaction mixture can contain one or more of the foregoing first primer extension products (e.g., adapter ligated first primer extension products) hybridized to a plurality of second primer extension products. In cases where the first primers hybridize to a framework region of an immune cell receptor encoding target polynucleotide, the second primers can be configured to hybridize to a J gene region or C gene region of the first primer extension products. In cases, where first primers hybridize to a complement of a J region or C region of an immune cell receptor encoding target polynucleotide, the second primers can be configured to hybridize to a framework region of the first primer extension products.

Described herein are adapters for downstream amplification or sequencing applications. Attachment of an adapter to one or both ends of a target polynucleotide, first primer extension product, or second primer extension product, can attach a UID barcode, an MID barcode, a universal primer binding site or complement thereof, or a combination thereof. Adapters containing a universal primer binding site or complement thereof can be referred to as universal adapters. In some embodiments, adapters are attached by ligation. In some embodiments, adapters are attached by hybridizing a primer containing an adapter sequence (e.g., an adapter sequence comprising or consisting of a universal primer binding site or complement thereof) to a target polynucleotide, first primer extension product, or second primer extension product. In some embodiments, the primer containing an adapter sequence is a second primer that can be hybridized to a first primer extension product and extended with a polymerase as described herein. In some embodiments, first adapters are ligated to a first primer extension product and second adapters are attached by hybridization of a second primer containing such an adapter to the first primer extension product and extending the second primer.

In some embodiments, one or more adapters are splint adapters. Splint adapters can be hybridized to a [SPLINT] region of a primer extension product (e.g., a first primer extension product) and ligated to a [5′-Phos] of the first primer extension product. Splint adapters can contain a double stranded region and a 5′ single-stranded overhang region, wherein the 5′ single-stranded overhang region is complementary to and hybridizes under hybridization conditions with the [SPLINT] region of the primer extension product (e.g., a first primer extension product). The 5′ single-stranded overhang region can be at least 2 nucleotides in length, at least 4 nucleotides in length, at least 6 nucleotides in length, at least 8 nucleotides in length, from 2 to 10 nucleotides in length, or from 2 to 8 nucleotides in length. In some embodiments, the 5′ single-stranded overhang region comprises or consists of 6 consecutive nucleotides that are complementary to the [SPLINT] region of the first primer extension product. In some embodiments, the 5′ single-stranded overhang region comprises or consists of the sequence AGA TCG.

Splint adapters can contain a barcode and a universal primer binding site as described herein. In some cases, the splint adapter contains a MID barcode. In some cases, the splint adapter contains an MID barcode and a universal primer binding site. In some cases, the barcode is encoded in the double-stranded region of the splint adapter. In some cases, the universal primer binding site is encoded in the double-stranded region of the splint adapter. In some cases, the barcode (e.g., MID barcode) and the universal primer binding site is encoded in the double-stranded region of the splint adapter. In some embodiments, with respect to the strand of the splint adapter that contains the 5′ single-stranded overhang region, in some cases, the universal primer binding site can be 3′ of the barcode (e.g., MID barcode).

In some embodiments, the reaction mixture contains universal amplification primers hybridizing to adapter sequences or universal primer binding sites in first and second round primer extension primers. The universal amplification primers may comprise sequencing platform-specific sequences. Based on the platform, the sequencing primers may include barcode (index) sequences, e.g., sample index sequences. One or both sequencing primers may comprise index sequences.

In some embodiments, prior to universal amplification with universal primers, excess primers from the second round of primer extension is removed by an exonuclease. In some embodiments, the exonuclease is added to a reaction mixture comprising excess extension primers to be degraded and also comprising universal amplification primers to be retained. In some embodiments, universal amplification primers comprise one or more modifications conferring exonuclease resistance.

In some embodiments, the nuclease-protecting modification allows to advantageously skip a purification step between the second primer extension and universal amplification thus saving time, resources and reducing the possibility of sample contamination.

In some embodiments, the exonuclease is a 3′-5′-exonuclease and the modification is selected from one or more nucleotides at the 3′-end modified with one of the following: a phosphorothioate (PS) bond, a 2′-O-methyl (2′OMe), a 2′-fluoride and Inverted ddT. In some embodiments, the modification is a nucleotide with a phosphorothioate (PS) bond, which substitutes a sulfur atom for a non-bridging oxygen in the phosphate backbone of an oligonucleotide. In some embodiments, the modification is a nucleotide with 2′-O-methyl (2′OMe) modification of the ribose/deoxyribose yielding 5- to 10-fold reduced susceptibility to DNases. In some embodiments, the modification is a nucleotide with 2′-fluoro modification. The fluorine-modified ribose/deoxyribose confers some relative nuclease resistance. In some embodiments, the modification is a 2′,3′ dideoxy-dT base (5′ Inverted ddT) at the 3′ end of an oligonucleotide protecting against some forms of enzymatic degradation. In some embodiments, the universal primer has more than one modified nucleotide and more than one type of modified nucleotide.

In some embodiments, the methods described herein can utilize a plurality of immune cell receptor V gene specific first primers each comprising an [FW] region at a 3′ end for hybridizing to a framework (e.g., framework 1, framework 2, or framework 3) region of a target polynucleotide encoding an immune cell receptor. In such embodiments, the plurality of second primers each comprise a [J] region to flank the region of interest. Alternatively, the methods can utilize a plurality of immune cell receptor J gene specific first primers each comprising a [J] region at a 3′ end for hybridizing to a J region of a target polynucleotide encoding an immune cell receptor. In such embodiments, the plurality of second primers each comprise a [FW] region to such that the first and second primers flank the region of interest in the target polynucleotides. In another embodiment, the methods can utilize a plurality of immune cell receptor C gene specific first primers each comprising a C-segment region at a 3′ end for hybridizing to a C gene region of a target polynucleotide encoding an immune cell receptor. In such embodiments, the plurality of second primers can each comprise a [V] gene region such that the first and second primers flank the region of interest in the target polynucleotides.

In some embodiments, the method includes: a) providing a reaction mixture containing: i) a plurality of structurally different target polynucleotides as described herein, wherein the individual target polynucleotides encode immune cell receptor V gene regions, optionally D gene regions, optionally C gene regions, and J gene regions; and, ii) a plurality of immune cell receptor V gene specific primers or C gene specific primers (i.e., first primers) as described herein, wherein the immune cell receptor V gene specific primers are hybridized to the V gene regions of the target polynucleotides or wherein the immune cell receptor C gene specific primers are hybridized to the C gene regions of the target polynucleotides; b), extending the hybridized immune cell receptor V gene specific primers or C gene specific primers with a polymerase to generate extended immune cell receptor V gene specific primers or extended immune cell receptor C gene specific primers (i.e., first primer extension products) and then removing un-extended immune cell receptor V or C gene specific primers, if present, thereby generating extended immune cell receptor V gene specific primers containing at least a portion of the immune cell receptor V region, optionally the immune cell receptor D region, and at least a portion of the immune cell receptor J region or at least a portion of the immune cell receptor C region, optionally the immune cell receptor D region, and at least a portion of the immune cell receptor V region. In some cases, the removing un-extended immune cell receptor V or C gene specific primers includes contacting the un-extended immune cell receptor V or C gene specific primers with a single-stranded DNA exonuclease enzyme and thereby digesting the un-extended immune cell receptor V or C gene specific primers.

In some embodiments, a method comprises the following steps: DNA extraction, optional DNA quality assessment, first round of primer extension, thermolabile exonuclease treatment, purification, second round of primer extension and optional purification thereby forming a library of enriched nucleic acid sequences. Depending on the downstream use of the enriched library, the method may further comprise one or more of the following: library amplification, purification, quality control assessment, pooling of libraries and sequencing of the libraries or pools.

In some embodiments, the first primer extension step comprises two or more repetitions of the annealing step. In some embodiments, the annealing step comprises a temperature profile including several (e.g., 2, 3 or more) progressively lower annealing temperatures. Methods utilizing a step-wise annealing procedure increases specificity of the target enrichment method. In some embodiments, utilizing repetitions of the step-wise annealing procedure further increases specificity of the target enrichment method. Disclosed herein is an improved primer extension target enrichment method wherein the annealing temperature profile comprises one or more of a series of decreasing temperatures. In some embodiments, the annealing step comprises 2, 3, 4, 5, 6, 7, 8, 9, 10, 20 or more rounds of thermocycling through the series of two or more annealing temperatures. In some embodiments, by way of example, the annealing step comprises 20 cycles wherein each cycle consists of an incubation at 60° C., at 57.5° C. and at 55° C.

In some embodiments, the first primer extension step comprises a round of denaturation, two or more rounds of step-wise annealing, and a round of extension.

In some embodiments, the first primer extension step utilizes the following temperature profile: [initial denaturation]−[two or more rounds of the annealing temperature profile]−[extension]. In some embodiments, by way of example, the first primer extension step utilizes the following temperature profile:

| Stage | Temp (° C.) | Duration | | Cycles |
|---|---|---|---|---|
| Initial Denaturation | 95° C. | 10 | min | 1 |
| Intermediate Temp | 80° C. | 0 | sec | |
| Annealing (ramp at 0.2° C./sec) | 60° C. | 0 | sec | |
| Annealing high | 60° C. | 20 | sec | 20 |
| Annealing mid | 57.5° C. | 20 | sec | |
| Annealing low | 55° C. | 20 | sec | |
| Extension | 72° C. | 2 | min | 1 |

| | | | |
|---|---|---|---|
| End | 4° C. | ∞ | 1 |

In some cases, the removing un-extended immune cell receptor V or C gene specific primers includes solid phase reversible immobilization of single-stranded first primer extension products or double stranded polynucleotides containing single-stranded target polynucleotide hybridized to single-stranded first primer extension product. In some cases, the removing un-extended immune cell receptor V or C gene specific primers includes resin (e.g., silica (e.g., silica bead)) or membrane-based column or batch purification of double-stranded DNA from single-stranded DNA or purification of a selected size of single or double stranded DNA from the reaction mixture. In some cases, the removing includes removing single or double-stranded DNA, or a combination thereof, having a size of less than about 100 bases or base pairs. In some cases, the removing includes removing single or double-stranded DNA, or a combination thereof, having a size of more than about 1,000 bases or base pairs. In some cases, the removing includes purifying from the sample primer extension products or double-stranded DNA containing primer extension products, or a combination thereof, having a size of more than about 100 bases or base pairs and less than about 1,000 bases or base pairs. In some cases, the removing further removes genomic DNA or denatured (e.g., single-stranded) genomic DNA from the sample. In some embodiments, the removing further removes denatured cDNA from the sample.

In some embodiments, the method includes: c) hybridizing a first universal adapter (e.g., a splint adapter containing a universal primer binding site) to a [SPLINT] adapter hybridization site of the extended immune cell receptor V or C gene specific primers; d) ligating the hybridized first universal adapter to the extended immune cell receptor V or C gene specific primers, and then removing un-ligated adapters, if present. In some cases, the removing un-ligated adapters includes solid phase reversible immobilization of adapter-ligated single-stranded first primer extension products or double stranded polynucleotides containing single-stranded target polynucleotide hybridized to such adapter-ligated single-stranded first primer extension product. In some cases, the removing un-ligated adapters includes resin (e.g., silica (e.g., silica bead)) or membrane-based column or batch purification of double-stranded DNA from single-stranded DNA or purification of a selected size of single or double stranded DNA from the reaction mixture. In some cases, the removing includes removing from a reaction mixture single or double-stranded DNA, or a combination thereof, having a size of less than about 100 bases or base pairs. In some cases, the removing includes removing from a reaction mixture single or double-stranded DNA, or a combination thereof, having a size of more than about 1,000 bases or base pairs. In some cases, the removing includes purifying from the sample adapter-ligated primer extension products or double-stranded DNA containing such adapter-ligated primer extension products, or a combination thereof, having a size of more than about 100 bases or base pairs and less than about 1,000 bases or base pairs.

In some embodiments, removing the un-extended immune cell receptor V or C gene specific primers includes contacting the sample with an exonuclease. In some embodiments, the exonuclease has a single-strand degrading activity and lacks the double-strand degrading activity. In some embodiments, the exonuclease is thermolabile. In some embodiments, the exonuclease is Exonuclease I.

In some embodiments, the nuclease-protecting modification of universal amplification primers used in the amplification step allows to advantageously skip a purification step between the second primer extension and universal amplification thus saving time, resources and reducing the possibility of sample contamination.

In some embodiments, the method includes: e), hybridizing a plurality of immune cell receptor J gene specific primers (i.e., second primers) to the J region portions of the extended immune cell receptor V gene specific primers (i.e., first primer extension products), wherein the immune cell receptor J gene specific primers comprise a 3′ J gene hybridizing region and a 5′ second universal adapter region or hybridizing a plurality of immune cell receptor V gene specific primers (i.e., second primers) to the V region portions of the extended immune cell receptor C gene specific primers (i.e., first primer extension products), wherein the immune cell receptor V gene specific primers comprise a 3′ V gene hybridizing region and a 5′ second universal adapter region; and, f) extending the hybridized immune cell receptor J gene specific primers or extending the immune cell receptor V gene specific primers with a polymerase, thereby forming a plurality of structurally different double-stranded products comprising an extended immune cell receptor J gene specific primer or extended immune cell receptor V gene specific primer (i.e., second primer extension product) hybridized to an (e.g., adapter-ligated) extended immune cell receptor V gene specific primer or extended immune cell receptor C gene specific primer, each double-stranded product comprising at least a portion of the immune cell receptor V region, optionally the immune cell receptor D region, and at least a portion of the immune cell receptor J region flanked by a first and second universal adapter sequence or at least a portion of the immune cell receptor C region, optionally the immune cell receptor D region, and at least a portion of the immune cell receptor V region flanked by a first and second universal adapter sequence.

In some embodiments, the e) hybridizing and f) extending can be repeated multiple times by heating to denature double-stranded products produce in the extending of f) (e.g., double-stranded DNA products comprising first primer extension products (e.g., adapter-ligated extended immune cell receptor V or C gene specific primer) hybridized to second primer extension products (e.g., extended immune cell receptor J or V gene specific primer)), cooling to hybridize un-extended second primers to first primer extension products (e.g., adapter-ligated first primer extension products), and extending hybridized primers. In some cases, e) and f) are repeated 1, 2, 3, 4, 5, 6, 7, 8, 9, 10, 11, 12, 13, 14, 15 or more times. In some cases, e) and f) are repeated from 2 to 15 times, from 3 to 12 times, from 5 to 10 times, or from 5 to 15 times.

In some embodiments, after extension of hybridized second primers, a polynucleotide containing at least a portion of a J-region, an optional D region, an optional C region, and at least a portion of a V region, flanked by universal primer binding sites, or a complement thereof is provided. This polynucleotide can be amplified by universal PCR with an amplification reaction mixture containing the polynucleotide, a forward universal primer and a reverse universal primer.

In some embodiments, extension of the second primer is performed in the presence of the opposite-facing primer hybridizing to the primer-binding site introduced into the primer extension product by the first primer.

In some embodiments, the second primer extension step comprises two or more repetitions of the annealing step. In some embodiments, the annealing step comprises a temperature profile including several (e.g., 2, 3 or more) annealing temperatures. In some embodiments, the annealing temperature profile comprises a series of decreasing temperatures. In some embodiments, the annealing step comprises 2, 3, 4, 5, 6, 7, 8, 9, 10, 20 or more rounds of thermocycling through the series of two or more annealing temperatures. In some embodiments, by way of example, the annealing step comprises 20 cycles wherein each cycle consists of an incubation at 60° C., at 57.5° C. and at 55° C.

In some embodiments, the second primer extension step comprises a round of initial denaturation, two or more rounds of: denaturation, step-wise annealing and extension, and a round of final extension. In some embodiments, by way of example, the second primer extension step comprises 10 cycles.

In some embodiments, the second primer extension step utilizes the following temperature profile: [initial denaturation]−[two or more rounds of: denaturation, annealing temperature profile, extension]−[final extension]. By way of example, the following thermal profile may be used.

| Stage | Temp (° C.) | Duration | | Cycles |
|---|---|---|---|---|
| Initial Denaturation | 95° C. | 5 | min | 1 |
| Denature | 95° C. | 30 | sec | 10 |
| Annealing high | 60° C. | 10 | sec | |
| Annealing mid | 57.5° C. | 10 | sec | |
| Annealing low | 55° C. | 10 | sec | |
| Extension | 72° C. | 45 | sec | |
| Final Extension | 72° C. | 1 | min | 1 |

| | | | |
|---|---|---|---|
| End | 4° C. | ∞ | 1 |

In some embodiments, the method further comprises contamination removal or reducing the amount of contaminating sequences in the sequencing data output. As disclosed herein, the contamination filtering process is a novel combination of physical steps and analytical steps. In some embodiments, during amplification, a library may become contaminated by a nucleic acid fragment also containing universal amplification primer binding sites. For example, a fragment from another library or a pool of libraries may contaminate a reaction mixture described herein. In one embodiment, the primer extension target enrichment method disclosed herein includes a method of detecting and removing from sequencing data output the sequencing data originating from contaminating nucleic acids, the method comprising: 1) an increased number of primer extension cycles in the first round of primer extension; alone or in combination with 2) one or more rounds of exponential amplification in the second round of primer extension. In some embodiments, these extra rounds increase the ratio of true targets to contaminants in the sample subjected to sequencing and facilitate detection and removing of the sequencing data originating from the contaminants from the sequencing data output. The additional rounds of extension and added pre-amplification steps will increase the proportion of UMI (unique molecular index) family sizes of true targets in a mixture comprising true targets and contaminants. The proportions of molecule counts (by UMI sequences) are maintained by PCR, and sequencing. Therefore at the completion of sequencing, the contaminant reads appear as a much smaller population within the sequencing data output. In some embodiments, a bi-modal UMI family size distribution is obtained.

In some embodiments, the method further comprises after sequencing, a step of calculating the number of nucleic acid sequences belonging to a UMI family (a group of nucleic acid sequences sharing an identical UMI). In some embodiments, the method further comprises identifying and retaining in the sequencing data output the top 90% of UMI families by size. In some embodiments, the method further comprises identifying and retaining in the sequencing data output the top 91%, 92%, 93%, 94%, 95% 96%, 97%, 98% or 99% of UMI families by size. In some embodiments, the method further comprises removing from the sequencing data output the bottom 10% of UMI families by size. In some embodiments, the method further comprises removing from the sequencing data output the bottom 9%, 8%, 7%, 6%, 5%, 4%, 3%, 2% or 1% of UMI families by size.

In some embodiments, the number of primer extension cycles in the first round of primer extension and the number of primer extension cycles in the second round of primer extension may be selected to optimize the calculation described above. In some embodiments, selecting optimal proportions of first round and second round primer extension may decrease the amount of the output that needs to be removed in the aforementioned calculation step. In some embodiments, 10 primer extension cycles may be performed in the first round and 20 primer extension cycles may be performed in the second round in order to minimize the output data that needs to be removed when calculating the number of nucleic acid sequences belonging to a UMI family. For example, when 10 first round primer extension cycles and 20 second round primer extension cycles are performed, the method may require removing less than the bottom 10% of UMI families by size.

Once a library of immune specific gene nucleic acids have been generated, the library, or a pool of libraries, can be nucleotide sequenced. Any nucleotide sequencing platform can be used as desired, optionally with the universal primer/adapters being selected for the particular nucleotide sequencing platform used. Any of a number of sequencing technologies or sequencing assays can be utilized. The term “Next Generation Sequencing (NGS)” as used herein refers to sequencing methods that allow for massively parallel sequencing of clonally amplified molecules and of single nucleic acid molecules.

Non-limiting examples of sequence assays that are suitable for use with the methods disclosed herein include nanopore sequencing (US Pat. Publ. Nos. 2013/0244340, 2013/0264207, 2014/0134616, 2015/0119259 and 2015/0337366), Sanger sequencing, capillary array sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nature Biotech., 16:381-384 (1998)), sequencing by hybridization (Drmanac et al., Nature Biotech., 16:54-58 (1998), and NGS methods, including but not limited to sequencing by synthesis (e.g., HiSeg™, MiSeg™, or Genome Analyzer, each available from Illumina), sequencing by ligation (e.g., SOLiD™, Life Technologies), ion semiconductor sequencing (e.g., Ion Torrent™, Life Technologies), and SMRT® sequencing (e.g., Pacific Biosciences).

Commercially available sequencing technologies include: sequencing-by-hybridization platforms from Affymetrix Inc. (Sunnyvale, Calif.), sequencing-by-synthesis platforms from Illumina/Solexa (San Diego, Calif) and Helicos Biosciences (Cambridge, Mass.), sequencing-by-ligation platform from Applied Biosystems (Foster City, Calif). Other sequencing technologies include, but are not limited to, the Ion Torrent technology (ThermoFisher Scientific), and nanopore sequencing (Genia Technology from Roche Sequencing Solutions, Santa Clara, Cal.); and Oxford Nanopore Technologies (Oxford, UK).

In some embodiments, the sequencing step involves sequence aligning. In some embodiments, aligning is used to determine a consensus sequence from a plurality of sequences, e.g., a plurality having the same unique molecular ID (UID). The molecular ID is a barcode that can be added to each molecule prior to sequencing or if amplification step is included, prior to the amplification step.

In some embodiments, a consensus sequence is determined from a plurality of sequences all having an identical UID. The sequenced having an identical UID are presumed to derive from the same original molecule through amplification. In other embodiments, UID is used to eliminate artifacts, i.e., variations existing in the progeny of a single molecule (characterized by a particular UID). Such artifacts resulting from PCR errors or sequencing errors can be eliminated.

Once the nucleotide sequences have been determined, one can identify one or more particular immune specific gene sequence, an optionally quantify the number of unique sequence reads from the sample for the one or more particular immune specific gene sequence, thereby determining a relative or absolute quantity of particular immune specific gene sequence in the immune profile of the mother. This can be performed for example on a computer by aligning the sequencing reads to a database of nucleotide sequences, for example a set of known immune specific gene sequences or annotated public databases (e.g., NCBI) and optionally counting the number of unique sequence reads in the alignment.

The particular immune specific gene sequence(s) identified and optionally quantified can include those encoding immune gene products that target alloantigens of an alloimmune disorder or alternatively that provide an immune signature formed by a set of maternal immune gene products that are associated with the development of an alloimmune disorder. In some embodiments, the alloimmune disorder is FNAIT. Exemplary FNAIT-associated alloantigens include, but are not limited to, e.g., HPA-1a. In other embodiments, the alloimmune disorder is HDFN, alloimmune neutropenia, or another alloimmune disorder. In some embodiments, the maternal immune gene products are identified by alignment with previously-identified maternal immune gene products associated with the alloimmune disorder. In other embodiments, protein structure of the maternal immune gene products can be predicted to identify those maternal immune gene products that bind to an alloantigen associated with the alloimmune disorder. In yet other embodiments, a profile of maternal immune gene products, based on their identity and relative quantity, regardless of the presence of their known ability to bind to alloantigens associated with the alloimmune disorder, can be used in a predictive model for development of the alloimmune disorder. For example, in some embodiments, the alloimmune disorder is FNAIT and in some embodiments, the FNAIT-associated alloantigen is HPA-1a, and the maternal immune products are antibodies or T-cell receptors that bind to HPA-1a.

In some embodiments, the quantity of one or more maternal immune gene products is compared to a threshold value, where if the quantity of the maternal immune gene product exceeds the threshold value the development of the alloimmune disorder is predicted. A variety of statistical methods can be used to compare the identities and quantities of maternal immune gene products to threshold values, or alternatively, to results from an earlier sample from the same subject. Exemplary statistical measures can include, for example, a Gini coefficient (see, e.g., Wagner et al., Theory in Biosciences volume 131, pages 281-285 (2012); Muelas, et al. Scientific Reports volume 9, Article number: 17960 (2019) or Joccard index (Prokopenko et al., Bioinformatics, Volume 32, Issue 9, 1 May 2016, Pages 1366-1372; Baharav et al., Patterns Volume 1, Issue 6, 11 Sep. 2020). In some embodiments, a statistical increase (or decrease) of one or more particular maternal immune gene products, either compared to a control or threshold value, or compared to one or more immune profiles from the same woman at an earlier time point (before or during pregnancy) can be used to predict the development of the alloimmune disorder.

A variety of treatments can be administered to the pregnant woman, e.g., following a determination that there is an enhanced likelihood of development of the alloimmune disorder. For example, one or more immunosuppressive agents can be administered to the woman, thereby reducing negative effects of an immune response by the mother against the fetus. Exemplary agents can include, for example immunoglobulins, for example IGIV, corticosteroids, or both. See, e.g., Serrarens-Janssen, et al., Obstet Gynecol Surv. 2008 April; 63(4):239-52. In other embodiments, one or more (e.g., intrauterine) fetal platelet transfusions can be administered. See, e.g., Serrarens-Janssen, et al., Obstet Gynecol Surv. 2008 April; 63(4):239-52. In yet another embodiment, a therapeutic dose of an anti-HPA-1a antibody can be administered. An exemplary antibody is B2GΔnab. See, e.g., Ghevaert C, et al., J Clin Invest. 2008; 118:2929-2938. These and other therapeutics are in development at this time for the prevention and treatment of FNAIT. See, e.g., Geisen C, et al., Poster at International Society of Thrombosis and Haemostasis Conference. 2021 https://rallybio.com/wp-content/uploads/2021/07/RLYB211-ISTH-2021-28jun.2021-vFINAL.pdf. In some embodiments, birth can be induced earlier than would occur naturally, thereby reducing exposure of the fetus to the maternal immune response.

Although the foregoing invention has been described in some detail by way of illustration and example for purposes of clarity of understanding, one of skill in the art will appreciate that many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with the true scope and spirit of the invention being indicated by the following claims.

All publications, patents, patent applications or other documents cited herein are hereby incorporated by reference in their entirety for all purposes to the same extent as if each individual publication, patent, patent application, or other document was individually indicated to be incorporated by reference for all purposes. Where a conflict exists between the instant application and a reference provided herein, the instant application shall dominate.

What is claimed is:

1. A method of quantifying immune specific genes in a pregnant woman carrying a fetus encoding an HPA-1a antigen, wherein the pregnant woman lacks the HPA-1a antigen, the method comprising,
a) providing a reaction mixture comprising:
i) a plurality of structurally different target polynucleotides from the pregnant woman, wherein individual target polynucleotides of the plurality comprise immune cell receptor V, J, and optionally C or D gene regions; and
ii) a plurality of immune cell receptor V gene specific primers, wherein the immune cell receptor V gene specific primers comprise at least 10 structurally distinct primers having the following regions from 5′ to 3′: [5′-Phos], [SPLINT], [BARCODE], and [FW], wherein:
[5′-Phos] comprises a 5′ phosphate;
[SPLINT] comprises an adaptor hybridization site of 2-8 nucleotides in length;
[BARCODE] comprises a barcode region of at least 6 nucleotides in length, wherein each nucleotide of the barcode region is independently selected from the group consisting of N and W; and
[FW] of each immune cell receptor V gene specific primer comprises a structurally distinct region that specifically hybridizes to a framework 1, framework 2, or framework 3 region of an immune cell receptor V gene, wherein the immune cell receptor V gene specific primers are hybridized to the V gene regions of the target polynucleotides;
b) extending the hybridized immune cell receptor V gene specific primers with a polymerase, and then removing un-extended immune cell receptor V gene specific primers, if present, wherein the extended immune cell receptor V gene specific primers comprise at least a portion of the immune cell receptor V region, optionally the immune cell receptor D region, at least a portion of the immune cell receptor C region, and at least a portion of the immune cell receptor J region;
c) hybridizing a first universal adaptor to the [SPLINT] adaptor hybridization site of the extended immune cell receptor V gene specific primers, wherein said universal adaptor is a double-stranded adaptor, comprising: a 5′ single-stranded overhang region which can be hybridized to the SPLINT adaptor hybridization site of said extended primers;
d) ligating the hybridized first universal adapters to the extended immune cell receptor V gene specific primers, and then removing un-ligated adapters, if present;
e) hybridizing a plurality of immune cell receptor J gene specific primers to the J region portions of the extended immune cell receptor V gene specific primers, wherein the immune cell receptor J gene specific primers comprise a 3′ J gene hybridizing region and a 5′ second universal adapter region or hybridizing a plurality of immune cell receptor C gene specific primers to the C region portions of the extended immune cell receptor V gene specific primers, wherein the immune cell receptor C gene specific primers comprise a 3′ C gene hybridizing region and a 5′ second universal adapter region; and
f) extending the hybridized immune cell receptor J gene specific primers or C gene specific primers with a polymerase, thereby forming a plurality of structurally different double-stranded products, each comprising at least a portion of the immune cell receptor V region, optionally the immune cell receptor D region, and at least a portion of the immune cell receptor J region or C region flanked by a first and second universal adapter sequence;
g) amplifying the plurality of structurally different double-stranded products with thereby enriching the plurality of structurally different target polynucleotides comprising immune gene sequences; and
h) determining nucleotide sequences of the plurality of structurally different target polynucleotides comprising the immune specific gene sequences from the products.

2. The method of claim 1, further comprising
i) comparing the quantity or identity of sequences of the plurality of structurally different target polynucleotides comprising the immune gene sequences targeting the HPA-1a antigen from at least one sample to sequences of polynucleotides comprising the immune gene sequences targeting the HPA-1a antigen from a control or from a second sample obtained at an earlier time point in the woman's pregnancy.

3. The method of claim 1, wherein the removing of b) or the removing of d) or both comprise contacting the sample with an exonuclease.

4. The method of claim 1, further comprising:
administering one or more agent or treatment to the woman or fetus to reduce the effect of FNAIT on the fetus.

5. A method of quantifying immune specific genes in a pregnant woman carrying a fetus encoding an HPA-1a antigen, wherein the pregnant woman lacks the HPA-1a antigen, the method comprising,
a) providing a reaction mixture comprising:
i) the plurality of structurally different target polynucleotides from the pregnant woman; and
ii) a plurality of immune cell receptor C gene specific primers, wherein the plurality of structurally distinct primers having the following regions from 5′ to 3′: [5′-Phos], [SPLINT], [BARCODE], and [FW], wherein:
[5′-Phos] comprises a 5′ phosphate;
[SPLINT] comprises an adaptor hybridization site of 2-8 nucleotides in length;
[BARCODE] comprises a barcode region of at least 6 nucleotides in length, wherein each nucleotide of the barcode region is independently selected from the group consisting of N and W; and
[FW] of each immune cell receptor C gene specific primer comprises a structurally distinct region that specifically hybridizes to an immune cell receptor C gene, wherein the immune cell receptor C gene specific primers are hybridized to the C gene regions of the target polynucleotides;
b) extending the hybridized immune cell receptor C gene specific primers with a polymerase, and then removing un-extended immune cell receptor C gene specific primers, if present, wherein the extended immune cell receptor C gene specific primers comprise at least a portion of the immune cell receptor C region, optionally the immune cell receptor D region, at least a portion of the immune cell receptor J region, and at least a portion of the immune cell receptor V region;
c) hybridizing a first universal adaptor to the [SPLINT] adaptor hybridization site of the extended immune cell receptor C gene specific primers;
d) ligating the hybridized first universal adapters to the extended immune cell receptor C gene specific primers, and then removing un-ligated adapters, if present;
e) hybridizing a plurality of immune cell receptor V gene specific primers to the V region portions of the extended immune cell receptor C gene specific primers, wherein the immune cell receptor V gene specific primers comprise a 3′ V gene hybridizing region and a 5′ second universal adapter region; and
f) extending the hybridized immune cell receptor V gene specific primers with a polymerase, thereby forming a plurality of structurally different double-stranded products, each comprising at least a portion of the immune cell receptor V region, optionally the immune cell receptor D region, and at least a portion of the immune cell C region flanked by a first and second universal adapter sequence;
g) amplifying the plurality of second structurally different double-stranded products thereby enriching the plurality of structurally different target polynucleotides comprising an immune gene sequence; and
h) determining nucleotide sequences of the plurality of structurally different target polynucleotides comprising the immune gene sequence from the products.

6. The method of claim 5, further comprising
i) comparing the quantity or identity of sequences of the plurality of structurally different target polynucleotides comprising the immune gene sequences targeting the HPA-1a antigen from at least one sample to sequences of polynucleotides comprising the immune gene sequences targeting the HPA-1a antigen from a control or from a second sample obtained at an earlier time point in the woman's pregnancy.

7. The method of claim 5, wherein the removing of b) or the removing or d) or both comprise contacting the sample with an exonuclease.

8. The method of claim 5, further comprising:
administering one or more agent or treatment to the woman or fetus to reduce the effect of FNAIT on the fetus.

9. A method of quantifying immune specific genes in a pregnant woman carrying a fetus encoding an HPA-1a antigen, wherein the pregnant woman lacks the HPA-1a antigen, the method comprising,
a) providing one or more sample comprising DNA from the pregnant woman;
b) contacting the sample with a plurality of immune cell receptor V gene specific primers, each primer including from 5′ to 3′: [SPLINT1], [BARCODE], and [V], wherein: [SPLINT1] is a first adaptor sequence; [BARCODE] is a unique molecular identifier barcode; and [V] is a sequence capable of hybridizing to an immune cell receptor V gene;
c) hybridizing and extending the V gene specific primers to form a plurality of first double-stranded primer extension products;
d) removing unhybridized V gene specific primers from the first double stranded primer extension products;
e) contacting the sample with a plurality of immune cell receptor J gene specific primers, each primer including from 5′ to 3′: [SPLINT2], and [J], wherein: [SPLINT2] is a second adaptor sequence; and [J] is a sequence capable of hybridizing to an immune cell receptor J gene; and further contacting the sample with a first universal primer capable of hybridizing to the first adaptor sequence;
f) hybridizing and extending the J gene specific primers and the first universal primer to form a plurality of second double-stranded primer extension products;
g) removing unhybridized J gene specific primers and first universal primer from the second double-stranded primer extension products;
h) contacting the sample with first and second universal primers capable of hybridizing to the first and second adaptor sequences;
i) amplifying the plurality of second double-stranded primer extension products thereby enriching the plurality of structurally different target polynucleotides comprising an immune gene sequence; and
j) determining nucleotide sequences of the plurality of structurally different target polynucleotides comprising the immune gene sequence from the products.

10. The method of claim 9, further comprising
k) comparing the quantity or identity of sequences of the plurality of structurally different target polynucleotides comprising the immune gene sequences targeting the HPA-1a antigen from at least one sample to sequences of polynucleotides comprising the immune gene sequences targeting the HPA-1a antigen from a control or from a second sample obtained at an earlier time point in the woman's pregnancy.

11. The method of claim 9, wherein the d) removing or g) removing or both comprise contacting the sample with an exonuclease.

12. The method of claim 9, further comprising:
administering one or more agent or treatment to the woman or fetus to reduce the effect of FNAIT on the fetus.
